(19) United States
(12) Patent Application Publication
(10) Pub. No.: US 2021/0332364 A1
(43) Pub. Date: Oct. 28, 2021

(54) siNA MOLECULES, METHODS OF PRODUCTION AND USES THEREOF

(71) Applicant: PHYZAT BIOPHARMACEUTICALS, LDA, Porto (PT)

(72) Inventor: PATRICIO MANUEL VIEIRA ARAUJO SOARES DA SILVA, PORTO (PT)

(21) Appl. No.: 17/243,173

(22) Filed: Apr. 28, 2021

(30) Foreign Application Priority Data
Apr. 28, 2020 (PT) 116305

(51) Int. Cl. C12N 15/113; A61P 31/14; A61K 45/06

(52) U.S. Cl. CPC C12N 15/1131; A61K 45/06; A61P 31/14

(57) ABSTRACT

The present disclosure relates to method of producing and using short interfering nucleic acids (siNAs) for preventing and treating coronavirus-inflicted infectious conditions. In particular, this disclosure relates to the method of producing and using siNAs for preventing and treating infections by the coronavirus SARS-CoV-2, the causative viral agent of the novel coronavirus disease COVID-19, to mediate gene silencing of viral proteins. The present disclosure is also directed to interfering RNA duplexes and vectors encoding such interfering RNA duplexes.

### CROSS REFERENCE TO RELATED APPLICATION

This patent application claims the benefit and priority of Portugal Patent Application No. 116305 filed on Apr. 28, 2020, the disclosure of which is incorporated by reference herein in its entirety as part of the present application.

### TECHNICAL FIELD

The present disclosure relates to method of producing and using short interfering nucleic acids (siNAs) for preventing and treating coronavirus-inflicted infectious conditions. In particular, this disclosure relates to the method of producing and using siNAs for preventing and treating infections by the coronavirus SARS-CoV-2, the causative viral agent of the novel coronavirus disease COVID-19, to mediate gene silencing of viral proteins. The present disclosure is also directed to interfering RNA duplexes and vectors encoding such interfering RNA duplexes.

### BACKGROUND

Six strains of coronaviruses (CoVs) that are able to infect humans have been identified until 2019. HCoV-OC43, HCoV-229E, HCoV-NL63, and HCoVHKU1 are not highly pathogenic and only cause mild respiratory diseases. SARS-CoV (severe acute respiratory syndrome coronavirus) and MERS-CoV (Middle-East respiratory syndrome coronavirus) have caused two severe epidemics in 2002 and 2012, respectively.

Before efficient antiviral drugs or vaccines were developed for SARS-CoV or MERS-CoV, another outbreak of pneumonia caused by a new coronavirus (SARS-CoV-2) has emerged in Wuhan (China), the virus that causes the disease COVID-19 (Guan et al, 2020; Liu et al., 2020), encompassing asymptomatic infection, mild upper respiratory tract illness, severe viral pneumonia with respiratory failure and even death, and since then spread to multiple continents, leading to WHO's declaration of a Public Health Emergency of International Concern (PHEIC) on 30 Jan. 2020.

No drug or vaccine has yet been approved to treat human coronaviruses. Several options can be envisaged to control or prevent emerging infections by the new coronavirus SARS-CoV-2, including vaccines, monoclonal antibodies, oligonucleotide-based therapies, peptides, interferon therapies and small-molecule drugs (Li & De Clerq, 2020).

SARS-CoV-2 is an enveloped, positive-sense, single-stranded RNA beta-coronavirus. Similar to SARS-CoV or MERS-CoV, the SARS-CoV-2 genome encodes non-structural proteins (such as 3-chymotrypsin-like protease, papain-like protease, helicase, and RNA-dependent RNA polymerase), structural proteins (such as spike glycoprotein) and accessory proteins (Zumla et al., 2016).

The four non-structural proteins mentioned above are key enzymes in the viral life cycle, and the spike (S) glycoprotein is critical for virus-cell receptor interactions during viral entry (Hoffmann et al., 2020).

The spike (S) glycoprotein of coronaviruses facilitates viral entry into target cells. Entry depends on binding of the surface unit, S1, of the S protein to a cellular receptor, which facilitates viral attachment to the surface of target cells. In addition, entry requires S protein priming by cellular proteases, which entails S protein cleavage at the S1/S2 and the S2′ site and allows fusion of viral and cellular membranes, a process driven by the S2 subunit (Hoffmann et al., 2020).

RNA interference (“RNAi”) is a recently discovered mechanism of post-transcriptional gene silencing in which double-stranded RNA corresponding to a gene (or coding region) of interest is introduced into an organism, resulting in degradation of the corresponding mRNA. The phenomenon was originally discovered in Caenorhabditis elegans (Fire et al., 1998).

Unlike antisense technology, the RNAi phenomenon persists for multiple cell divisions before gene expression is regained. The process occurs in at least two steps: an endogenous ribonuclease cleaves the longer dsRNA into shorter, 21-22- or 23-nucleotide-long RNAs, termed “small interfering RNAs” or siRNAs (Hannon, 2002). The siRNA segments then mediate the degradation of the target mRNA. RNAi has been used for gene function determination in a manner similar to but more efficient than antisense oligonucleotides. By making targeted knockouts at the RNA level by RNAi, rather than at the DNA level using conventional gene knockout technology, a vast number of genes can be assayed quickly and efficiently. RNAi is therefore an extremely powerful, simple method for assaying gene function.

RNAi has been shown to be effective in cultured mammalian cells. In most methods described to date, RNAi is carried out by introducing double-stranded RNA into cells by microinjection or by soaking cultured cells in a solution of double-stranded RNA, as well as transfecting the cells with a plasmid carrying a hairpin-structured siRNA expressing cassette under the control of suitable promoters, such as the U6, H1 or cytomegalovirus (“CMV”) promoter (Elbashir et al., 2001; Harborth et al., 2001; Lee et al., 2001; Brummelkamp et al., 2002; Miyagishi et al., 2002; Paddison et al., 2002; Paul et al., 2002; Sui et al., 2002; Xia et al., 2002; Yu et al., 2002). The gene-specific inhibition of gene expression by double-stranded ribonucleic acid is generally described in U.S. Pat. No. 6,506,559, which is incorporated herein by reference. Exemplary use of siRNA technology is further described in Published U.S. Patent Application No. 2003/01090635 and Published U.S. Patent Application No. 20040248174, which are incorporated herein by reference. Davis (Davis, 2009) describes the targeted delivery of siRNA to humans using nanoparticle technology.

Compared with clinically used nonspecific antiviral drugs, a siRNA-spike (S) glycoprotein from SARS-CoV-2 has more advantages for treatment and prevention of SARS-CoV-2 infection. Firstly, the sequence of its target, the spike (S) glycoprotein, is highly conserved. Therefore, a siRNA-spike (S) glycoprotein from SARS-CoV-2 possesses a high genetic barrier to resistance and cannot easily induce drug-resistant mutations. Secondly, a siRNA-spike (S) glycoprotein from SARS-CoV-2 can be used in an intranasal formulation to prevent coronavirus infection. The small containers can be carried easily by persons who will have close contact with infected patients or high-risk populations. Thirdly, a siRNA-spike (S) glycoprotein from SARS-CoV-2 can be used in inhalation formulation for treatment of patients to reduce the viral loads in their lungs, thus attenuating the acute lung injury caused by viral infection and reducing the chance of spreading the virions to the closely contacted persons. The inhalation equipment can be used at home or hotel room, reducing the expense of staying in hospitals. Fourthly, a siRNA-spike (S) glycoprotein from SARS-CoV-2 is expected to be safe to humans because it will be used locally, not systemically, and siRNA drugs are generally safer than chemical drugs.

These facts are disclosed in order to illustrate the technical problem addressed by the present disclosure.

### GENERAL DESCRIPTION

The present disclosure relates to method of producing and using short interfering nucleic acids (siNAs) for preventing and treating coronavirus-inflicted infectious conditions. In particular, it relates to the method of producing and using siNAs for preventing and treating infections by the coronavirus SARS-CoV-2, the causative viral agent of the novel coronavirus disease COVID-19, to mediate gene silencing of viral proteins. The present disclosure is also directed to interfering RNA duplexes and vectors encoding such interfering RNA duplexes.

An object of the present disclosure is to use an RNA interference technique to down regulate the expression of the gene for spike (S) glycoprotein from SARS-CoV-2 in order to treat or prevent the coronavirus SARS-CoV-2 inflicted infectious conditions. The compositions (or molecules) of the disclosure comprises or consists of short interfering nucleic acid molecules (siNA) and related compounds including, but not limited to, siRNA. The present disclosure encompasses compositions and methods of use of siNA including, but not limited to short interfering RNA (siRNA), double-stranded RNA (dsRNA), micro-RNA (miRNA), antagomirs and short hairpin RNA (shRNA) capable of mediating RNA interference. In one embodiment, the siNA molecule of the disclosure can be incorporated into RISC (RNA-induced silencing complex).

A further object of the present disclosure is to provide a siRNA molecule that efficiently down-regulates the expression of the spike (S) glycoprotein from SARS-CoV-2 gene.

Accordingly, in a first aspect, the disclosure relates to a siNA molecule, wherein said molecule specifically targets at least one sequence selected from SEQ ID No 1 to SEQ ID No 339 or a variant thereof. In an alternative embodiment, the disclosure relates to an siNA molecule wherein said molecule specifically targets at least one sequence complementary to at least one sequence selected from SEQ ID No 340 to SEQ ID No 1017 or a variant thereof. In one embodiment, the disclosure relates to an isolated siNA molecule, preferably an isolated siRNA molecule.

In one embodiment, the siNA molecule specifically targets at least one sequence selected from SEQ ID No 35, SEQ ID No 36, SEQ ID No 113, SEQ ID No 114, SEQ ID No 161, SEQ ID No 162, SEQ ID No 181, SEQ ID No 217, SEQ ID No 223, SEQ ID No 224, SEQ ID No 226, SEQ ID No 227, SEQ ID No 230, SEQ ID No 231, SEQ ID No 303, SEQ ID No 304, SEQ ID No 305, SEQ ID No 307, SEQ ID No 308, SEQ ID No 309, SEQ ID No 327, SEQ ID No 328, SEQ ID No 329, SEQ ID No 332, SEQ ID No 333, SEQ ID No 337, SEQ ID No 338, or a variant thereof. Preferably, the siNA molecule targets a sequence selected from SEQ ID No 36, SEQ ID No 113, SEQ ID No 161, SEQ ID No 162, SEQ ID No 181, SEQ ID No 217, SEQ ID No 224, SEQ ID No 227, SEQ ID No 309, SEQ ID No 327, SEQ ID No 329, SEQ ID No 332, SEQ ID No 333, or a variant thereof. Preferably, the siNA molecule reduces expression of the spike (S) glycoprotein from SARS-CoV-2 gene when expressed into a cell.

In a further embodiment, the siNA preferably comprises a double-stranded RNA molecule, whose antisense strand is substantially complementary to any of SEQ ID No 1 to SEQ ID No 339, more preferably SEQ ID No 35, SEQ ID No 36, SEQ ID No 113, SEQ ID No 114, SEQ ID No 161, SEQ ID No 162, SEQ ID No 181, SEQ ID No 217, SEQ ID No 223, SEQ ID No 224, SEQ ID No 226, SEQ ID No 227, SEQ ID No 230, SEQ ID No 231, SEQ ID No 303, SEQ ID No 304, SEQ ID No 305, SEQ ID No 307, SEQ ID No 308, SEQ ID No 309, SEQ ID No 327, SEQ ID No 328, SEQ ID No 329, SEQ ID No 332, SEQ ID No 333, SEQ ID No 337, SEQ ID No 338, or a variant thereof, even more preferably SEQ ID No 36, SEQ ID No 113, SEQ ID No 161, SEQ ID No 162, SEQ ID No 181, SEQ ID No 217, SEQ ID No 224, SEQ ID No 227, SEQ ID No 309, SEQ ID No 327, SEQ ID No 329, SEQ ID No 332, SEQ ID No 333, or a variant thereof, and its sense strand will comprise an RNA sequence complementary to the antisense strand, wherein both strands are hybridised by standard base pairing between nucleotides.

In a further embodiment, said sense strand comprises or consists of a sequence selected from SEQ ID No 340 to SEQ ID No 678, preferably SEQ ID No 374, SEQ ID No 375, SEQ ID No 452, SEQ ID No 453, SEQ ID No 500, SEQ ID No 501, SEQ ID No 520, SEQ ID No 556, SEQ ID No 562, SEQ ID No 563, SEQ ID No 565, SEQ ID No 566, SEQ ID No 569, SEQ ID No 570, SEQ ID No 642, SEQ ID No 643, SEQ ID No 644, SEQ ID No 646, SEQ ID No 647, SEQ ID No 648, SEQ ID No 666, SEQ ID No 667, SEQ ID No 668, SEQ ID No 671, SEQ ID No 672, SEQ ID No 676, SEQ ID No 677, or a variant thereof; more preferably SEQ ID No 375, SEQ ID No 452, SEQ ID No 500, SEQ ID No 501, SEQ ID No 520, SEQ ID No 556, SEQ ID No 563, SEQ ID No 566, SEQ ID No 648, SEQ ID No 666, SEQ ID No 668, SEQ ID No 671 or SEQ ID No 672 or a variant thereof.

In a further embodiment, said antisense strand comprises or consists of a sequence selected from SEQ ID No 679 to SEQ ID No 1017, preferably SEQ ID No 713, SEQ ID No 714, SEQ ID No 791, SEQ ID No 792, SEQ ID No 839, SEQ ID No 840, SEQ ID No 859, SEQ ID No 895, SEQ ID No 901, SEQ ID No 902, SEQ ID No 904, SEQ ID No 905, SEQ ID No 908, SEQ ID No 909, SEQ ID No 981, SEQ ID No 982, SEQ ID No 983, SEQ ID No 985, SEQ ID No 986, SEQ ID No 987, SEQ ID No 1005, SEQ ID No 1006, SEQ ID No 1007, SEQ ID No 1010, SEQ ID No 1011, SEQ ID No 1015, SEQ ID No 1016, or a variant thereof. More preferably, SEQ ID No 714, SEQ ID No 791, SEQ ID No 839, SEQ ID No 840, SEQ ID No 859, SEQ ID No 895, SEQ ID No 902, SEQ ID No 905, SEQ ID No 987, SEQ ID No 1005, SEQ ID No 1007, SEQ ID No 1010, SEQ ID No 1011, or a variant thereof.

Within the meaning of the present disclosure “substantially complementary” to a target mRNA sequence, may also be understood as “substantially identical” to said target sequence. “Identity” as is known by one of ordinary skill in the art, is the degree of sequence relatedness between nucleotide sequences as determined by matching the order and identity of nucleotides between sequences. In one embodiment the antisense strand of an siRNA having 80%, and between 80% up to 100% complementarity, for example, 85%, 86%, 87%, 88%, 89%, 90%, 91%, 92%, 93%, 94%, 95%, 96%, 97% or 99% complementarity, to the target mRNA sequence are considered substantially complementary and may be used in the present disclosure. The percentage of complementarity describes the percentage of contiguous nucleotides in a first nucleic acid molecule that can base pair in the Watson-Crick sense with a set of contiguous nucleotides in a second nucleic acid molecule.

A gene is “targeted” by a siNA according to the present disclosure when, for example, the siNA molecule selectively decreases or inhibits the expression of the gene. The phrase “selectively decrease or inhibit” as used herein encompasses siNAs that affect expression of the spike (S) glycoprotein from SARS-CoV-2. Alternatively, a siNA targets a gene when the siNA hybridizes under stringent conditions to the gene transcript, i.e. its mRNA. Capable of hybridizing “under stringent conditions” means annealing to the target mRNA region, under standard conditions, e.g., high temperature and/or low salt content which tend to disfavor hybridization. A suitable protocol (involving 0.1×SSC, 68° C. for 2 hours) is described in Maniatis, T., et al., Molecular Cloning: A Laboratory Manual, Cold Spring Harbor Laboratory, 1982, at pages 387-389.

Nucleic acid sequences cited herein are written in a 5′ to 3′ direction unless indicated otherwise. The term “nucleic acid” refers to either DNA or RNA or a modified form thereof comprising the purine or pyrimidine bases present in DNA (adenine “A”, cytosine “C”, guanine “G”, thymine “T”) or in RNA (adenine “A”, cytosine “C”, guanine “G”, uracil “U”). Interfering RNAs provided herein may comprise “T” bases, for example at 3′ ends, even though “T” bases do not naturally occur in RNA. In some cases, these bases may appear as “dT” to differentiate deoxyribonucleotides present in a chain of ribonucleotides.

In one embodiment of the disclosure, the siNA molecule is 40 base pairs or fewer in length. Preferably, the siNA molecule is 19 to 25 base pairs in length. In one embodiment, the siNA comprises or consists of 21 nucleotides double-stranded region. In one embodiment, the siNA comprises or consists of a 22 nucleotides double-stranded region. Preferably, the siNA has a sense and an anti-sense strand. In an alternative embodiment, the siNA molecule comprises or consists of 19 nucleotides double-stranded region. In one embodiment, the siNA has blunt ends. In an alternative embodiment, the siNA has 5′ and/or 3′ overhangs. Preferably the overhangs are between 1 to 5 nucleotides, more preferably, 2 nucleotide overhangs. The overhangs may be ribonucleic acids, or deoxyribonucleic acids.

In one embodiment, the siNA molecule according to the disclosure comprises a chemical modification. Preferably, the chemical modification is on the sense strand, the antisense strand or both. Phosphorothioate (PS)- or boranophosphate (BS)-modified siRNAs have substantial nuclease resistance. Silencing by siRNA duplexes is also compatible with some types of 2′-sugar modifications: 2′-H, 2′-O-methyl, 2′-O-methoxyethyl, 2′-fluoro (2′-F), locked nucleic acid (LNA) and ethylene-bridge nucleic acid (ENA).

In one embodiment, the 5′ or 3′ overhangs are dinucleotides, preferably thymidine dinucleotide. In an embodiment, the 5′ or 3′ overhangs are deoxythymidines. In one embodiment, the sense strand comprises at least one, preferably two 3′ overhangs. Preferably, said sense strand comprises at least one, preferably two 3′ deoxythymidines. In an alternative embodiment, the antisense strand comprises at least one, preferably two 3′ overhangs. Preferably, said sense strand comprises at least one, preferably two 3′ deoxythymidines. In a further preferred embodiment, both the sense and antisense strands comprise 3′ overhangs as described herein.

By “variant” as used herein is meant a sequence with 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic or ribonucleic acid sequence.

By “down-regulating” is meant a decrease in the expression of spike (S) glycoprotein from SARS-CoV-2 mRNA by up to or more than 10%, 15% 20%, 25%, 30%, 35%, 40%, 45% 50%, 55% 60%, 65%, 70%, 75%, 80%, 85%, 90%, 95% when compared to the level in a control. Alternatively, the siNA molecule described herein may abolish SARS-CoV-2 spike (S) glycoprotein expression. The term “abolish” means that no expression of SARS-CoV-2 spike (S) glycoprotein is detectable or that no functional SARS-CoV-2 spike (S) glycoprotein is produced. For example, a reduction in the expression and/or protein levels of at least SARS-CoV-2 spike (S) glycoprotein expression may be a measure of protein and/or nucleic acid levels and can be measured by any technique known to the skilled person, such as, but not limited to, any form of gel electrophoresis or chromatography (e.g. HPLC).

Notably, in some embodiments, the siNA molecule (either the 5′ or 3′ strand or both) may begin with at least one, preferably two alanine nucleotides. Alternatively, if the target sequence starts with one or two alanine sequences, these may not be included (targeted) in the siNA molecule.

In one embodiment, the target sequence may be characterised by at least one, preferably two alanine nucleotides at the 3′ end of the sequence, and/or the target sequence lacks at least one, preferably two alanine nucleotides at the 5′ end of the sequence, and/or the target sequence lacks two consecutive alanine nucleotides within the sequence. In a preferred embodiment, the siNA molecules of the disclosure are characterised in that they target sequences with the above properties.

In one embodiment a plurality of species of siNA molecule are used, wherein said plurality of siNA molecules are targeted to the same or a different mRNA species.

In one embodiment, the siNA is selected from dsRNA, siRNA or shRNA. Preferably, the siNA is siRNA.

In one embodiment, an isolated or synthetic siNA molecule comprising at least a sequence 88% identical to SEQ ID No 340 to SEQ ID No 678, preferably SEQ ID No 374, SEQ ID No 375, SEQ ID No 452, SEQ ID No 453, SEQ ID No 500, SEQ ID No 501, SEQ ID No 520, SEQ ID No 556, SEQ ID No 562, SEQ ID No 563, SEQ ID No 565, SEQ ID No 566, SEQ ID No 569, SEQ ID No 570, SEQ ID No 642, SEQ ID No 643, SEQ ID No 644, SEQ ID No 646, SEQ ID No 647, SEQ ID No 648, SEQ ID No 666 to SEQ ID No 668, SEQ ID No 672, SEQ ID No 676 and SEQ ID No 677, more preferably SEQ ID No 375, SEQ ID No 452, SEQ ID No 500, SEQ ID No 501, SEQ ID No 520, SEQ ID No 556, SEQ ID No 563, SEQ ID No 566, SEQ ID No 648, SEQ ID No 666, SEQ ID No 668, SEQ ID No 671, and SEQ ID No 672. Preferably at least 89% identical, or at least 90% identical, or at least 91% identical, or at least 92% identical, or at least 93% identical, or at least 94% identical, or at least 95% identical, or at least 96% identical, or at least 97% identical, or at least 98% identical, or at least 99% identical, or 100% identical to SEQ ID No 374, SEQ ID No 375, SEQ ID No 452, SEQ ID No 453, SEQ ID No 500, SEQ ID No 501, SEQ ID No 520, SEQ ID No 556, SEQ ID No 562, SEQ ID No 563, SEQ ID No 565, SEQ ID No 566, SEQ ID No 569, SEQ ID No 570, SEQ ID No 642, SEQ ID No 643, SEQ ID No 644, SEQ ID No 646, SEQ ID No 647, SEQ ID No 648, SEQ ID No 666, SEQ ID No 667, SEQ ID No 668, SEQ ID No 671, SEQ ID No 672, SEQ ID No 676, SEQ ID No 677, more preferably SEQ ID No 375, SEQ ID No 452, SEQ ID No 500, SEQ ID No 501, SEQ ID No 520, SEQ ID No 556, SEQ ID No 563, SEQ ID No 566, SEQ ID No 648, SEQ ID No 666, SEQ ID No 668, SEQ ID No 671, and SEQ ID No 672.

In one embodiment, an isolated or synthetic siNA molecule comprising at least a sequence 88% identical SEQ ID No 713, SEQ ID No 714, SEQ ID No 791, SEQ ID No 792, SEQ ID No 839, SEQ ID No 840, SEQ ID No 859, SEQ ID No 895, SEQ ID No 901, SEQ ID No 902, SEQ ID No 904, SEQ ID No 905, SEQ ID No 908, SEQ ID No 909, SEQ ID No 981, SEQ ID No 982, SEQ ID No 983, SEQ ID No 985, SEQ ID No 986, SEQ ID No 987, SEQ ID No 1005, SEQ ID No 1006, SEQ ID No 1007, SEQ ID No 1010, SEQ ID No 1011, SEQ ID No 1015 or SEQ ID No 1016; more preferably SEQ ID No SEQ ID No 714, SEQ ID No 791, SEQ ID No 839, SEQ ID No 840, SEQ ID No 859, SEQ ID No 895, SEQ ID No 902, SEQ ID No 905, SEQ ID No 987, SEQ ID No 1005, SEQ ID No 1007, SEQ ID No 1010 and SEQ ID No 1011. Preferably at least 89% identical, or at least 90% identical, or at least 91% identical, or at least 92% identical, or at least 93% identical, or at least 94% identical, or at least 95% identical, or at least 96% identical, or at least 97% identical, or at least 98% identical, or at least 99% identical, or 100% identical to SEQ ID No 713, SEQ ID No 714, SEQ ID No 791, SEQ ID No 792, SEQ ID No 839, SEQ ID No 840, SEQ ID No 859, SEQ ID No 895, SEQ ID No 901, SEQ ID No 902, SEQ ID No 904, SEQ ID No 905, SEQ ID No 908, SEQ ID No 909, SEQ ID No 981, SEQ ID No 982, SEQ ID No 983, SEQ ID No 985, SEQ ID No 986, SEQ ID No 987, SEQ ID No 1005, SEQ ID No 1006, SEQ ID No 1007, SEQ ID No 1010, SEQ ID No 1011, SEQ ID No 1015 or SEQ ID No 1016; more preferably SEQ ID No SEQ ID No 714, SEQ ID No 791, SEQ ID No 839, SEQ ID No 840, SEQ ID No 859, SEQ ID No 895, SEQ ID No 902, SEQ ID No 905, SEQ ID No 987, SEQ ID No 1005, SEQ ID No 1007, SEQ ID No 1010 and SEQ ID No 1011.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (over the whole the sequence) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. The sequence identity values, which are indicated in the present subject matter as a percentage were determined over the entire amino acid sequence, using BLAST with the default parameters.

In a further embodiment, the disclosure relates to a siNA molecule, as herein described for use as a medicament. In one embodiment, the disclosure relates to a siNA for use in the treatment of a disorder characterised by increased expression levels (compared to the levels in a healthy subject) of SARS-CoV-2 spike (S) protein.

In another aspect of the disclosure, there is provided a siNA molecule, as described herein for preventing and treating infections by the coronavirus SARS-CoV-2.

In a further aspect, the disclosure relates to the use of at least one siNA molecule, as described herein in the preparation of a medicament for preventing and treating infections by the coronavirus SARS-CoV-2.

In another aspect, the disclosure relates to a method for preventing and treating infections by the coronavirus SARS-CoV-2, the method comprising administering at least one siNA molecule, as described herein, to a patient or subject in need thereof.

In one embodiment, infection by the coronavirus SARS-CoV-2 is selected from asymptomatic infection, mild upper respiratory tract illness, severe viral pneumonia and with respiratory failure.

In another aspect of the disclosure there is provided a pharmaceutical composition comprising at least one siNA molecule as described herein and a pharmaceutically acceptable carrier.

In a further aspect of the disclosure there is provided a method, preferably an in vitro method of inhibiting spike (S) glycoprotein expression for virus-cell receptor interactions during viral entry into a cell, the method comprising administering a siNA as defined herein to a cell. Preferably, the viral entry is promoted by the spike (S) glycoprotein. In one embodiment, spike (S) glycoprotein expression in a cell is inhibited by up to or more than 10%, 20%, 30%, 40%, 50%, 60%, 70%, 80% or 90% when compared to the level in a control.

In a further aspect of the disclosure there is provided a method, preferably an in vitro method of inhibiting spike (S) glycoprotein for virus-cell receptor interactions during viral entry into a cell, the method comprising administering a siNA as defined herein to a cell. Preferably, the viral entry is promoted by the spike (S) glycoprotein. In one embodiment, viral entry into a cell is inhibited by up to or more than 10%, 20%, 30%, 40%, 50%, 60%, 70%, 80% or 90% when compared to the level in a control

In a yet further aspect of the disclosure, there is provided a method of reducing viral infection, preferably in a patient, the method comprising administering at least one siNA as described herein. In one embodiment, said decrease in viral infection may be up to or more than 10%, 20%, 30%, 40%, 50%, 60%, 70%, 80% or 90% when compared to the level in a control.

In another embodiment, the disclosure relates to methods of reducing viral entry into a cell comprising treating the cells with an siNA of the disclosure in combination with one or more anti-viral agents known in the art, preferably wherein the anti-viral agent comprises a nucleoside analogue antiviral agent and most preferably favipiravir, ribavirin, remdesivir and galidesivir.

The disclosure also relates to methods of treating viral infection comprising administrating an siNA of the disclosure in combination with one or more anti-viral agents known in the art, preferably to a patient in need thereof, preferably wherein the anti-viral agent comprises an anti-nucleoside agent, more preferably an antiviral agent and most preferably favipiravir, ribavirin, remdesivir and galidesivir. The disclosure further relates to pharmaceutical compositions comprising the siNA of the disclosure and the one or more anti-viral agent.

In another embodiment the disclosure relates to methods for increasing the efficacy of an anti-viral therapy given to a patient comprising administering an siNA of the disclosure in combination with the therapy. Said increase in efficacy may be up to or more than 10%, 20%, 30%, 40%, 50%, 60%, 70%, 80% or 90% when compared to the efficacy of either administration of siNA or the anti-viral agent alone.

The disclosure also relates to methods of treating viral infection comprising administrating an siNA of the disclosure in combination with one or more transmembrane protease serine 2 (TMPRSS2) inhibitors known in the art, preferably to a patient in need thereof, preferably wherein the anti-TMPRSS2 agent comprises an, more preferably an anti-TMPRSS2 agent and most preferably camostat or nafamostat. The disclosure further relates to pharmaceutical compositions comprising the siNA of the disclosure and the one or more anti-TMPRSS2 agent.

In another embodiment the disclosure relates to methods for increasing the efficacy of TMPRSS2 inhibition therapy given to a patient comprising administering an siNA of the disclosure in combination with the therapy. Said increase in efficacy may be up to or more than 10%, 20%, 30%, 40%, 50%, 60%, 70%, 80% or 90% when compared to the efficacy of either administration of siNA or the TMPRSS2 inhibition therapy alone.

### DETAILED DESCRIPTION

The present disclosure relates to method of producing and using siNAs for preventing and treating coronavirus-inflicted infectious conditions. siNAs for preventing and treating infections by the coronavirus SARS-CoV-2, the causative viral agent of the novel coronavirus disease COVID-19, to mediate gene silencing of viral proteins.

According to a second aspect of the present disclosure, there is provided a method of treating or preventing by the coronavirus SARS-CoV-2, the causative viral agent of the novel coronavirus disease COVID-19, comprising administering to an individual an effective amount of a siRNA that inhibits spike (S) glycoprotein gene expression, wherein the siRNA comprises a sense spike (S) glycoprotein nucleic acid and an antisense spike (S) glycoprotein nucleic acid. The present disclosure also provides a method of treating or preventing coronavirus-inflicted infectious conditions comprising administering to an individual an effective amount of a vector encoding the siRNA that inhibits spike (S) glycoprotein gene expression.

The spike (S) glycoprotein of coronaviruses, namely the SARS-CoV-2 spike (S) glycoprotein, facilitates viral entry into target cells. Entry depends on binding of the surface unit, S1, of the S protein to a cellular receptor, which facilitates viral attachment to the surface of target cells. In addition, entry requires S protein priming by cellular proteases, which entails S protein cleavage at the S1/S2 and the S2′ site and allows fusion of viral and cellular membranes, a process driven by the S2 subunit. The present disclosure is based on the surprising discovery that small interfering RNAs (siRNAs) selective for SARS-CoV-2 spike (S) glycoprotein are effective preventing and treating the coronavirus SARS-CoV-2 inflicted infectious conditions. In particular, infections by the coronavirus SARS-CoV-2 selected from asymptomatic infection, mild upper respiratory tract illness, severe viral pneumonia and with respiratory failure.

The siRNA or vector encoding the siRNA, or the medicament comprising the siRNA or vector encoding the siRNA, may be administered to an individual by topical application, nasal application, inhalation administration, subcutaneous injection or deposition, subcutaneous infusion, intravenous injection, intravenous infusion.

According to a third aspect of the present disclosure there is provided an in vitro method of inhibiting the expression of the spike (S) glycoprotein gene in a cell comprising contacting the cell with siNA that inhibits spike (S) glycoprotein gene expression as described herein. In one embodiment, said siRNA comprises a sense spike (S) glycoprotein nucleic acid and an anti-sense spike (S) glycoprotein nucleic acid, wherein the sense spike (S) glycoprotein nucleic acid is substantially identical to a target sequence contained within spike (S) glycoprotein mRNA and the anti-sense spike (S) glycoprotein nucleic acid is complementary to the sense spike (S) glycoprotein nucleic acid. The present disclosure also provides an in vitro method of inhibiting the expression of the spike (S) glycoprotein gene in a cell comprising contacting the cell with a vector encoding a siRNA that inhibits spike (S) glycoprotein gene expression, said siRNA comprises a sense spike (S) glycoprotein nucleic acid and an anti-sense spike (S) glycoprotein nucleic acid, wherein the sense spike (S) glycoprotein nucleic acid is substantially identical to a target sequence contained within spike (S) glycoprotein mRNA and the anti-sense spike (S) glycoprotein nucleic acid is complementary to the sense spike (S) glycoprotein nucleic acid.

Expression of the gene may be inhibited by introduction of a double stranded ribonucleic acid (dsRNA) molecule into the cell in an amount sufficient to inhibit expression of the spike (S) glycoprotein gene.

The siRNAs used in the disclosure are believed to cause the RNAi-mediated degradation of spike (S) glycoprotein from SARS-CoV-2 mRNA so that the protein product of the spike (S) glycoprotein from SARS-CoV-2 gene is not produced or is produced in reduced amounts. The siRNAs used in the disclosure can be used to alter gene expression in a cell in which expression of spike (S) glycoprotein from SARS-CoV-2 is initiated, e.g., as a result of SARS-CoV-2-inflicted infectious conditions such as in asymptomatic infection, mild upper respiratory tract illness, severe viral pneumonia and with respiratory failure. Binding of the siRNA to a spike (S) glycoprotein mRNA transcript in a cell results in a reduction in spike (S) glycoprotein production by the infected cell.

The term “siRNA” is used to mean a double stranded RNA molecule which prevents translation of a target mRNA. Standard techniques of introducing siRNA into the cell are used, including those in which DNA is a template from which RNA is transcribed. The siRNA that inhibits spike (S) glycoprotein from SARS-CoV-2 gene expression includes a sense spike (S) glycoprotein from SARS-CoV-2 nucleic acid sequence and an antisense spike (S) glycoprotein from SARS-CoV-2 nucleic acid sequence. The siRNA may be constructed such that a single transcript has both the sense and complementary antisense sequences from the target gene, e.g., in the form of a hairpin.

The siRNA preferably comprises short double-stranded RNA that is targeted to the target mRNA, i.e., spike (S) glycoprotein from SARS-CoV-2 mRNA. The siRNA comprises a sense RNA strand and a complementary antisense RNA strand annealed together by standard Watson-Crick base-pairing interactions (hereinafter “base-paired”). The sense strand comprises a nucleic acid sequence which is substantially identical to a target sequence contained within the spike (S) glycoprotein from SARS-CoV-2 mRNA.

The terms “sense/antisense sequences” and “sense/antisense strands” are used interchangeable herein to refer to the parts of the siRNA of the present disclosure that are substantially identical (sense) to the target SARS-CoV-2 mRNA sequence or substantially complementary (antisense) to the target spike (S) glycoprotein from SARS-CoV-2 mRNA sequence.

As used herein, a nucleic acid sequence “substantially identical” to a target sequence contained within the target mRNA is a nucleic acid sequence which is identical to the target sequence, or which differs from the target sequence by one or more nucleotides. Preferably, the substantially identical sequence is identical to the target sequence or differs from the target sequence by one, two or three nucleotides, more preferably by one or two nucleotides and most preferably by only 1 nucleotide. Sense strands which comprise nucleic acid sequences substantially identical to a target sequence are characterized in that siRNA comprising such a sense strand induces RNAi-mediated degradation of mRNA containing the target sequence. For example, an siRNA of the disclosure can comprise a sense strand comprising a nucleic acid sequence which differs from a target sequence by one, two, three or more nucleotides, as long as RNAi-mediated degradation of the target mRNA is induced by the siRNA.

The sense and antisense strands of the siRNA can comprise two complementary, single-stranded RNA molecules or can comprise a single molecule in which two complementary portions are base-paired and are covalently linked by a single-stranded “hairpin” area. That is, the sense region and antisense region can be covalently connected via a linker molecule. The linker molecule can be a polynucleotide or non-nucleotide linker. The siRNA can also contain alterations, substitutions or modifications of one or more ribonucleotide bases. For example, the present siRNA can be altered, substituted or modified to contain one or more, preferably 0, 1, 2 or 3, deoxyribonucleotide bases. Preferably, the siRNA does not contain any deoxyribonucleotide bases.

The siRNA can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA; modifications that make the siRNA resistant to nuclease digestion (e.g., the use of 2′-substituted ribonucleotides or modifications to the sugar-phosphate backbone); or the substitution of one or more, preferably 0, 1, 2 or 3, nucleotides in the siRNA with deoxyribonucleotides.

Degradation can be delayed or avoided by a wide variety of chemical modifications that include alterations in the nucleobases, sugars and the phosphate ester backbone of the siRNAs. All of these chemically modified siRNAs are still able to induce siRNA-mediated gene silencing provided that the modifications were absent in specific regions of the siRNA and included to a limited extent. In general, backbone modifications cause a small loss in binding affinity, but offer nuclease resistance. Phosphorothioate (PS)- or boranophosphate (BS)-modified siRNAs have substantial nuclease resistance. Silencing by siRNA duplexes is also compatible with some types of 2′-sugar modifications: 2′-H, 2′-O-methyl, 2′-O-methoxyethyl, 2′-fluoro (2′-F), locked nucleic acid (LNA) and ethylene-bridge nucleic acid (ENA). Suitable chemical modifications are well known to those skilled in the art.

The siRNA used in the present disclosure is a double-stranded molecule comprising a sense strand and an antisense strand, wherein the sense strand comprises or consists of a ribonucleotide sequence corresponding to spike (S) glycoprotein from SARS-CoV-2 target sequence, and wherein the antisense strand comprises a ribonucleotide sequence which is complementary to said sense strand, wherein said sense strand and said antisense strand hybridize to each other to form said double-stranded molecule, and wherein said double-stranded molecule, when introduced into a cell expressing the spike (S) glycoprotein from SARS-CoV-2 gene, inhibits expression of the said gene. As indicated further below, said spike (S) glycoprotein from SARS-CoV-2 target sequence preferably comprises at least about 15 contiguous, more preferably 19 to 25, and most preferably about 19 to 21 contiguous nucleotides selected from the group consisting of from SEQ ID No 35, SEQ ID No 36, SEQ ID No 113, SEQ ID No 114, SEQ ID No 161, SEQ ID No 162, SEQ ID No 181, SEQ ID No 217, SEQ ID No 223, SEQ ID No 224, SEQ ID No 226, SEQ ID No 227, SEQ ID No 230, SEQ ID No 231, SEQ ID No 303, SEQ ID No 304, SEQ ID No 305, SEQ ID No 307, SEQ ID No 308, SEQ ID No 309, SEQ ID No 327, SEQ ID No 328, SEQ ID No 329, SEQ ID No 332, SEQ ID No 333, SEQ ID No 337, SEQ ID No 338, or variants thereof.

The siRNA used in the present disclosure can be obtained using a number of techniques known to those of skill in the art. For example, the siRNA can be chemically synthesized or recombinantly produced using methods known in the art, such as the Drosophila in vitro system described in U.S. published application 2002/0086356, the entire disclosure of which is herein incorporated by reference. The siRNA may be chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Biospring (Frankfurt, Germany), ChemGenes (Ashland, Mass., USA), Dharmacon Research (Lafayette, Colo., USA), Glen Research (Sterling, Va., USA), Proligo (Hamburg, Germany), Sigma-Aldrich (St. Louis, Mo. USA) and Thermo Fisher Scientific (Waltham, Mass. USA).

The siRNA can also be expressed from recombinant circular or linear DNA vectors using any suitable promoter. Suitable promoters for expressing siRNA from a vector include, for example, the U6 or H1 RNA pol III promoter sequences and the cytomegalovirus promoter. Selection of other suitable promoters is within the skill in the art. The vector can also comprise inducible or regulable promoters for expression of the siRNA in a particular tissue or in a particular intracellular environment.

The siRNA expressed from a vector can either be isolated from cultured cell expression systems by standard techniques, or can be expressed intracellularly. The vector can be used to deliver the siRNA to cells in vivo, e.g., by intracellularly expressing the siRNA in vivo. siRNA can be expressed from a vector either as two separate, complementary RNA molecules, or as a single RNA molecule with two complementary regions. Selection of vectors suitable for expressing the siRNA, methods for inserting nucleic acid sequences for expressing the siRNA into the vector, and methods of delivering the vector to the cells of interest are well known to those skilled in the art.

The siRNA can also be expressed from a vector intracellularly in vivo. As used herein, the term “vector” means any nucleic acid- and/or viral-based technique used to deliver a desired nucleic acid. Any vector capable of accepting the coding sequences for the siRNA molecule(s) to be expressed can be used, including plasmids, cosmids, naked DNA, optionally condensed with a condensing agent, and viral vectors. Suitable viral vectors include vectors derived from adenovirus (AV); adeno-associated virus (AAV); retroviruses (e.g., lentiviruses (LV), Rhabdoviruses, murine leukemia virus); herpes virus, and the like. The tropism of viral vectors can be modified by pseudotyping the vectors with envelope proteins or other surface antigens from other viruses, or by substituting different viral capsid proteins, as appropriate. When the vector is a lentiviral vector it is preferably pseudotyped with surface proteins from vesicular stomatitis virus, rabies virus, Ebola virus or Mokola virus.

Vectors are produced for example by cloning the spike (S) glycoprotein from SARS-CoV-2 target sequence into an expression vector so that operatively-linked regulatory sequences flank the spike (S) glycoprotein sequence in a manner that allows for expression (by transcription of the DNA molecule) of both strands (Lee et al., 2002). An RNA molecule that is antisense to spike (S) glycoprotein mRNA is transcribed by a first promoter (e.g., a promoter sequence 3′ of the cloned DNA) and an RNA molecule that is the sense strand for the spike (S) glycoprotein mRNA is transcribed by a second promoter (e. g., a promoter sequence 5′ of the cloned DNA). The sense and antisense strands hybridize in vivo to generate siRNA constructs for silencing of the spike (S) glycoprotein gene. Alternatively, two vectors are utilized to create the sense and anti-sense strands of a siRNA construct. Cloned spike (S) glycoprotein can encode a construct having secondary structure, e. g., hairpins, wherein a single transcript has both the sense and complementary antisense sequences from the target gene. Such a transcript encoding a construct having secondary structure, will preferably comprises a single-stranded ribonucleotide sequence (loop sequence) linking said sense strand and said antisense strand.

The siRNA is preferably isolated. As used herein, “isolated” means synthetic, or altered or removed from the natural state through human intervention. For example, a siRNA naturally present in a living animal is not “isolated,” but a synthetic siRNA, or a siRNA partially or completely separated from the coexisting materials of its natural state is “isolated.” An isolated siRNA can exist in substantially purified form, or can exist in a non-native environment such as, for example, a cell into which the siRNA has been delivered. By way of example, siRNA which are produced inside a cell by natural processes, but which are produced from an “isolated” precursor molecule, are themselves “isolated” molecules. Thus, an isolated dsRNA can be introduced into a target cell, where it is processed by the Dicer protein (or its equivalent) into isolated siRNA.

As used herein, “inhibit” means that the activity of the spike (S) glycoprotein gene expression product or level of the spike (S) glycoprotein gene expression product is reduced below that observed in the absence of the siRNA molecule of the disclosure. The inhibition with a siRNA molecule preferably is significantly below that level observed in the presence of an inactive or attenuated molecule that is unable to mediate an RNAi response. Inhibition of gene expression with the siRNA molecule is preferably significantly greater in the presence of the siRNA molecule than in its absence. Preferably, the siRNA inhibits the level of spike (S) glycoprotein gene expression by at least 10%, more preferably at least 50% and most preferably at least 75%.

Preferably the siRNA molecule inhibits spike (S) glycoprotein gene expression so that the protein product of the spike (S) glycoprotein from SARS-CoV-2 gene is not produced or is produced in reduced amounts. By inhibiting spike (S) glycoprotein expression for virus-cell receptor interactions during viral entry into a cell is meant that the treated cell produces at a lower rate or has decreased the viral protein that allows viral entry than an untreated cell. The spike (S) glycoprotein from SARS-CoV-2 is measured by mRNA or protein assays known in the art.

As used herein, an “isolated nucleic acid” is a nucleic acid removed from its original environment (e. g., the natural environment if naturally occurring) and thus, synthetically altered from its natural state. In the present disclosure, isolated nucleic acid includes DNA, RNA, and derivatives thereof. When the isolated nucleic acid is RNA or derivatives thereof, base “t” should be replaced with “u” in the nucleotide sequences.

As used herein, the term “complementary” refers to Watson-Crick or Hoogsteen base pairing between nucleotides units of a polynucleotide, and the term “binding” means the physical or chemical interaction between two polypeptides or compounds or associated polypeptides or compounds or combinations thereof.

As used herein, the phrase “highly conserved sequence region” means a nucleotide sequence of one or more regions in a target gene does not vary significantly from one generation to the other or from one biological system to the other.

As used herein, the term “complementarity” or “complementary” means that a nucleic acid can form hydrogen bond(s) with another nucleic acid sequence by either traditional Watson-Crick or other non-traditional types of interaction. In reference to the present disclosure, the binding free energy for a siRNA molecule with its complementary sequence is sufficient to allow the relevant function of the nucleic acid to proceed, e.g., RNAi activity. For example, the degree of complementarity between the sense and antisense strand of the siRNA molecule can be the same or different from the degree of complementarity between the antisense strand of the siRNA and the target RNA sequence.

A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. Preferably the term “complementarity” or “complementary” means that at least 90%, more preferably at least 95% and most preferably 100% of residues in a first nucleic acid sense can form hydrogen binds with a second nucleic acid sequence.

Complementary nucleic acid sequences hybridize under appropriate conditions to form stable duplexes containing few (one or two) or no mismatches. Furthermore, the sense strand and antisense strand of the siRNA can form a double stranded nucleotide or hairpin loop structure by the hybridization. In a preferred embodiment, such duplexes contain no more than 1 mismatch for every 10 matches. In an especially preferred embodiment, the sense and antisense strands of the duplex are fully complementary, i.e., the duplexes contain no mismatches.

As used herein, the term “cell” is defined using its usual biological sense. The cell can be present in an organism, e.g., mammals such as humans, cows, sheep, apes, monkeys, swine, dogs, and cats. The cell can be eukaryotic (e.g., a mammalian cell). The cell can be of somatic or germ line origin, totipotent or pluripotent, dividing or non-dividing. The cell can also be derived from or can comprise a gamete or embryo, a stem cell, or a fully differentiated cell. Preferably the cell is in the upper respiratory tract, pulmonary parenchyma, brain, colon, head and neck, kidney, liver, lung, or lymph.

As used herein, the term “RNA” means a molecule comprising at least one ribonucleotide residue. By “ribonucleotide” is meant a nucleotide with a hydroxyl group at the 2′ position of a beta-D-ribo-furanose moiety. The term includes double stranded RNA, single stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or internally, for example at one or more nucleotides of the RNA. Nucleotides in the RNA molecules of the instant disclosure can also comprise non-standard nucleotides, such as non-naturally occurring nucleotides or chemically synthesized nucleotides or deoxynucleotides. These altered RNAs can be referred to as analogues of naturally-occurring RNA. Preferably the term “RNA” consists of ribonucleotide residues only.

As used herein, the term“organism” refers to any living entity comprised of at least one cell. A living organism can be as simple as, for example, a single eukaryotic cell or as complex as a mammal, including a human being.

As used herein, the term “subject” means an organism, which is a donor or recipient of explanted cells or the cells themselves. “Subject” also refers to an organism to which the nucleic acid molecules of the disclosure can be administered. The subject is preferably a mammal, e.g., a human, non-human primate, mouse, rat, dog, cat, horse, or cow. Most preferably the subject is a human.

As used herein, the term “biological sample” refers to any sample containing polynucleotides. The sample may be a tissue or cell sample, or a body fluid containing polynucleotides (e.g., blood, mucus, lymphatic fluid, synovial fluid, cerebrospinal fluid, saliva, amniotic fluid, amniotic cord blood, urine, vaginal fluid and semen). The sample may be a homogenate, lysate, extract, cell culture or tissue culture prepared from a whole organism or a subset of its cells, tissues or component parts, or a fraction or portion thereof. Lastly, the sample may be a medium, such as a nutrient broth or gel in which an organism, or cells of an organism, have been propagated, wherein the sample contains polynucleotides.

The disclosure relates to methods of inhibiting spike (S) glycoprotein gene expression so that the protein product of the spike (S) glycoprotein from SARS-CoV-2 gene is not produced or is produced in reduced amounts. In particular, the disclosure provides a method for can be used to alter gene expression in a cell in which expression of spike (S) glycoprotein from SARS-CoV-2 is initiated, e.g., as a result of SARS-CoV-2-inflicted infectious conditions such as in asymptomatic infection, mild upper respiratory tract illness, severe viral pneumonia and with respiratory failure. Binding of the siRNA to a spike (S) glycoprotein mRNA transcript in a cell results in a reduction in spike (S) glycoprotein production by the infected cell. The cell may be further contacted with a transfection-enhancing agent to enhance delivery of the siRNA or siRNA encoding vector to the cell. Depending on the specific method of the present disclosure, the cell may be provided in vitro, in vivo or ex vivo.

Sequence information regarding the coronavirus SARS-CoV-2 spike (S) glycoprotein gene (GenBank accession NM_908947) was extracted from the NCBI Entrez nucleotide database. Up to 399 mRNA segments were identified. See for example, U.S. Pat. No. 6,506,559, and Elbashir et al., 2001, herein incorporated by reference in its entirety.

Selection of siRNA target sites can be performed as follows:
i) Beginning with the ATG start codon of the transcript, scan downstream for AA dinucleotide sequences. Record the occurrence of each AA and the 3′ adjacent 19 nucleotides as potential siRNA target sites. Tuschl et al. recommend against designing siRNA to the 5′ and 3′ untranslated regions (UTRs) and regions near the start codon (within 75 bases) as these may be richer in regulatory protein binding sites. UTR-binding proteins and/or translation initiation complexes may interfere with binding of the siRNA endonuclease complex.
ii) Compare the potential target sites to the appropriate genome database (human, mouse, rat, etc.) and eliminate from consideration any target sequences with significant homology to other coding sequences. We suggest using BLAST, which can be found on the NCBI server at: www.ncbi.nlm.nih.gov/BLAST/
iii) Select qualifying target sequences (i.e., sequences having over 45% GC content) for synthesis.

In one aspect of the disclosure, the length of the sense nucleic acid is at least 10 nucleotides and may be as long as the naturally-occurring spike (S) glycoprotein transcript. Preferably, the sense nucleic acid is less than 75, 50, or 25 nucleotides in length. It is further preferred that the sense nucleic acid comprises at least 19 nucleotides. Most preferably, the sense nucleic acid is 19-25 nucleotides in length. Examples of spike (S) glycoprotein from SARS-CoV-2 target siRNA sense nucleic acids of the present disclosure which inhibit spike (S) glycoprotein expression in mammalian cells include oligonucleotides comprising any one of the following target sequences of the spike (S) glycoprotein gene: SEQ ID No 35, SEQ ID No 36, SEQ ID No 113, SEQ ID No 114, SEQ ID No 161, SEQ ID No 162, SEQ ID No 181, SEQ ID No 217, SEQ ID No 223, SEQ ID No 224, SEQ ID No 226, SEQ ID No 227, SEQ ID No 230, SEQ ID No 231, SEQ ID No 303, SEQ ID No 304, SEQ ID No 305, SEQ ID No 307, SEQ ID No 308, SEQ ID No 309, SEQ ID No 327, SEQ ID No 328, SEQ ID No 329, SEQ ID No 332, SEQ ID No 333, SEQ ID No 337 or SEQ ID No 338.

Three hundred and forty-seven sequences, which set forth the sequence for one strand of the double stranded is RNA, were identified and isolated for spike (S) glycoprotein from SARS-CoV-2 (Table 1).

TABLE 1
5′ sense SARS-CoV-2 DNA target spike
(S) glycoprotein.

| SEQ No | 5′DNA sense |
|---|---|
| SEQ ID No 1 | AAAGATTGCTGATTATAATTA |
| SEQ ID No 2 | AAGATTGCTGATTATAATTAT |
| SEQ ID No 3 | AAGGTTGGTGGTAATTATAAT |
| SEQ ID No 4 | AAGTATGAGCAGTATATAAAA |
| SEQ ID No 5 | AGTGTCTCCTACTAAATTAAA |
| SEQ ID No 6 | GATTGCTGATTATAATTATAA |
| SEQ ID No 7 | ATTGCTGATTATAATTATAAA |
| SEQ ID No 8 | TAAGGTTGGTGGTAATTATAA |
| SEQ ID No 9 | AAGTATGAGCAGTATATAAAA |
| SEQ ID No 10 | AATCCTTCACTGTAGAAAAAG |
| SEQ ID No 11 | AACACCAGGAACAAATACTTC |
| SEQ ID No 12 | AAAACACCACCAATTAAAGAT |
| SEQ ID No 13 | AAACACCACCAATTAAAGATT |
| SEQ ID No 14 | AATCTTGCTGCTACTAAAATG |
| SEQ ID No 15 | AAGGAGGAGTTAGATAAATAT |
| SEQ ID No 16 | AAAGTATGAGCAGTATATAAA |
| SEQ ID No 17 | AAAGGAGTCAAATTACATTAC |
| SEQ ID No 18 | CTGAGAAGTCTAACATAATAA |
| SEQ ID No 19 | CGCTACTAATGTTGTTATTAA |
| SEQ ID No 20 | GCTACTAATGTTGTTATTAAA |
| SEQ ID No 21 | TTCTATTAAAATATAATGAAA |
| SEQ ID No 22 | TCTATTAAAATATAATGAAAA |
| SEQ ID No 23 | GAGTGTCTCCTACTAAATTAA |
| SEQ ID No 24 | TGGAAAGATTGCTGATTATAA |
| SEQ ID No 25 | TCTTACTGAGTCTAACAAAAA |
| SEQ ID No 26 | AGTGCTATTGGCAAAATTCAA |
| SEQ ID No 27 | CATATGTGACTCAACAATTAA |
| SEQ ID No 28 | GTGTACTTGGACAATCAAAAA |
| SEQ ID No 29 | TTCAAGGAGGAGTTAGATAAA |
| SEQ ID No 30 | TCAGTTGTAAACATTCAAAAA |
| SEQ ID No 31 | TTGTAAACATTCAAAAAGAAA |
| SEQ ID No 32 | GAAAGTATGAGCAGTATATAA |
| SEQ ID No 33 | AAAGTATGAGCAGTATATAAA |
| SEQ ID No 34 | AAGACCCAGTCCCTACTTATTGTTA |
| SEQ ID No 35 | AGTCCCTACTTATTGTTAA |
| SEQ ID No 36 | AGTCCCTACTTATTGTTAATAA |
| SEQ ID No 37 | CCCAGTCCCTACTTATTGTTAATAA |
| SEQ ID No 38 | AATAACGCTACTAATGTTGTT |
| SEQ ID No 39 | AACGCTACTAATGTTGTTATT |
| SEQ ID No 40 | AAAATATAATGAAAATGGAAC |
| SEQ ID No 41 | AAAATGGAACCATTACAGATG |
| SEQ ID No 42 | AACAAAGTGTACGTTGAAATC |
| SEQ ID No 43 | AAGTGTACGTTGAAATCCTTC |
| SEQ ID No 44 | AAATCCTTCACTGTAGAAAAA |
| SEQ ID No 45 | AAAAAGGAATCTATCAAACTT |
| SEQ ID No 46 | AAAAGGAATCTATCAAACTTC |
| SEQ ID No 47 | AAGGAATCTATCAAACTTCTA |
| SEQ ID No 48 | AATCTATCAAACTTCTAACTT |
| SEQ ID No 49 | AACCAACAGAATCTATTGTTA |
| SEQ ID No 50 | AACAGAATCTATTGTTAGATT |
| SEQ ID No 51 | AACTGTGTTGCTGATTATTCT |
| SEQ ID No 52 | AACCATACAGAGTAGTAGTAC |
| SEQ ID No 53 | AACATGTCAACAACTCATATG |
| SEQ ID No 54 | AAATTCAGTTGCTTACTCTAA |
| SEQ ID No 55 | AATTCAGTTGCTTACTCTAAT |
| SEQ ID No 56 | AAATGATTGCTCAATACACTT |
| SEQ ID No 57 | AATGATTGCTCAATACACTTC |
| SEQ ID No 58 | AAGTGCACTTGGAAAACTTCA |
| SEQ ID No 59 | AAGATGTGGTCAACCAAAATG |
| SEQ ID No 60 | AAACACGCTTGTTAAACAACT |
| SEQ ID No 61 | AACACGCTTGTTAAACAACTT |
| SEQ ID No 62 | AATTAGAGCTGCAGAAATCAG |
| SEQ ID No 63 | AACCACAAATCATTACTACAG |
| SEQ ID No 64 | AATGCTTCAGTTGTAAACATT |
| SEQ ID No 65 | AAACATTCAAAAAGAAATTGA |
| SEQ ID No 66 | AACATTCAAAAAGAAATTGAC |
| SEQ ID No 67 | AAGGAGTCAAATTACATTACA |
| SEQ ID No 68 | GTCAGTGTGTTAATCTTACAA |
| SEQ ID No 69 | CCACTGAGAAGTCTAACATAA |
| SEQ ID No 70 | TTATTACCACAAAAACAACAA |
| SEQ ID No 71 | TATTACCACAAAAACAACAAA |
| SEQ ID No 72 | ATTACCACAAAAACAACAAAA |
| SEQ ID No 73 | TAAAATATAATGAAAATGGAA |
| SEQ ID No 74 | GAAATCCTTCACTGTAGAAAA |
| SEQ ID No 75 | AAATCCTTCACTGTAGAAAAA |
| SEQ ID No 76 | CCTTCACTGTAGAAAAAGGAA |
| SEQ ID No 77 | GTAGAAAAAGGAATCTATCAA |
| SEQ ID No 78 | TAGAAAAAGGAATCTATCAAA |
| SEQ ID No 79 | AGGAATCTATCAAACTTCTAA |
| SEQ ID No 80 | TGATTATTCTGTCCTATATAA |
| SEQ ID No 81 | CGTTATAGCTTGGAATTCTAA |
| SEQ ID No 82 | TATAGCTTGGAATTCTAACAA |
| SEQ ID No 83 | TTCTAACAATCTTGATTCTAA |
| SEQ ID No 84 | GTTCTTACTGAGTCTAACAAA |
| SEQ ID No 85 | TTCTTACTGAGTCTAACAAAA |
| SEQ ID No 86 | TTGGTGGTGTCAGTGTTATAA |
| SEQ ID No 87 | ACCAGGAACAAATACTTCTAA |
| SEQ ID No 88 | AAATTCAGTTGCTTACTCTAA |
| SEQ ID No 89 | TTCAGTTGCTTACTCTAATAA |
| SEQ ID No 90 | AGACATCAGTAGATTGTACAA |
| SEQ ID No 91 | TAGCTGTTGAACAAGACAAAA |
| SEQ ID No 92 | AGCTGTTGAACAAGACAAAAA |
| SEQ ID No 93 | TTACAAAACACCACCAATTAA |
| SEQ ID No 94 | TACAAAACACCACCAATTAAA |
| SEQ ID No 95 | GCAGATGCTGGCTTCATCAAA |
| SEQ ID No 96 | GATGCTGGCTTCATCAAACAA |
| SEQ ID No 97 | GTGCAGGTGCTGCATTACAAA |
| SEQ ID No 98 | GTTCTCTATGAGAACCAAAAA |
| SEQ ID No 99 | AGTGCACTTGGAAAACTTCAA |
| SEQ ID No 100 | TTAAACACGCTTGTTAAACAA |
| SEQ ID No 101 | CTAATCTTGCTGCTACTAAAA |
| SEQ ID No 102 | GTGTGTACTTGGACAATCAAA |
| SEQ ID No 103 | TGTGTACTTGGACAATCAAAA |
| SEQ ID No 104 | CTGGTAACTGTGATGTTGTAA |
| SEQ ID No 105 | ACTGTGATGTTGTAATAGGAA |
| SEQ ID No 106 | TGTTGTAATAGGAATTGTCAA |
| SEQ ID No 107 | ATTCAAGGAGGAGTTAGATAA |
| SEQ ID No 108 | CATTAATGCTTCAGTTGTAAA |
| SEQ ID No 109 | GCTTCAGTTGTAAACATTCAA |
| SEQ ID No 110 | CTTCAGTTGTAAACATTCAAA |
| SEQ ID No 111 | TTCAGTTGTAAACATTCAAAA |
| SEQ ID No 112 | GTTGTAAACATTCAAAAAGAA |
| SEQ ID No 113 | AAGACCCAGTCCCTACTTATT |
| SEQ ID No 114 | AACCTGAATTAGACTCATT |
| SEQ ID No 115 | AATCTTACAACCAGAACTCAA |
| SEQ ID No 116 | AACTCAGGACTTGTTCTTACC |
| SEQ ID No 117 | AAGTCTAACATAATAAGAGGC |
| SEQ ID No 118 | AATGTTGTTATTAAAGTCTGT |
| SEQ ID No 119 | AAAAACAACAAAAGTTGGATG |
| SEQ ID No 120 | AACAAAAGTTGGATGGAAAGT |
| SEQ ID No 121 | AAAAGTTGGATGGAAAGTGAG |
| SEQ ID No 122 | AAAGTTGGATGGAAAGTGAGT |
| SEQ ID No 123 | AAGTTGGATGGAAAGTGAGTT |
| SEQ ID No 124 | AATAGGTATTAACATCACTAG |
| SEQ ID No 125 | AAATATAATGAAAATGGAACC |
| SEQ ID No 126 | AATGAAAATGGAACCATTACA |
| SEQ ID No 127 | AAATGGAACCATTACAGATGC |
| SEQ ID No 128 | AAACAAAGTGTACGTTGAAAT |
| SEQ ID No 129 | AAAGTGTACGTTGAAATCCTT |
| SEQ ID No 130 | AAAGGAATCTATCAAACTTCT |
| SEQ ID No 131 | AACAGGAAGAGAATCAGCAAC |
| SEQ ID No 132 | AAGAGAATCAGCAACTGTGTT |
| SEQ ID No 133 | AATTAGAGGTGATGAAGTCAG |
| SEQ ID No 134 | AAACTGGAAAGATTGCTGATT |
| SEQ ID No 135 | AACTGGAAAGATTGCTGATTA |
| SEQ ID No 136 | AATTCTAACAATCTTGATTCT |
| SEQ ID No 137 | AACAATCTTGATTCTAAGGTT |
| SEQ ID No 138 | AATTATAATTACCTGTATAGA |
| SEQ ID No 139 | AACTCATATGAGTGTGACATA |
| SEQ ID No 140 | AAAATTCAGTTGCTTACTCTA |
| SEQ ID No 141 | AAATTCTACCAGTGTCTATGA |
| SEQ ID No 142 | AATTCTACCAGTGTCTATGAC |
| SEQ ID No 143 | AAGACATCAGTAGATTGTACA |
| SEQ ID No 144 | AACTGGAATAGCTGTTGAACA |
| SEQ ID No 145 | AATAGCTGTTGAACAAGACAA |
| SEQ ID No 146 | AAATATTACCAGATCCATCAA |
| SEQ ID No 147 | AATATTACCAGATCCATCAAA |
| SEQ ID No 148 | AACAATATGGTGATTGCCTTG |
| SEQ ID No 149 | AATACACTTCTGCACTGTTAG |
| SEQ ID No 150 | AAGTGCAAATTGATAGGTTGA |
| SEQ ID No 151 | AAATTGATAGGTTGATCACAG |
| SEQ ID No 152 | AAATCAGAGCTTCTGCTAATC |
| SEQ ID No 153 | AATCAGAGCTTCTGCTAATCT |
| SEQ ID No 154 | AAAATGTCAGAGTGTGTACTT |
| SEQ ID No 155 | AAATGTCAGAGTGTGTACTTG |
| SEQ ID No 156 | AATGTCAGAGTGTGTACTTGG |
| SEQ ID No 157 | AAGAAAAGAACTTCACAACTG |
| SEQ ID No 158 | AATCATTACTACAGACAACAC |
| SEQ ID No 159 | AACTGTGATGTTGTAATAGGA |
| SEQ ID No 160 | AATAGGAATTGTCAACAACAC |
| SEQ ID No 161 | AACCTGAATTAGACTCATTCA |
| SEQ ID No 162 | AAATTGACCGCCTCAATGAGG |
| SEQ ID No 163 | AATGAATCTCTCATCGATCTC |
| SEQ ID No 164 | AATCTCTCATCGATCTCCAAG |
| SEQ ID No 165 | AAGAACTTGGAAAGTATGAGC |
| SEQ ID No 166 | AGTCTCTAGTCAGTGTGTTAA |
| SEQ ID No 167 | AATCTTACAACCAGAACTCAA |
| SEQ ID No 168 | TGCTTCCACTGAGAAGTCTAA |
| SEQ ID No 169 | TTCTTATGGACCTTGAAGGAA |
| SEQ ID No 170 | TCTTATGGACCTTGAAGGAAA |
| SEQ ID No 171 | CTTATGGACCTTGAAGGAAAA |
| SEQ ID No 172 | TTCTAAGCACACGCCTATTAA |
| SEQ ID No 173 | AGAAACAAAGTGTACGTTGAA |
| SEQ ID No 174 | GAAACAAAGTGTACGTTGAAA |
| SEQ ID No 175 | TTGAAATCCTTCACTGTAGAA |
| SEQ ID No 176 | TGAAATCCTTCACTGTAGAAA |
| SEQ ID No 177 | GAACAGGAAGAGAATCAGCAA |
| SEQ ID No 178 | TTATGGAGTGTCTCCTACTAA |
| SEQ ID No 179 | TATGGAGTGTCTCCTACTAAA |
| SEQ ID No 180 | GAGGTGATGAAGTCAGACAAA |
| SEQ ID No 181 | GGCCGGTAGCACACCTTGTAA |
| SEQ ID No 182 | TGGACCTAAAAAGTCTACTAA |
| SEQ ID No 183 | GGTTAAAAACAAATGTGTCAA |
| SEQ ID No 184 | AGGTGTTCTTACTGAGTCTAA |
| SEQ ID No 185 | TGTTCTTACTGAGTCTAACAA |
| SEQ ID No 186 | TGTTATAACACCAGGAACAAA |
| SEQ ID No 187 | TAGTTATCAGACTCAGACTAA |
| SEQ ID No 188 | TATGTCACTTGGTGCAGAAAA |
| SEQ ID No 189 | TCTACCAGTGTCTATGACCAA |
| SEQ ID No 190 | ACTGGAATAGCTGTTGAACAA |
| SEQ ID No 191 | AATAGCTGTTGAACAAGACAA |
| SEQ ID No 192 | ATAGCTGTTGAACAAGACAAA |
| SEQ ID No 193 | AAATATTACCAGATCCATCAA |
| SEQ ID No 194 | AATATTACCAGATCCATCAAA |
| SEQ ID No 195 | ATATTACCAGATCCATCAAAA |
| SEQ ID No 196 | TACCAGATCCATCAAAACCAA |
| SEQ ID No 197 | TGCAGATGCTGGCTTCATCAA |
| SEQ ID No 198 | ACAGATGAAATGATTGCTCAA |
| SEQ ID No 199 | GGTGCAGGTGCTGCATTACAA |
| SEQ ID No 200 | TTAATAGTGCTATTGGCAAAA |
| SEQ ID No 201 | CACAGCAAGTGCACTTGGAAA |
| SEQ ID No 202 | ACAGCAAGTGCACTTGGAAAA |
| SEQ ID No 203 | TTCAAGATGTGGTCAACCAAA |
| SEQ ID No 204 | TCAAGATGTGGTCAACCAAAA |
| SEQ ID No 205 | GTGGTCAACCAAAATGCACAA |
| SEQ ID No 206 | AAGTTGAGGCTGAAGTGCAAA |
| SEQ ID No 207 | TTGATCACAGGCAGACTTCAA |
| SEQ ID No 208 | TGATCACAGGCAGACTTCAAA |
| SEQ ID No 209 | CAGACATATGTGACTCAACAA |
| SEQ ID No 210 | AGAAATCAGAGCTTCTGCTAA |
| SEQ ID No 211 | TGCTAATCTTGCTGCTACTAA |
| SEQ ID No 212 | GCTAATCTTGCTGCTACTAAA |
| SEQ ID No 213 | AGTGTGTACTTGGACAATCAA |
| SEQ ID No 214 | CACAAGAAAAGAACTTCACAA |
| SEQ ID No 215 | ACAAATCATTACTACAGACAA |
| SEQ ID No 216 | TGTAATAGGAATTGTCAACAA |
| SEQ ID No 217 | ACCTGAATTAGACTCATTCAA |
| SEQ ID No 218 | AGGTGACATCTCTGGCATTAA |
| SEQ ID No 219 | GCATTAATGCTTCAGTTGTAA |
| SEQ ID No 220 | TCGATCTCCAAGAACTTGGAA |
| SEQ ID No 221 | CGATCTCCAAGAACTTGGAAA |
| SEQ ID No 222 | CAGTTGCTGTAGTTGTCTCAA |
| SEQ ID No 223 | CCGGTAGCACACCTTGTAA |
| SEQ ID No 224 | AGGCCGGTAGCACACCTTGTAA |
| SEQ ID No 225 | ATCAGGCCGGTAGCACACCTTGTAA |
| SEQ ID No 226 | CTGAATTAGACTCATTCAA |
| SEQ ID No 227 | AACCTGAATTAGACTCATTCAA |
| SEQ ID No 228 | TGCAACCTGAATTAGACTCATTCAA |
| SEQ ID No 229 | AACCTGAATTAGACTCATTCAAGGA |
| SEQ ID No 230 | AAATTGACCGCCTCAATGAG |
| SEQ ID No 231 | AATTGACCGCCTCAATGAG |
| SEQ ID No 232 | AATGTTACTTGGTTCCATGCT |
| SEQ ID No 233 | AAAACAACAAAAGTTGGATGG |
| SEQ ID No 234 | AAACAACAAAAGTTGGATGGA |
| SEQ ID No 235 | AACAACAAAAGTTGGATGGAA |
| SEQ ID No 236 | AAAATATATTCTAAGCACACG |
| SEQ ID No 237 | AATATAATGAAAATGGAACCA |
| SEQ ID No 238 | AATGGAACCATTACAGATGCT |
| SEQ ID No 239 | AACCATTACAGATGCTGTAGA |
| SEQ ID No 240 | AATCAGCAACTGTGTTGCTGA |
| SEQ ID No 241 | AATTATAAATTACCAGATGAT |
| SEQ ID No 242 | AATCTTGATTCTAAGGTTGGT |
| SEQ ID No 243 | AATGGTGTTGGTTACCAACCA |
| SEQ ID No 244 | AACAAATACTTCTAACCAGGT |
| SEQ ID No 245 | AACAACTCATATGAGTGTGAC |
| SEQ ID No 246 | AATCCATCATTGCCTACACTA |
| SEQ ID No 247 | AAAACCAAGCAAGAGGTCATT |
| SEQ ID No 248 | AACAAAGTGACACTTGCAGAT |
| SEQ ID No 249 | AAGTGACACTTGCAGATGCTG |
| SEQ ID No 250 | AAACAATATGGTGATTGCCTT |
| SEQ ID No 251 | AATCACTTCTGGTTGGACCTT |
| SEQ ID No 252 | AATGTTCTCTATGAGAACCAA |
| SEQ ID No 253 | AAAACTTCAAGATGTGGTCAA |
| SEQ ID No 254 | AAACTTCAAGATGTGGTCAAC |
| SEQ ID No 255 | AACTTCAAGATGTGGTCAACC |
| SEQ ID No 256 | AAAGTTGAGGCTGAAGTGCAA |
| SEQ ID No 257 | AATTGATAGGTTGATCACAGG |
| SEQ ID No 258 | AATTAATTAGAGCTGCAGAAA |
| SEQ ID No 259 | AAAAGAACTTCACAACTGCTC |
| SEQ ID No 260 | AAAGAACTTCACAACTGCTCC |
| SEQ ID No 261 | AAATCATTACTACAGACAACA |
| SEQ ID No 262 | AATTAGACTCATTCAAGGAGG |
| SEQ ID No 263 | AAGAATCATACATCACCAGAT |
| SEQ ID No 264 | AATCATACATCACCAGATGTT |
| SEQ ID No 265 | AAGAAATTGACCGCCTCAATG |
| SEQ ID No 266 | AATTGACCGCCTCAATGAGGT |
| SEQ ID No 267 | AAATGAATCTCTCATCGATCT |
| SEQ ID No 268 | AACTTGGAAAGTATGAGCAGT |
| SEQ ID No 269 | GTGTTAATCTTACAACCAGAA |
| SEQ ID No 270 | TATTGTTAATAACGCTACTAA |
| SEQ ID No 271 | GTTGTTATTAAAGTCTGTGAA |
| SEQ ID No 272 | AACAACAAAAGTTGGATGGAA |
| SEQ ID No 273 | ACAACAAAAGTTGGATGGAAA |
| SEQ ID No 274 | ATGCTTGGAACAGGAAGAGAA |
| SEQ ID No 275 | AGAGGTGATGAAGTCAGACAA |
| SEQ ID No 276 | AGGCTGCGTTATAGCTTGGAA |
| SEQ ID No 277 | TGATTCTAAGGTTGGTGGTAA |
| SEQ ID No 278 | ACACCTTGTAATGGTGTTGAA |
| SEQ ID No 279 | ACTAATGGTGTTGGTTACCAA |
| SEQ ID No 280 | GTGTTATAACACCAGGAACAA |
| SEQ ID No 281 | CAGGATGTTAACTGCACAGAA |
| SEQ ID No 282 | GTTGCTATTCATGCAGATCAA |
| SEQ ID No 283 | GCACGTAGTGTAGCTAGTCAA |
| SEQ ID No 284 | CTATGTCACTTGGTGCAGAAA |
| SEQ ID No 285 | ACTATTAGTGTTACCACAGAA |
| SEQ ID No 286 | CTATTAGTGTTACCACAGAAA |
| SEQ ID No 287 | TGATTCAACTGAATGCAGCAA |
| SEQ ID No 288 | TTAACTGGAATAGCTGTTGAA |
| SEQ ID No 289 | AGATCCATCAAAACCAAGCAA |
| SEQ ID No 290 | AATGTTCTCTATGAGAACCAA |
| SEQ ID No 291 | CCACAGCAAGTGCACTTGGAA |
| SEQ ID No 292 | AAAACTTCAAGATGTGGTCAA |
| SEQ ID No 293 | CTTCAAGATGTGGTCAACCAA |
| SEQ ID No 294 | TGTTAAACAACTTAGCTCCAA |
| SEQ ID No 295 | AAAGTTGAGGCTGAAGTGCAA |
| SEQ ID No 296 | TTGCAGACATATGTGACTCAA |
| SEQ ID No 297 | AATTAATTAGAGCTGCAGAAA |
| SEQ ID No 298 | TCAGAGTGTGTACTTGGACAA |
| SEQ ID No 299 | GAATCTCTCATCGATCTCCAA |
| SEQ ID No 300 | TCTCTCATCGATCTCCAAGAA |
| SEQ ID No 301 | TCTTGTGGATCCTGCTGCAAA |
| SEQ ID No 302 | CCAGTGCTCAAAGGAGTCAAA |
| SEQ ID No 303 | AAGACCCAGTCCCTACTTA |
| SEQ ID No 304 | TTGTTAATAACGCTACTAA |
| SEQ ID No 305 | TATTGTTAATAACGCTACTAA |
| SEQ ID No 306 | TACTTATTGTTAATAACGCTACTAA |
| SEQ ID No 307 | ACCTTGTAATGGTGTTGAA |
| SEQ ID No 308 | AACTGAAATCTATCAGGCC |
| SEQ ID No 309 | ACACCTTGTAATGGTGTTGAA |
| SEQ ID No 310 | TAGCACACCTTGTAATGGTGTTGAA |
| SEQ ID No 311 | AAATCTATCAGGCCGGTAGCACACC |
| SEQ ID No 312 | AAATTGACCGCCTCAATGAGGTTGC |
| SEQ ID No 313 | AAATATATTCTAAGCACACGC |
| SEQ ID No 314 | AAGTGTTATGGAGTGTCTCCT |
| SEQ ID No 315 | AACTTACTCCTACTTGGCGTG |
| SEQ ID No 316 | AAAGTGACACTTGCAGATGCT |
| SEQ ID No 317 | AATATGGTGATTGCCTTGGTG |
| SEQ ID No 318 | AACAATTAATTAGAGCTGCAG |
| SEQ ID No 319 | AAGAACTTCACAACTGCTCCT |
| SEQ ID No 320 | AAAGAAATTGACCGCCTCAAT |
| SEQ ID No 321 | AACTTCTACATGCACCAGCAA |
| SEQ ID No 322 | TCAGTGTTATAACACCAGGAA |
| SEQ ID No 323 | ACTATGTCACTTGGTGCAGAA |
| SEQ ID No 324 | CTTGACAAAGTTGAGGCTGAA |
| SEQ ID No 325 | CAATTAATTAGAGCTGCAGAA |
| SEQ ID No 326 | TTCTTGTGGATCCTGCTGCAA |
| SEQ ID No 327 | AATCTATCAGGCCGGTAGCAC |
| SEQ ID No 328 | AAATCTATCAGGCCGGTAGCAC |
| SEQ ID No 329 | AATCTATCAGGCCGGTAGCACA |
| SEQ ID No 330 | AATCTATCAGGCCGGTAGCACACCT |
| SEQ ID No 331 | AATTGACCGCCTCAATGAGGTTGCC |
| SEQ ID No 332 | AACTGAAATCTATCAGGCCGG |
| SEQ ID No 333 | AAATCTATCAGGCCGGTAGCA |
| SEQ ID No 334 | AACTTCACAACTGCTCCTGCC |
| SEQ ID No 335 | AAAAGAAATTGACCGCCTCAA |
| SEQ ID No 336 | AAAAGAAATTGACCGCCTCAA |
| SEQ ID No 337 | AAATCTATCAGGCCGGTAG |
| SEQ ID No 338 | AATCTATCAGGCCGGTAGC |
| SEQ ID No 339 | AACTGAAATCTATCAGGCCGGTAGC |

The spike (S) glycoprotein from SARS-CoV-2gene specificity was confirmed by searching NCBI BlastN database. The siRNAs were chemically synthesized.

All of the purified siRNA duplexes were complexed with lipofectamine and added to the cells for up to 12 h in serum-free medium. Thereafter, cells were cultured for 72-96 h in serum-supplemented medium, which was replaced by serum-free medium 24 h before the experiments. A scrambled negative siRNA duplex was used as control.

The spike (S) glycoprotein-siRNA is directed to a single target spike (S) glycoprotein from SARS-CoV-2 gene sequence. Alternatively, the siRNA is directed to multiple target spike (S) glycoprotein gene sequences. For example, the composition contains spike (S) glycoprotein-siRNA directed to two, three, four, five or more spike (S) glycoprotein target sequences. By spike (S) glycoprotein target sequence is meant a nucleotide sequence that is identical to a portion of the spike (S) glycoprotein gene. The target sequence can include the 5′ untranslated (UT) region, the open reading frame (ORF) or the 3′ untranslated region of the SARS-CoV-2 spike (S) glycoprotein gene. Alternatively, the siRNA is a nucleic acid sequence complementary to an upstream or downstream modulator of spike (S) glycoprotein gene expression. Examples of upstream and downstream modulators include, a transcription factor that binds the spike (S) glycoprotein gene promoter, a kinase or phosphatase that interacts with the spike (S) glycoprotein polypeptide, a spike (S) glycoprotein promoter or enhance.

SARS-CoV-2 spike (S) glycoprotein-siRNA which hybridize to target mRNA decrease or inhibit production of the spike (S) glycoprotein polypeptide product encoded by the spike (S) glycoprotein gene by associating with the normally single-stranded mRNA transcript, thereby interfering with translation and thus, expression of the protein. Exemplary nucleic acid sequence for the production of spike (S) glycoprotein-siRNA include the sequences of nucleotides SEQ ID No 35, SEQ ID No 36, SEQ ID No 113, SEQ ID No 114, SEQ ID No 161, SEQ ID No 162, SEQ ID No 181, SEQ ID No 217, SEQ ID No 223, SEQ ID No 224, SEQ ID No 226, SEQ ID No 227, SEQ ID No 230, SEQ ID No 231, SEQ ID No 303, SEQ ID No 304, SEQ ID No 305, SEQ ID No 307, SEQ ID No 308, SEQ ID No 309, SEQ ID No 327, SEQ ID No 328, SEQ ID No 329, SEQ ID No 332, SEQ ID No 333, SEQ ID No 337 or SEQ ID No 338, as the target sequence. In a further embodiment, in order to enhance the inhibition activity of the siRNA, nucleotide “u” can be added to 3′ end of the antisense strand of the target sequence. Preferably at least 2, more preferably 2 to 10, and most preferably 2 to 5 u's are added. The added u's form single strand at the 3′ end of the antisense strand of the siRNA.

The spike (S) glycoprotein-siRNA can be directly introduced into the cells in a form that is capable of binding to the mRNA transcripts. Alternatively, a vector encoding the spike (S) glycoprotein-siRNA can be introduced into the cells.

A loop sequence consisting of an arbitrary nucleotide sequence can be located between the sense and antisense sequence in order to form a hairpin loop structure. Thus, the present disclosure also provides siRNA having the general formula 5′-[A]-[B]-[A′]-3′, wherein [A] is a ribonucleotide sequence corresponding to a target sequence of the spike (S) glycoprotein gene. Preferably [A] is a sequence selected from the group consisting of nucleotides SEQ ID No 35, SEQ ID No 36, SEQ ID No 113, SEQ ID No 114, SEQ ID No 161, SEQ ID No 162, SEQ ID No 181, SEQ ID No 217, SEQ ID No 223, SEQ ID No 224, SEQ ID No 226, SEQ ID No 227, SEQ ID No 230, SEQ ID No 231, SEQ ID No 303, SEQ ID No 304, SEQ ID No 305, SEQ ID No 307, SEQ ID No 308, SEQ ID No 309, SEQ ID No 327, SEQ ID No 328, SEQ ID No 329, SEQ ID No 332, SEQ ID No 333, SEQ ID No 337 or SEQ ID No 338; [B] is a ribonucleotide sequence consisting of 3 to 23 nucleotides; and [A′] is a ribonucleotide sequence consisting of the complementary sequence of [A]. The region [A] hybridizes to [A′], and then a loop consisting of region [B] is formed. The loop sequence may be preferably 3 to 23 nucleotide in length. Suitable loop sequences are described at http://www.ambion.com/techlib/tb/tb_506.html. Furthermore, loop sequence consisting of 23 nucleotides also provides active siRNA (Jacque et al., 2002).

In an embodiment, 5′ sense siRNA sequences against spike (S) glycoprotein from SARS-CoV-2 target sequences were identified. The 5′ anti-sense siRNA sequences against spike (S) glycoprotein from SARS-CoV-2 were then designed and produced. Sense and anti-sense siRNA sequences have a length of 19 to 25 nucleotides. Table 2 shows 5′ sense and anti-sense siRNA sequences against spike (S) glycoprotein from SARS-CoV-2. siRNA sequences have a length of 19 to 25 nucleotides.

TABLE 2
5′ sense and anti-sense siRNA sequences of
spike (S) glycoprotein from SARS-CoV-2 -
19 to 25 nucleotides.

| SEQ No | 5′RNA sense |
|---|---|
| SEQ ID No 340 | AAAGAUUGCUGAUUAUAAUUA |
| SEQ ID No 341 | AAGAUUGCUGAUUAUAAUUAU |
| SEQ ID No 342 | AAGGUUGGUGGUAAUUAUAAU |
| SEQ ID No 343 | AAGUAUGAGCAGUAUAUAAAA |
| SEQ ID No 344 | AGUGUCUCCUACUAAAUUAAA |
| SEQ ID No 345 | GAUUGCUGAUUAUAAUUAUAA |
| SEQ ID No 346 | AUUGCUGAUUAUAAUUAUAAA |
| SEQ ID No 347 | UAAGGUUGGUGGUAAUUAUAA |
| SEQ ID No 348 | AAGUAUGAGCAGUAUAUAAAA |
| SEQ ID No 349 | AAUCCUUCACUGUAGAAAAAG |
| SEQ ID No 350 | AACACCAGGAACAAAUACUUC |
| SEQ ID No 351 | AAAACACCACCAAUUAAAGAU |
| SEQ ID No 352 | AAACACCACCAAUUAAAGAUU |
| SEQ ID No 353 | AAUCUUGCUGCUACUAAAAUG |
| SEQ ID No 354 | AAGGAGGAGUUAGAUAAAUAU |
| SEQ ID No 355 | AAAGUAUGAGCAGUAUAUAAA |
| SEQ ID No 356 | AAAGGAGUCAAAUUACAUUAC |
| SEQ ID No 357 | CUGAGAAGUCUAACAUAAUAA |
| SEQ ID No 358 | CGCUACUAAUGUUGUUAUUAA |
| SEQ ID No 359 | GCUACUAAUGUUGUUAUUAAA |
| SEQ ID No 360 | UUCUAUUAAAAUAUAAUGAAA |
| SEQ ID No 361 | UCUAUUAAAAUAUAAUGAAAA |
| SEQ ID No 362 | GAGUGUCUCCUACUAAAUUAA |
| SEQ ID No 363 | UGGAAAGAUUGCUGAUUAUAA |
| SEQ ID No 364 | UCUUACUGAGUCUAACAAAAA |
| SEQ ID No 365 | AGUGCUAUUGGCAAAAUUCAA |
| SEQ ID No 366 | CAUAUGUGACUCAACAAUUAA |
| SEQ ID No 367 | GUGUACUUGGACAAUCAAAAA |
| SEQ ID No 368 | UUCAAGGAGGAGUUAGAUAAA |
| SEQ ID No 369 | UCAGUUGUAAACAUUCAAAAA |
| SEQ ID No 370 | UUGUAAACAUUCAAAAAGAAA |
| SEQ ID No 371 | GAAAGUAUGAGCAGUAUAUAA |
| SEQ ID No 372 | AAAGUAUGAGCAGUAUAUAAA |
| SEQ ID No 373 | AAGACCCAGUCCCUACUUAUUGUUA |
| SEQ ID No 374 | AGUCCCUACUUAUUGUUAA |
| SEQ ID No 375 | AGUCCCUACUUAUUGUUAAUAA |
| SEQ ID No 376 | CCCAGUCCCUACUUAUUGUUAAUAA |
| SEQ ID No 377 | AAUAACGCUACUAAUGUUGUU |
| SEQ ID No 378 | AACGCUACUAAUGUUGUUAUU |
| SEQ ID No 379 | AAAAUAUAAUGAAAAUGGAAC |
| SEQ ID No 380 | AAAAUGGAACCAUUACAGAUG |
| SEQ ID No 381 | AACAAAGUGUACGUUGAAAUC |
| SEQ ID No 382 | AAGUGUACGUUGAAAUCCUUC |
| SEQ ID No 383 | AAAUCCUUCACUGUAGAAAAA |
| SEQ ID No 384 | AAAAAGGAAUCUAUCAAACUU |
| SEQ ID No 385 | AAAAGGAAUCUAUCAAACUUC |
| SEQ ID No 386 | AAGGAAUCUAUCAAACUUCUA |
| SEQ ID No 387 | AAUCUAUCAAACUUCUAACUU |
| SEQ ID No 388 | AACCAACAGAAUCUAUUGUUA |
| SEQ ID No 389 | AACAGAAUCUAUUGUUAGAUU |
| SEQ ID No 390 | AACUGUGUUGCUGAUUAUUCU |
| SEQ ID No 391 | AACCAUACAGAGUAGUAGUAC |
| SEQ ID No 392 | AACAUGUCAACAACUCAUAUG |
| SEQ ID No 393 | AAAUUCAGUUGCUUACUCUAA |
| SEQ ID No 394 | AAUUCAGUUGCUUACUCUAAU |
| SEQ ID No 395 | AAAUGAUUGCUCAAUACACUU |
| SEQ ID No 396 | AAUGAUUGCUCAAUACACUUC |
| SEQ ID No 397 | AAGUGCACUUGGAAAACUUCA |
| SEQ ID No 398 | AAGAUGUGGUCAACCAAAAUG |
| SEQ ID No 399 | AAACACGCUUGUUAAACAACU |
| SEQ ID No 400 | AACACGCUUGUUAAACAACUU |
| SEQ ID No 401 | AAUUAGAGCUGCAGAAAUCAG |
| SEQ ID No 402 | AACCACAAAUCAUUACUACAG |
| SEQ ID No 403 | AAUGCUUCAGUUGUAAACAUU |
| SEQ ID No 404 | AAACAUUCAAAAAGAAAUUGA |
| SEQ ID No 405 | AACAUUCAAAAAGAAAUUGAC |
| SEQ ID No 406 | AAGGAGUCAAAUUACAUUACA |
| SEQ ID No 407 | GUCAGUGUGUUAAUCUUACAA |
| SEQ ID No 408 | CCACUGAGAAGUCUAACAUAA |
| SEQ ID No 409 | UUAUUACCACAAAAACAACAA |
| SEQ ID No 410 | UAUUACCACAAAAACAACAAA |
| SEQ ID No 411 | AUUACCACAAAAACAACAAAA |
| SEQ ID No 412 | UAAAAUAUAAUGAAAAUGGAA |
| SEQ ID No 413 | GAAAUCCUUCACUGUAGAAAA |
| SEQ ID No 414 | AAAUCCUUCACUGUAGAAAAA |
| SEQ ID No 415 | CCUUCACUGUAGAAAAAGGAA |
| SEQ ID No 416 | GUAGAAAAAGGAAUCUAUCAA |
| SEQ ID No 417 | UAGAAAAAGGAAUCUAUCAAA |
| SEQ ID No 418 | AGGAAUCUAUCAAACUUCUAA |
| SEQ ID No 419 | UGAUUAUUCUGUCCUAUAUAA |
| SEQ ID No 420 | CGUUAUAGCUUGGAAUUCUAA |
| SEQ ID No 421 | UAUAGCUUGGAAUUCUAACAA |
| SEQ ID No 422 | UUCUAACAAUCUUGAUUCUAA |
| SEQ ID No 423 | GUUCUUACUGAGUCUAACAAA |
| SEQ ID No 424 | UUCUUACUGAGUCUAACAAAA |
| SEQ ID No 425 | UUGGUGGUGUCAGUGUUAUAA |
| SEQ ID No 426 | ACCAGGAACAAAUACUUCUAA |
| SEQ ID No 427 | AAAUUCAGUUGCUUACUCUAA |
| SEQ ID No 428 | UUCAGUUGCUUACUCUAAUAA |
| SEQ ID No 429 | AGACAUCAGUAGAUUGUACAA |
| SEQ ID No 430 | UAGCUGUUGAACAAGACAAAA |
| SEQ ID No 431 | AGCUGUUGAACAAGACAAAAA |
| SEQ ID No 432 | UUACAAAACACCACCAAUUAA |
| SEQ ID No 433 | UACAAAACACCACCAAUUAAA |
| SEQ ID No 434 | GCAGAUGCUGGCUUCAUCAAA |
| SEQ ID No 435 | GAUGCUGGCUUCAUCAAACAA |
| SEQ ID No 436 | GUGCAGGUGCUGCAUUACAAA |
| SEQ ID No 437 | GUUCUCUAUGAGAACCAAAAA |
| SEQ ID No 438 | AGUGCACUUGGAAAACUUCAA |
| SEQ ID No 439 | UUAAACACGCUUGUUAAACAA |
| SEQ ID No 440 | CUAAUCUUGCUGCUACUAAAA |
| SEQ ID No 441 | GUGUGUACUUGGACAAUCAAA |
| SEQ ID No 442 | UGUGUACUUGGACAAUCAAAA |
| SEQ ID No 443 | CUGGUAACUGUGAUGUUGUAA |
| SEQ ID No 444 | ACUGUGAUGUUGUAAUAGGAA |
| SEQ ID No 445 | UGUUGUAAUAGGAAUUGUCAA |
| SEQ ID No 446 | AUUCAAGGAGGAGUUAGAUAA |
| SEQ ID No 447 | CAUUAAUGCUUCAGUUGUAAA |
| SEQ ID No 448 | GCUUCAGUUGUAAACAUUCAA |
| SEQ ID No 449 | CUUCAGUUGUAAACAUUCAAA |
| SEQ ID No 450 | UUCAGUUGUAAACAUUCAAAA |
| SEQ ID No 451 | GUUGUAAACAUUCAAAAAGAA |
| SEQ ID No 452 | AAGACCCAGUCCCUACUUAUU |
| SEQ ID No 453 | AACCUGAAUUAGACUCAUU |
| SEQ ID No 454 | AAUCUUACAACCAGAACUCAA |
| SEQ ID No 455 | AACUCAGGACUUGUUCUUACC |
| SEQ ID No 456 | AAGUCUAACAUAAUAAGAGGC |
| SEQ ID No 457 | AAUGUUGUUAUUAAAGUCUGU |
| SEQ ID No 458 | AAAAACAACAAAAGUUGGAUG |
| SEQ ID No 459 | AACAAAAGUUGGAUGGAAAGU |
| SEQ ID No 460 | AAAAGUUGGAUGGAAAGUGAG |
| SEQ ID No 461 | AAAGUUGGAUGGAAAGUGAGU |
| SEQ ID No 462 | AAGUUGGAUGGAAAGUGAGUU |
| SEQ ID No 463 | AAUAGGUAUUAACAUCACUAG |
| SEQ ID No 464 | AAAUAUAAUGAAAAUGGAACC |
| SEQ ID No 465 | AAUGAAAAUGGAACCAUUACA |
| SEQ ID No 466 | AAAUGGAACCAUUACAGAUGC |
| SEQ ID No 467 | AAACAAAGUGUACGUUGAAAU |
| SEQ ID No 468 | AAAGUGUACGUUGAAAUCCUU |
| SEQ ID No 469 | AAAGGAAUCUAUCAAACUUCU |
| SEQ ID No 470 | AACAGGAAGAGAAUCAGCAAC |
| SEQ ID No 471 | AAGAGAAUCAGCAACUGUGUU |
| SEQ ID No 472 | AAUUAGAGGUGAUGAAGUCAG |
| SEQ ID No 473 | AAACUGGAAAGAUUGCUGAUU |
| SEQ ID No 474 | AACUGGAAAGAUUGCUGAUUA |
| SEQ ID No 475 | AAUUCUAACAAUCUUGAUUCU |
| SEQ ID No 476 | AACAAUCUUGAUUCUAAGGUU |
| SEQ ID No 477 | AAUUAUAAUUACCUGUAUAGA |
| SEQ ID No 478 | AACUCAUAUGAGUGUGACAUA |
| SEQ ID No 479 | AAAAUUCAGUUGCUUACUCUA |
| SEQ ID No 480 | AAAUUCUACCAGUGUCUAUGA |
| SEQ ID No 481 | AAUUCUACCAGUGUCUAUGAC |
| SEQ ID No 482 | AAGACAUCAGUAGAUUGUACA |
| SEQ ID No 483 | AACUGGAAUAGCUGUUGAACA |
| SEQ ID No 484 | AAUAGCUGUUGAACAAGACAA |
| SEQ ID No 485 | AAAUAUUACCAGAUCCAUCAA |
| SEQ ID No 486 | AAUAUUACCAGAUCCAUCAAA |
| SEQ ID No 487 | AACAAUAUGGUGAUUGCCUUG |
| SEQ ID No 488 | AAUACACUUCUGCACUGUUAG |
| SEQ ID No 489 | AAGUGCAAAUUGAUAGGUUGA |
| SEQ ID No 490 | AAAUUGAUAGGUUGAUCACAG |
| SEQ ID No 491 | AAAUCAGAGCUUCUGCUAAUC |
| SEQ ID No 492 | AAUCAGAGCUUCUGCUAAUCU |
| SEQ ID No 493 | AAAAUGUCAGAGUGUGUACUU |
| SEQ ID No 494 | AAAUGUCAGAGUGUGUACUUG |
| SEQ ID No 495 | AAUGUCAGAGUGUGUACUUGG |
| SEQ ID No 496 | AAGAAAAGAACUUCACAACUG |
| SEQ ID No 497 | AAUCAUUACUACAGACAACAC |
| SEQ ID No 498 | AACUGUGAUGUUGUAAUAGGA |
| SEQ ID No 499 | AAUAGGAAUUGUCAACAACAC |
| SEQ ID No 500 | AACCUGAAUUAGACUCAUUCA |
| SEQ ID No 501 | AAAUUGACCGCCUCAAUGAGG |
| SEQ ID No 502 | AAUGAAUCUCUCAUCGAUCUC |
| SEQ ID No 503 | AAUCUCUCAUCGAUCUCCAAG |
| SEQ ID No 504 | AAGAACUUGGAAAGUAUGAGC |
| SEQ ID No 505 | AGUCUCUAGUCAGUGUGUUAA |
| SEQ ID No 506 | AAUCUUACAACCAGAACUCAA |
| SEQ ID No 507 | UGCUUCCACUGAGAAGUCUAA |
| SEQ ID No 508 | UUCUUAUGGACCUUGAAGGAA |
| SEQ ID No 509 | UCUUAUGGACCUUGAAGGAAA |
| SEQ ID No 510 | CUUAUGGACCUUGAAGGAAAA |
| SEQ ID No 511 | UUCUAAGCACACGCCUAUUAA |
| SEQ ID No 512 | AGAAACAAAGUGUACGUUGAA |
| SEQ ID No 513 | GAAACAAAGUGUACGUUGAAA |
| SEQ ID No 514 | UUGAAAUCCUUCACUGUAGAA |
| SEQ ID No 515 | UGAAAUCCUUCACUGUAGAAA |
| SEQ ID No 516 | GAACAGGAAGAGAAUCAGCAA |
| SEQ ID No 517 | UUAUGGAGUGUCUCCUACUAA |
| SEQ ID No 518 | UAUGGAGUGUCUCCUACUAAA |
| SEQ ID No 519 | GAGGUGAUGAAGUCAGACAAA |
| SEQ ID No 520 | GGCCGGUAGCACACCUUGUAA |
| SEQ ID No 521 | UGGACCUAAAAAGUCUACUAA |
| SEQ ID No 522 | GGUUAAAAACAAAUGUGUCAA |
| SEQ ID No 523 | AGGUGUUCUUACUGAGUCUAA |
| SEQ ID No 524 | UGUUCUUACUGAGUCUAACAA |
| SEQ ID No 525 | UGUUAUAACACCAGGAACAAA |
| SEQ ID No 526 | UAGUUAUCAGACUCAGACUAA |
| SEQ ID No 527 | UAUGUCACUUGGUGCAGAAAA |
| SEQ ID No 528 | UCUACCAGUGUCUAUGACCAA |
| SEQ ID No 529 | ACUGGAAUAGCUGUUGAACAA |
| SEQ ID No 530 | AAUAGCUGUUGAACAAGACAA |
| SEQ ID No 531 | AUAGCUGUUGAACAAGACAAA |
| SEQ ID No 532 | AAAUAUUACCAGAUCCAUCAA |
| SEQ ID No 533 | AAUAUUACCAGAUCCAUCAAA |
| SEQ ID No 534 | AUAUUACCAGAUCCAUCAAAA |
| SEQ ID No 535 | UACCAGAUCCAUCAAAACCAA |
| SEQ ID No 536 | UGCAGAUGCUGGCUUCAUCAA |
| SEQ ID No 537 | ACAGAUGAAAUGAUUGCUCAA |
| SEQ ID No 538 | GGUGCAGGUGCUGCAUUACAA |
| SEQ ID No 539 | UUAAUAGUGCUAUUGGCAAAA |
| SEQ ID No 540 | CACAGCAAGUGCACUUGGAAA |
| SEQ ID No 541 | ACAGCAAGUGCACUUGGAAAA |
| SEQ ID No 542 | UUCAAGAUGUGGUCAACCAAA |
| SEQ ID No 543 | UCAAGAUGUGGUCAACCAAAA |
| SEQ ID No 544 | GUGGUCAACCAAAAUGCACAA |
| SEQ ID No 545 | AAGUUGAGGCUGAAGUGCAAA |
| SEQ ID No 546 | UUGAUCACAGGCAGACUUCAA |
| SEQ ID No 547 | UGAUCACAGGCAGACUUCAAA |
| SEQ ID No 548 | CAGACAUAUGUGACUCAACAA |
| SEQ ID No 549 | AGAAAUCAGAGCUUCUGCUAA |
| SEQ ID No 550 | UGCUAAUCUUGCUGCUACUAA |
| SEQ ID No 551 | GCUAAUCUUGCUGCUACUAAA |
| SEQ ID No 552 | AGUGUGUACUUGGACAAUCAA |
| SEQ ID No 553 | CACAAGAAAAGAACUUCACAA |
| SEQ ID No 554 | ACAAAUCAUUACUACAGACAA |
| SEQ ID No 555 | UGUAAUAGGAAUUGUCAACAA |
| SEQ ID No 556 | ACCUGAAUUAGACUCAUUCAA |
| SEQ ID No 557 | AGGUGACAUCUCUGGCAUUAA |
| SEQ ID No 558 | GCAUUAAUGCUUCAGUUGUAA |
| SEQ ID No 559 | UCGAUCUCCAAGAACUUGGAA |
| SEQ ID No 560 | CGAUCUCCAAGAACUUGGAAA |
| SEQ ID No 561 | CAGUUGCUGUAGUUGUCUCAA |
| SEQ ID No 562 | CCGGUAGCACACCUUGUAA |
| SEQ ID No 563 | AGGCCGGUAGCACACCUUGUAA |
| SEQ ID No 564 | AUCAGGCCGGUAGCACACCUUGUAA |
| SEQ ID No 565 | CUGAAUUAGACUCAUUCAA |
| SEQ ID No 566 | AACCUGAAUUAGACUCAUUCAA |
| SEQ ID No 567 | UGCAACCUGAAUUAGACUCAUUCAA |
| SEQ ID No 568 | AACCUGAAUUAGACUCAUUCAAGGA |
| SEQ ID No 569 | AAAUUGACCGCCUCAAUGAG |
| SEQ ID No 570 | AAUUGACCGCCUCAAUGAG |
| SEQ ID No 571 | AAUGUUACUUGGUUCCAUGCU |
| SEQ ID No 572 | AAAACAACAAAAGUUGGAUGG |
| SEQ ID No 573 | AAACAACAAAAGUUGGAUGGA |
| SEQ ID No 574 | AACAACAAAAGUUGGAUGGAA |
| SEQ ID No 575 | AAAAUAUAUUCUAAGCACACG |
| SEQ ID No 576 | AAUAUAAUGAAAAUGGAACCA |
| SEQ ID No 577 | AAUGGAACCAUUACAGAUGCU |
| SEQ ID No 578 | AACCAUUACAGAUGCUGUAGA |
| SEQ ID No 579 | AAUCAGCAACUGUGUUGCUGA |
| SEQ ID No 580 | AAUUAUAAAUUACCAGAUGAU |
| SEQ ID No 581 | AAUCUUGAUUCUAAGGUUGGU |
| SEQ ID No 582 | AAUGGUGUUGGUUACCAACCA |
| SEQ ID No 583 | AACAAAUACUUCUAACCAGGU |
| SEQ ID No 584 | AACAACUCAUAUGAGUGUGAC |
| SEQ ID No 585 | AAUCCAUCAUUGCCUACACUA |
| SEQ ID No 586 | AAAACCAAGCAAGAGGUCAUU |
| SEQ ID No 587 | AACAAAGUGACACUUGCAGAU |
| SEQ ID No 588 | AAGUGACACUUGCAGAUGCUG |
| SEQ ID No 589 | AAACAAUAUGGUGAUUGCCUU |
| SEQ ID No 590 | AAUCACUUCUGGUUGGACCUU |
| SEQ ID No 591 | AAUGUUCUCUAUGAGAACCAA |
| SEQ ID No 592 | AAAACUUCAAGAUGUGGUCAA |
| SEQ ID No 593 | AAACUUCAAGAUGUGGUCAAC |
| SEQ ID No 594 | AACUUCAAGAUGUGGUCAACC |
| SEQ ID No 595 | AAAGUUGAGGCUGAAGUGCAA |
| SEQ ID No 596 | AAUUGAUAGGUUGAUCACAGG |
| SEQ ID No 597 | AAUUAAUUAGAGCUGCAGAAA |
| SEQ ID No 598 | AAAAGAACUUCACAACUGCUC |
| SEQ ID No 599 | AAAGAACUUCACAACUGCUCC |
| SEQ ID No 600 | AAAUCAUUACUACAGACAACA |
| SEQ ID No 601 | AAUUAGACUCAUUCAAGGAGG |
| SEQ ID No 602 | AAGAAUCAUACAUCACCAGAU |
| SEQ ID No 603 | AAUCAUACAUCACCAGAUGUU |
| SEQ ID No 604 | AAGAAAUUGACCGCCUCAAUG |
| SEQ ID No 605 | AAUUGACCGCCUCAAUGAGGU |
| SEQ ID No 606 | AAAUGAAUCUCUCAUCGAUCU |
| SEQ ID No 607 | AACUUGGAAAGUAUGAGCAGU |
| SEQ ID No 608 | GUGUUAAUCUUACAACCAGAA |
| SEQ ID No 609 | UAUUGUUAAUAACGCUACUAA |
| SEQ ID No 610 | GUUGUUAUUAAAGUCUGUGAA |
| SEQ ID No 611 | AACAACAAAAGUUGGAUGGAA |
| SEQ ID No 612 | ACAACAAAAGUUGGAUGGAAA |
| SEQ ID No 613 | AUGCUUGGAACAGGAAGAGAA |
| SEQ ID No 614 | AGAGGUGAUGAAGUCAGACAA |
| SEQ ID No 615 | AGGCUGCGUUAUAGCUUGGAA |
| SEQ ID No 616 | UGAUUCUAAGGUUGGUGGUAA |
| SEQ ID No 617 | ACACCUUGUAAUGGUGUUGAA |
| SEQ ID No 618 | ACUAAUGGUGUUGGUUACCAA |
| SEQ ID No 619 | GUGUUAUAACACCAGGAACAA |
| SEQ ID No 620 | CAGGAUGUUAACUGCACAGAA |
| SEQ ID No 621 | GUUGCUAUUCAUGCAGAUCAA |
| SEQ ID No 622 | GCACGUAGUGUAGCUAGUCAA |
| SEQ ID No 623 | CUAUGUCACUUGGUGCAGAAA |
| SEQ ID No 624 | ACUAUUAGUGUUACCACAGAA |
| SEQ ID No 625 | CUAUUAGUGUUACCACAGAAA |
| SEQ ID No 626 | UGAUUCAACUGAAUGCAGCAA |
| SEQ ID No 627 | UUAACUGGAAUAGCUGUUGAA |
| SEQ ID No 628 | AGAUCCAUCAAAACCAAGCAA |
| SEQ ID No 629 | AAUGUUCUCUAUGAGAACCAA |
| SEQ ID No 630 | CCACAGCAAGUGCACUUGGAA |
| SEQ ID No 631 | AAAACUUCAAGAUGUGGUCAA |
| SEQ ID No 632 | CUUCAAGAUGUGGUCAACCAA |
| SEQ ID No 633 | UGUUAAACAACUUAGCUCCAA |
| SEQ ID No 634 | AAAGUUGAGGCUGAAGUGCAA |
| SEQ ID No 635 | UUGCAGACAUAUGUGACUCAA |
| SEQ ID No 636 | AAUUAAUUAGAGCUGCAGAAA |
| SEQ ID No 637 | UCAGAGUGUGUACUUGGACAA |
| SEQ ID No 638 | GAAUCUCUCAUCGAUCUCCAA |
| SEQ ID No 639 | UCUCUCAUCGAUCUCCAAGAA |
| SEQ ID No 640 | UCUUGUGGAUCCUGCUGCAAA |
| SEQ ID No 641 | CCAGUGCUCAAAGGAGUCAAA |
| SEQ ID No 642 | AAGACCCAGUCCCUACUUA |
| SEQ ID No 643 | UUGUUAAUAACGCUACUAA |
| SEQ ID No 644 | UAUUGUUAAUAACGCUACUAA |
| SEQ ID No 645 | UACUUAUUGUUAAUAACGCUACUAA |
| SEQ ID No 646 | ACCUUGUAAUGGUGUUGAA |
| SEQ ID No 647 | AACUGAAAUCUAUCAGGCC |
| SEQ ID No 648 | ACACCUUGUAAUGGUGUUGAA |
| SEQ ID No 649 | UAGCACACCUUGUAAUGGUGUUGAA |
| SEQ ID No 650 | AAAUCUAUCAGGCCGGUAGCACACC |
| SEQ ID No 651 | AAAUUGACCGCCUCAAUGAGGUUGC |
| SEQ ID No 652 | AAAUAUAUUCUAAGCACACGC |
| SEQ ID No 653 | AAGUGUUAUGGAGUGUCUCCU |
| SEQ ID No 654 | AACUUACUCCUACUUGGCGUG |
| SEQ ID No 655 | AAAGUGACACUUGCAGAUGCU |
| SEQ ID No 656 | AAUAUGGUGAUUGCCUUGGUG |
| SEQ ID No 657 | AACAAUUAAUUAGAGCUGCAG |
| SEQ ID No 658 | AAGAACUUCACAACUGCUCCU |
| SEQ ID No 659 | AAAGAAAUUGACCGCCUCAAU |
| SEQ ID No 660 | AACUUCUACAUGCACCAGCAA |
| SEQ ID No 661 | UCAGUGUUAUAACACCAGGAA |
| SEQ ID No 662 | ACUAUGUCACUUGGUGCAGAA |
| SEQ ID No 663 | CUUGACAAAGUUGAGGCUGAA |
| SEQ ID No 664 | CAAUUAAUUAGAGCUGCAGAA |
| SEQ ID No 665 | UUCUUGUGGAUCCUGCUGCAA |
| SEQ ID No 666 | AAUCUAUCAGGCCGGUAGCAC |
| SEQ ID No 667 | AAAUCUAUCAGGCCGGUAGCAC |
| SEQ ID No 668 | AAUCUAUCAGGCCGGUAGCACA |
| SEQ ID No 669 | AAUCUAUCAGGCCGGUAGCACACCU |
| SEQ ID No 670 | AAUUGACCGCCUCAAUGAGGUUGCC |
| SEQ ID No 671 | AACUGAAAUCUAUCAGGCCGG |
| SEQ ID No 672 | AAAUCUAUCAGGCCGGUAGCA |
| SEQ ID No 673 | AACUUCACAACUGCUCCUGCC |
| SEQ ID No 674 | AAAAGAAAUUGACCGCCUCAA |
| SEQ ID No 675 | AAAAGAAAUUGACCGCCUCAA |
| SEQ ID No 676 | AAAUCUAUCAGGCCGGUAG |
| SEQ ID No 677 | AAUCUAUCAGGCCGGUAGC |
| SEQ ID No 678 | AACUGAAAUCUAUCAGGCCGGUAGC |
| SEQ ID No 679 | UAAUUAUAAUCAGCAAUCUUU |
| SEQ ID No 680 | AUAAUUAUAAUCAGCAAUCUU |
| SEQ ID No 681 | AUUAUAAUUACCACCAACCUU |
| SEQ ID No 682 | UUUUAUAUACUGCUCAUACUU |
| SEQ ID No 683 | UUUAAUUUAGUAGGAGACACU |
| SEQ ID No 684 | UUAUAAUUAUAAUCAGCAAUC |
| SEQ ID No 685 | UUUAUAAUUAUAAUCAGCAAU |
| SEQ ID No 686 | UUAUAAUUACCACCAACCUUA |
| SEQ ID No 687 | UUUUAUAUACUGCUCAUACUU |
| SEQ ID No 688 | CUUUUUCUACAGUGAAGGAUU |
| SEQ ID No 689 | GAAGUAUUUGUUCCUGGUGUU |
| SEQ ID No 690 | AUCUUUAAUUGGUGGUGUUUU |
| SEQ ID No 691 | AAUCUUUAAUUGGUGGUGUUU |
| SEQ ID No 692 | CAUUUUAGUAGCAGCAAGAUU |
| SEQ ID No 693 | AUAUUUAUCUAACUCCUCCUU |
| SEQ ID No 694 | UUUAUAUACUGCUCAUACUUU |
| SEQ ID No 695 | GUAAUGUAAUUUGACUCCUUU |
| SEQ ID No 696 | UUAUUAUGUUAGACUUCUCAG |
| SEQ ID No 697 | UUAAUAACAACAUUAGUAGCG |
| SEQ ID No 698 | UUUAAUAACAACAUUAGUAGC |
| SEQ ID No 699 | UUUCAUUAUAUUUUAAUAGAA |
| SEQ ID No 700 | UUUUCAUUAUAUUUUAAUAGA |
| SEQ ID No 701 | UUAAUUUAGUAGGAGACACUC |
| SEQ ID No 702 | UUAUAAUCAGCAAUCUUUCCA |
| SEQ ID No 703 | UUUUUGUUAGACUCAGUAAGA |
| SEQ ID No 704 | UUGAAUUUUGCCAAUAGCACU |
| SEQ ID No 705 | UUAAUUGUUGAGUCACAUAUG |
| SEQ ID No 706 | UUUUUGAUUGUCCAAGUACAC |
| SEQ ID No 707 | UUUAUCUAACUCCUCCUUGAA |
| SEQ ID No 708 | UUUUUGAAUGUUUACAACUGA |
| SEQ ID No 709 | UUUCUUUUUGAAUGUUUACAA |
| SEQ ID No 710 | UUAUAUACUGCUCAUACUUUC |
| SEQ ID No 711 | UUUAUAUACUGCUCAUACUUU |
| SEQ ID No 712 | UAACAAUAAGUAGGGACUGGGUCUU |
| SEQ ID No 713 | UUAACAAUAAGUAGGGACU |
| SEQ ID No 714 | UUAUUAACAAUAAGUAGGGACU |
| SEQ ID No 715 | GGGUCAGGGAUGAAUAACAAUUAUU |
| SEQ ID No 716 | AACAACAUUAGUAGCGUUAUU |
| SEQ ID No 717 | AAUAACAACAUUAGUAGCGUU |
| SEQ ID No 718 | GUUCCAUUUUCAUUAUAUUUU |
| SEQ ID No 719 | CAUCUGUAAUGGUUCCAUUUU |
| SEQ ID No 720 | GAUUUCAACGUACACUUUGUU |
| SEQ ID No 721 | GAAGGAUUUCAACGUACACUU |
| SEQ ID No 722 | UUUUUCUACAGUGAAGGAUUU |
| SEQ ID No 723 | AAGUUUGAUAGAUUCCUUUUU |
| SEQ ID No 724 | GAAGUUUGAUAGAUUCCUUUU |
| SEQ ID No 725 | UAGAAGUUUGAUAGAUUCCUU |
| SEQ ID No 726 | AAGUUAGAAGUUUGAUAGAUU |
| SEQ ID No 727 | UAACAAUAGAUUCUGUUGGUU |
| SEQ ID No 728 | AAUCUAACAAUAGAUUCUGUU |
| SEQ ID No 729 | AGAAUAAUCAGCAACACAGUU |
| SEQ ID No 730 | GUACUACUACUCUGUAUGGUU |
| SEQ ID No 731 | CAUAUGAGUUGUUGACAUGUU |
| SEQ ID No 732 | UUAGAGUAAGCAACUGAAUUU |
| SEQ ID No 733 | AUUAGAGUAAGCAACUGAAUU |
| SEQ ID No 734 | AAGUGUAUUGAGCAAUCAUUU |
| SEQ ID No 735 | GAAGUGUAUUGAGCAAUCAUU |
| SEQ ID No 736 | UGAAGUUUUCCAAGUGCACUU |
| SEQ ID No 737 | CAUUUUGGUUGACCACAUCUU |
| SEQ ID No 738 | AGUUGUUUAACAAGCGUGUUU |
| SEQ ID No 739 | AAGUUGUUUAACAAGCGUGUU |
| SEQ ID No 740 | CUGAUUUCUGCAGCUCUAAUU |
| SEQ ID No 741 | CUGUAGUAAUGAUUUGUGGUU |
| SEQ ID No 742 | AAUGUUUACAACUGAAGCAUU |
| SEQ ID No 743 | UCAAUUUCUUUUUGAAUGUUU |
| SEQ ID No 744 | GUCAAUUUCUUUUUGAAUGUU |
| SEQ ID No 745 | UGUAAUGUAAUUUGACUCCUU |
| SEQ ID No 746 | UUGUAAGAUUAACACACUGAC |
| SEQ ID No 747 | UUAUGUUAGACUUCUCAGUGG |
| SEQ ID No 748 | UUGUUGUUUUUGUGGUAAUAA |
| SEQ ID No 749 | UUUGUUGUUUUUGUGGUAAUA |
| SEQ ID No 750 | UUUUGUUGUUUUUGUGGUAAU |
| SEQ ID No 751 | UUCCAUUUUCAUUAUAUUUUA |
| SEQ ID No 752 | UUUUCUACAGUGAAGGAUUUC |
| SEQ ID No 753 | UUUUUCUACAGUGAAGGAUUU |
| SEQ ID No 754 | UUCCUUUUUCUACAGUGAAGG |
| SEQ ID No 755 | UUGAUAGAUUCCUUUUUCUAC |
| SEQ ID No 756 | UUUGAUAGAUUCCUUUUUCUA |
| SEQ ID No 757 | UUAGAAGUUUGAUAGAUUCCU |
| SEQ ID No 758 | UUAUAUAGGACAGAAUAAUCA |
| SEQ ID No 759 | UUAGAAUUCCAAGCUAUAACG |
| SEQ ID No 760 | UUGUUAGAAUUCCAAGCUAUA |
| SEQ ID No 761 | UUAGAAUCAAGAUUGUUAGAA |
| SEQ ID No 762 | UUUGUUAGACUCAGUAAGAAC |
| SEQ ID No 763 | UUUUGUUAGACUCAGUAAGAA |
| SEQ ID No 764 | UUAUAACACUGACACCACCAA |
| SEQ ID No 765 | UUAGAAGUAUUUGUUCCUGGU |
| SEQ ID No 766 | UUAGAGUAAGCAACUGAAUUU |
| SEQ ID No 767 | UUAUUAGAGUAAGCAACUGAA |
| SEQ ID No 768 | UUGUACAAUCUACUGAUGUCU |
| SEQ ID No 769 | UUUUGUCUUGUUCAACAGCUA |
| SEQ ID No 770 | UUUUUGUCUUGUUCAACAGCU |
| SEQ ID No 771 | UUAAUUGGUGGUGUUUUGUAA |
| SEQ ID No 772 | UUUAAUUGGUGGUGUUUUGUA |
| SEQ ID No 773 | UUUGAUGAAGCCAGCAUCUGC |
| SEQ ID No 774 | UUGUUUGAUGAAGCCAGCAUC |
| SEQ ID No 775 | UUUGUAAUGCAGCACCUGCAC |
| SEQ ID No 776 | UUUUUGGUUCUCAUAGAGAAC |
| SEQ ID No 777 | UUGAAGUUUUCCAAGUGCACU |
| SEQ ID No 778 | UUGUUUAACAAGCGUGUUUAA |
| SEQ ID No 779 | UUUUAGUAGCAGCAAGAUUAG |
| SEQ ID No 780 | UUUGAUUGUCCAAGUACACAC |
| SEQ ID No 781 | UUUUGAUUGUCCAAGUACACA |
| SEQ ID No 782 | UUACAACAUCACAGUUACCAG |
| SEQ ID No 783 | UUCCUAUUACAACAUCACAGU |
| SEQ ID No 784 | UUGACAAUUCCUAUUACAACA |
| SEQ ID No 785 | UUAUCUAACUCCUCCUUGAAU |
| SEQ ID No 786 | UUUACAACUGAAGCAUUAAUG |
| SEQ ID No 787 | UUGAAUGUUUACAACUGAAGC |
| SEQ ID No 788 | UUUGAAUGUUUACAACUGAAG |
| SEQ ID No 789 | UUUUGAAUGUUUACAACUGAA |
| SEQ ID No 790 | UUCUUUUUGAAUGUUUACAAC |
| SEQ ID No 791 | AAUAAGUAGGGACUGGGUCUU |
| SEQ ID No 792 | AAUGAGUCUAAUUCAGGUU |
| SEQ ID No 793 | UUGAGUUCUGGUUGUAAGAUU |
| SEQ ID No 794 | GGUAAGAACAAGUCCUGAGUU |
| SEQ ID No 795 | GCCUCUUAUUAUGUUAGACUU |
| SEQ ID No 796 | ACAGACUUUAAUAACAACAUU |
| SEQ ID No 797 | CAUCCAACUUUUGUUGUUUUU |
| SEQ ID No 798 | ACUUUCCAUCCAACUUUUGUU |
| SEQ ID No 799 | CUCACUUUCCAUCCAACUUUU |
| SEQ ID No 800 | ACUCACUUUCCAUCCAACUUU |
| SEQ ID No 801 | AACUCACUUUCCAUCCAACUU |
| SEQ ID No 802 | CUAGUGAUGUUAAUACCUAUU |
| SEQ ID No 803 | GGUUCCAUUUUCAUUAUAUUU |
| SEQ ID No 804 | UGUAAUGGUUCCAUUUUCAUU |
| SEQ ID No 805 | GCAUCUGUAAUGGUUCCAUUU |
| SEQ ID No 806 | AUUUCAACGUACACUUUGUUU |
| SEQ ID No 807 | AAGGAUUUCAACGUACACUUU |
| SEQ ID No 808 | AGAAGUUUGAUAGAUUCCUUU |
| SEQ ID No 809 | GUUGCUGAUUCUCUUCCUGUU |
| SEQ ID No 810 | AACACAGUUGCUGAUUCUCUU |
| SEQ ID No 811 | CUGACUUCAUCACCUCUAAUU |
| SEQ ID No 812 | AAUCAGCAAUCUUUCCAGUUU |
| SEQ ID No 813 | UAAUCAGCAAUCUUUCCAGUU |
| SEQ ID No 814 | AGAAUCAAGAUUGUUAGAAUU |
| SEQ ID No 815 | AACCUUAGAAUCAAGAUUGUU |
| SEQ ID No 816 | UCUAUACAGGUAAUUAUAAUU |
| SEQ ID No 817 | UAUGUCACACUCAUAUGAGUU |
| SEQ ID No 818 | UAGAGUAAGCAACUGAAUUUU |
| SEQ ID No 819 | UCAUAGACACUGGUAGAAUUU |
| SEQ ID No 820 | GUCAUAGACACUGGUAGAAUU |
| SEQ ID No 821 | UGUACAAUCUACUGAUGUCUU |
| SEQ ID No 822 | UGUUCAACAGCUAUUCCAGUU |
| SEQ ID No 823 | UUGUCUUGUUCAACAGCUAUU |
| SEQ ID No 824 | UUGAUGGAUCUGGUAAUAUUU |
| SEQ ID No 825 | UUUGAUGGAUCUGGUAAUAUU |
| SEQ ID No 826 | CAAGGCAAUCACCAUAUUGUU |
| SEQ ID No 827 | CUAACAGUGCAGAAGUGUAUU |
| SEQ ID No 828 | UCAACCUAUCAAUUUGCACUU |
| SEQ ID No 829 | CUGUGAUCAACCUAUCAAUUU |
| SEQ ID No 830 | GAUUAGCAGAAGCUCUGAUUU |
| SEQ ID No 831 | AGAUUAGCAGAAGCUCUGAUU |
| SEQ ID No 832 | AAGUACACACUCUGACAUUUU |
| SEQ ID No 833 | CAAGUACACACUCUGACAUUU |
| SEQ ID No 834 | CCAAGUACACACUCUGACAUU |
| SEQ ID No 835 | CAGUUGUGAAGUUCUUUUCUU |
| SEQ ID No 836 | GUGUUGUCUGUAGUAAUGAUU |
| SEQ ID No 837 | UCCUAUUACAACAUCACAGUU |
| SEQ ID No 838 | GUGUUGUUGACAAUUCCUAUU |
| SEQ ID No 839 | UGAAUGAGUCUAAUUCAGGUU |
| SEQ ID No 840 | CCUCAUUGAGGCGGUCAAUUU |
| SEQ ID No 841 | GAGAUCGAUGAGAGAUUCAUU |
| SEQ ID No 842 | CUUGGAGAUCGAUGAGAGAUU |
| SEQ ID No 843 | GCUCAUACUUUCCAAGUUCUU |
| SEQ ID No 844 | UUAACACACUGACUAGAGACU |
| SEQ ID No 845 | UUGAGUUCUGGUUGUAAGAUU |
| SEQ ID No 846 | UUAGACUUCUCAGUGGAAGCA |
| SEQ ID No 847 | UUCCUUCAAGGUCCAUAAGAA |
| SEQ ID No 848 | UUUCCUUCAAGGUCCAUAAGA |
| SEQ ID No 849 | UUUUCCUUCAAGGUCCAUAAG |
| SEQ ID No 850 | UUAAUAGGCGUGUGCUUAGAA |
| SEQ ID No 851 | UUCAACGUACACUUUGUUUCU |
| SEQ ID No 852 | UUUCAACGUACACUUUGUUUC |
| SEQ ID No 853 | UUCUACAGUGAAGGAUUUCAA |
| SEQ ID No 854 | UUUCUACAGUGAAGGAUUUCA |
| SEQ ID No 855 | UUGCUGAUUCUCUUCCUGUUC |
| SEQ ID No 856 | UUAGUAGGAGACACUCCAUAA |
| SEQ ID No 857 | UUUAGUAGGAGACACUCCAUA |
| SEQ ID No 858 | UUUGUCUGACUUCAUCACCUC |
| SEQ ID No 859 | UUACAAGGUGUGCUACCGGCC |
| SEQ ID No 860 | UUAGUAGACUUUUUAGGUCCA |
| SEQ ID No 861 | UUGACACAUUUGUUUUUAACC |
| SEQ ID No 862 | UUAGACUCAGUAAGAACACCU |
| SEQ ID No 863 | UUGUUAGACUCAGUAAGAACA |
| SEQ ID No 864 | UUUGUUCCUGGUGUUAUAACA |
| SEQ ID No 865 | UUAGUCUGAGUCUGAUAACUA |
| SEQ ID No 866 | UUUUCUGCACCAAGUGACAUA |
| SEQ ID No 867 | UUGGUCAUAGACACUGGUAGA |
| SEQ ID No 868 | UUGUUCAACAGCUAUUCCAGU |
| SEQ ID No 869 | UUGUCUUGUUCAACAGCUAUU |
| SEQ ID No 870 | UUUGUCUUGUUCAACAGCUAU |
| SEQ ID No 871 | UUGAUGGAUCUGGUAAUAUUU |
| SEQ ID No 872 | UUUGAUGGAUCUGGUAAUAUU |
| SEQ ID No 873 | UUUUGAUGGAUCUGGUAAUAU |
| SEQ ID No 874 | UUGGUUUUGAUGGAUCUGGUA |
| SEQ ID No 875 | UUGAUGAAGCCAGCAUCUGCA |
| SEQ ID No 876 | UUGAGCAAUCAUUUCAUCUGU |
| SEQ ID No 877 | UUGUAAUGCAGCACCUGCACC |
| SEQ ID No 878 | UUUUGCCAAUAGCACUAUUAA |
| SEQ ID No 879 | UUUCCAAGUGCACUUGCUGUG |
| SEQ ID No 880 | UUUUCCAAGUGCACUUGCUGU |
| SEQ ID No 881 | UUUGGUUGACCACAUCUUGAA |
| SEQ ID No 882 | UUUUGGUUGACCACAUCUUGA |
| SEQ ID No 883 | UUGUGCAUUUUGGUUGACCAC |
| SEQ ID No 884 | UUUGCACUUCAGCCUCAACUU |
| SEQ ID No 885 | UUGAAGUCUGCCUGUGAUCAA |
| SEQ ID No 886 | UUUGAAGUCUGCCUGUGAUCA |
| SEQ ID No 887 | UUGUUGAGUCACAUAUGUCUG |
| SEQ ID No 888 | UUAGCAGAAGCUCUGAUUUCU |
| SEQ ID No 889 | UUAGUAGCAGCAAGAUUAGCA |
| SEQ ID No 890 | UUUAGUAGCAGCAAGAUUAGC |
| SEQ ID No 891 | UUGAUUGUCCAAGUACACACU |
| SEQ ID No 892 | UUGUGAAGUUCUUUUCUUGUG |
| SEQ ID No 893 | UUGUCUGUAGUAAUGAUUUGU |
| SEQ ID No 894 | UUGUUGACAAUUCCUAUUACA |
| SEQ ID No 895 | UUGAAUGAGUCUAAUUCAGGU |
| SEQ ID No 896 | UUAAUGCCAGAGAUGUCACCU |
| SEQ ID No 897 | UUACAACUGAAGCAUUAAUGC |
| SEQ ID No 898 | UUCCAAGUUCUUGGAGAUCGA |
| SEQ ID No 899 | UUUCCAAGUUCUUGGAGAUCG |
| SEQ ID No 900 | UUGAGACAACUACAGCAACUG |
| SEQ ID No 901 | UUACAAGGUGUGCUACCGG |
| SEQ ID No 902 | UUACAAGGUGUGCUACCGGCCU |
| SEQ ID No 903 | UUACAAGGUGUGCUACCGGCCUGAU |
| SEQ ID No 904 | UUGAAUGAGUCUAAUUCAG |
| SEQ ID No 905 | UUGAAUGAGUCUAAUUCAGGUU |
| SEQ ID No 906 | UUGAAUGAGUCUAAUUCAGGUUGCA |
| SEQ ID No 907 | UCCUUGAAUGAGUCUAAUUCAGGUU |
| SEQ ID No 908 | CUCAUUGAGGCGGUCAAUUU |
| SEQ ID No 909 | CUCAUUGAGGCGGUCAAUU |
| SEQ ID No 910 | AGCAUGGAACCAAGUAACAUU |
| SEQ ID No 911 | CCAUCCAACUUUUGUUGUUUU |
| SEQ ID No 912 | UCCAUCCAACUUUUGUUGUUU |
| SEQ ID No 913 | UUCCAUCCAACUUUUGUUGUU |
| SEQ ID No 914 | CGUGUGCUUAGAAUAUAUUUU |
| SEQ ID No 915 | UGGUUCCAUUUUCAUUAUAUU |
| SEQ ID No 916 | AGCAUCUGUAAUGGUUCCAUU |
| SEQ ID No 917 | UCUACAGCAUCUGUAAUGGUU |
| SEQ ID No 918 | UCAGCAACACAGUUGCUGAUU |
| SEQ ID No 919 | AUCAUCUGGUAAUUUAUAAUU |
| SEQ ID No 920 | ACCAACCUUAGAAUCAAGAUU |
| SEQ ID No 921 | UGGUUGGUAACCAACACCAUU |
| SEQ ID No 922 | ACCUGGUUAGAAGUAUUUGUU |
| SEQ ID No 923 | GUCACACUCAUAUGAGUUGUU |
| SEQ ID No 924 | UAGUGUAGGCAAUGAUGGAUU |
| SEQ ID No 925 | AAUGACCUCUUGCUUGGUUUU |
| SEQ ID No 926 | AUCUGCAAGUGUCACUUUGUU |
| SEQ ID No 927 | CAGCAUCUGCAAGUGUCACUU |
| SEQ ID No 928 | AAGGCAAUCACCAUAUUGUUU |
| SEQ ID No 929 | AAGGUCCAACCAGAAGUGAUU |
| SEQ ID No 930 | UUGGUUCUCAUAGAGAACAUU |
| SEQ ID No 931 | UUGACCACAUCUUGAAGUUUU |
| SEQ ID No 932 | GUUGACCACAUCUUGAAGUUU |
| SEQ ID No 933 | GGUUGACCACAUCUUGAAGUU |
| SEQ ID No 934 | UUGCACUUCAGCCUCAACUUU |
| SEQ ID No 935 | CCUGUGAUCAACCUAUCAAUU |
| SEQ ID No 936 | UUUCUGCAGCUCUAAUUAAUU |
| SEQ ID No 937 | GAGCAGUUGUGAAGUUCUUUU |
| SEQ ID No 938 | GGAGCAGUUGUGAAGUUCUUU |
| SEQ ID No 939 | UGUUGUCUGUAGUAAUGAUUU |
| SEQ ID No 940 | CCUCCUUGAAUGAGUCUAAUU |
| SEQ ID No 941 | AUCUGGUGAUGUAUGAUUCUU |
| SEQ ID No 942 | AACAUCUGGUGAUGUAUGAUU |
| SEQ ID No 943 | CAUUGAGGCGGUCAAUUUCUU |
| SEQ ID No 944 | ACCUCAUUGAGGCGGUCAAUU |
| SEQ ID No 945 | AGAUCGAUGAGAGAUUCAUUU |
| SEQ ID No 946 | ACUGCUCAUACUUUCCAAGUU |
| SEQ ID No 947 | UUCUGGUUGUAAGAUUAACAC |
| SEQ ID No 948 | UUAGUAGCGUUAUUAACAAUA |
| SEQ ID No 949 | UUCACAGACUUUAAUAACAAC |
| SEQ ID No 950 | UUCCAUCCAACUUUUGUUGUU |
| SEQ ID No 951 | UUUCCAUCCAACUUUUGUUGU |
| SEQ ID No 952 | UUCUCUUCCUGUUCCAAGCAU |
| SEQ ID No 953 | UUGUCUGACUUCAUCACCUCU |
| SEQ ID No 954 | UUCCAAGCUAUAACGCAGCCU |
| SEQ ID No 955 | UUACCACCAACCUUAGAAUCA |
| SEQ ID No 956 | UUCAACACCAUUACAAGGUGU |
| SEQ ID No 957 | UUGGUAACCAACACCAUUAGU |
| SEQ ID No 958 | UUGUUCCUGGUGUUAUAACAC |
| SEQ ID No 959 | UUCUGUGCAGUUAACAUCCUG |
| SEQ ID No 960 | UUGAUCUGCAUGAAUAGCAAC |
| SEQ ID No 961 | UUGACUAGCUACACUACGUGC |
| SEQ ID No 962 | UUUCUGCACCAAGUGACAUAG |
| SEQ ID No 963 | UUCUGUGGUAACACUAAUAGU |
| SEQ ID No 964 | UUUCUGUGGUAACACUAAUAG |
| SEQ ID No 965 | UUGCUGCAUUCAGUUGAAUCA |
| SEQ ID No 966 | UUCAACAGCUAUUCCAGUUAA |
| SEQ ID No 967 | UUGCUUGGUUUUGAUGGAUCU |
| SEQ ID No 968 | UUGGUUCUCAUAGAGAACAUU |
| SEQ ID No 969 | UUCCAAGUGCACUUGCUGUGG |
| SEQ ID No 970 | UUGACCACAUCUUGAAGUUUU |
| SEQ ID No 971 | UUGGUUGACCACAUCUUGAAG |
| SEQ ID No 972 | UUGGAGCUAAGUUGUUUAACA |
| SEQ ID No 973 | UUGCACUUCAGCCUCAACUUU |
| SEQ ID No 974 | UUGAGUCACAUAUGUCUGCAA |
| SEQ ID No 975 | UUUCUGCAGCUCUAAUUAAUU |
| SEQ ID No 976 | UUGUCCAAGUACACACUCUGA |
| SEQ ID No 977 | UUGGAGAUCGAUGAGAGAUUC |
| SEQ ID No 978 | UUCUUGGAGAUCGAUGAGAGA |
| SEQ ID No 979 | UUUGCAGCAGGAUCCACAAGA |
| SEQ ID No 980 | UUUGACUCCUUUGAGCACUGG |
| SEQ ID No 981 | UAAGUAGGGACUGGGUCUU |
| SEQ ID No 982 | UUAGUAGCGUUAUUAACAA |
| SEQ ID No 983 | UUAGUAGCGUUAUUAACAAUA |
| SEQ ID No 984 | UUAGUAGCGUUAUUAACAAUAAGUA |
| SEQ ID No 985 | UUCAACACCAUUACAAGGU |
| SEQ ID No 986 | GGCCUGAUAGAUUUCAGUU |
| SEQ ID No 987 | UUCAACACCAUUACAAGGUGU |
| SEQ ID No 988 | UUCAACACCAUUACAAGGUGUGCUA |
| SEQ ID No 989 | GGUGUGCUACCGGCCUGAUAGAUUU |
| SEQ ID No 990 | GCAACCUCAUUGAGGCGGUCAAUUU |
| SEQ ID No 991 | GCGUGUGCUUAGAAUAUAUUU |
| SEQ ID No 992 | AGGAGACACUCCAUAACACUU |
| SEQ ID No 993 | CACGCCAAGUAGGAGUAAGUU |
| SEQ ID No 994 | AGCAUCUGCAAGUGUCACUUU |
| SEQ ID No 995 | CACCAAGGCAAUCACCAUAUU |
| SEQ ID No 996 | CUGCAGCUCUAAUUAAUUGUU |
| SEQ ID No 997 | AGGAGCAGUUGUGAAGUUCUU |
| SEQ ID No 998 | AUUGAGGCGGUCAAUUUCUUU |
| SEQ ID No 999 | UUGCUGGUGCAUGUAGAAGUU |
| SEQ ID No 1000 | UUCCUGGUGUUAUAACACUGA |
| SEQ ID No 1001 | UUCUGCACCAAGUGACAUAGU |
| SEQ ID No 1002 | UUCAGCCUCAACUUUGUCAAG |
| SEQ ID No 1003 | UUCUGCAGCUCUAAUUAAUUG |
| SEQ ID No 1004 | UUGCAGCAGGAUCCACAAGAA |
| SEQ ID No 1005 | GUGCUACCGGCCUGAUAGAUU |
| SEQ ID No 1006 | GUGCUACCGGCCUGAUAGAUUU |
| SEQ ID No 1007 | UGUGCUACCGGCCUGAUAGAUU |
| SEQ ID No 1008 | AGGUGUGCUACCGGCCUGAUAGAUU |
| SEQ ID No 1009 | GGCAACCUCAUUGAGGCGGUCAAUU |
| SEQ ID No 1010 | CCGGCCUGAUAGAUUUCAGUU |
| SEQ ID No 1011 | UGCUACCGGCCUGAUAGAUUU |
| SEQ ID No 1012 | GGCAGGAGCAGUUGUGAAGUU |
| SEQ ID No 1013 | UUGAGGCGGUCAAUUUCUUUU |
| SEQ ID No 1014 | UUGAGGCGGUCAAUUUCUUUU |
| SEQ ID No 1015 | CUACCGGCCUGAUAGAUUU |
| SEQ ID No 1016 | GCUACCGGCCUGAUAGAUU |
| SEQ ID No 1017 | GCUACCGGCCUGAUAGAUUUCAGUU |

The inventors have surprisingly found that siRNAs targeted to certain target sequences of the SARS-CoV-2 spike (S) glycoprotein gene are particularly effective at inhibiting spike (S) glycoprotein mRNA expression, inhibiting spike (S) glycoprotein expression for virus-cell receptor interactions during viral entry into a cell, SARS-CoV-2 viral entry into a cell, and increase the survival of SARS-CoV-2 infected mice treated by intranasal administration of siRNAs targeting certain sequences of the SARS-CoV-2 spike (S) glycoprotein gene.

In a specific embodiment of the present disclosure, the sense strand of the SARS-CoV-2 spike (S) glycoprotein siRNA used in the present disclosure comprises or consists of a sequence selected from the group comprising SEQ ID No 340 to SEQ ID No 678, preferably SEQ ID No 374, SEQ ID No 375, SEQ ID No 452, SEQ ID No 453, SEQ ID No 500, SEQ ID No 501, SEQ ID No 520, SEQ ID No 556, SEQ ID No 562, SEQ ID No 563, SEQ ID No 565, SEQ ID No 566, SEQ ID No 569, SEQ ID No 570, SEQ ID No 642, SEQ ID No 643, SEQ ID No 644, SEQ ID No 646, SEQ ID No 647, SEQ ID No 648, SEQ ID No 666, SEQ ID No 667, SEQ ID No 668, SEQ ID No 671, SEQ ID No 672, SEQ ID No 676 or SEQ ID No 677, or a variant thereof. The siRNA also comprises a corresponding antisense strand comprising SEQ ID No 679 to SEQ ID No 1017, preferably SEQ ID No 713, SEQ ID No 714, SEQ ID No 791, SEQ ID No 792, SEQ ID No 839, SEQ ID No 840, SEQ ID No 859, SEQ ID No 895, SEQ ID No 901, SEQ ID No 902, SEQ ID No 904, SEQ ID No 905, SEQ ID No 908, SEQ ID No 909, SEQ ID No 981, SEQ ID No 982, SEQ ID No 983, SEQ ID No 985, SEQ ID No 986, SEQ ID No 987, SEQ ID No 1005, SEQ ID No 1006, SEQ ID No 1007, SEQ ID No 1010, SEQ ID No 1011, SEQ ID No 1015 or SEQ ID No 1016. The use of such an siRNA has been found to be particularly effective in inhibiting spike (S) glycoprotein mRNA expression, inhibiting spike (S) glycoprotein expression for virus-cell receptor interactions during viral entry into a cell, SARS-CoV-2 viral entry into a cell, and increase the survival of SARS-CoV-2 infected mice treated by intranasal administration of siRNAs targeting certain sequences of the SARS-CoV-2 spike (S) glycoprotein gene.

According to a another aspect of the present disclosure there is provided a siRNA comprising a sense SARS-CoV-2 spike (S) glycoprotein nucleic acid and an anti-sense SARS-CoV-2 spike (S) glycoprotein nucleic acid, and the sense SARS-CoV-2 spike (S) glycoprotein nucleic acid is substantially identical to a target sequence contained within SARS-CoV-2 spike (S) glycoprotein mRNA and the anti-sense SARS-CoV-2 spike (S) glycoprotein nucleic acid is complementary to the sense SARS-CoV-2 spike (S) glycoprotein nucleic acid. The sense and antisense nucleic acids hybridize to each other to form a double-stranded molecule.

The siRNA molecules of the present disclosure have the property to inhibit expression of the SARS-CoV-2 spike (S) glycoprotein gene when introduced into a cell expressing said gene.

The siRNA molecules of the present disclosure have the property to inhibit SARS-CoV-2 viral entry into a cell when introduced into a cell expressing SARS-CoV-2 spike (S) glycoprotein gene.

The siRNA molecules of the present disclosure have the property to increase the survival of SARS-CoV-2 infected mice treated by intranasal administration of siRNAs targeting certain sequences of the SARS-CoV-2 spike (S) glycoprotein gene.

Another aspect of the disclosure relates to nucleic acid sequences and vectors encoding the siRNA according to the fourth aspect of the present disclosure, as well as to compositions comprising them, useful, for example, in the methods of the present disclosure. Compositions of the present disclosure may additionally comprise transfection enhancing agents. The nucleic acid sequence may be operably linked to an inducible or regulatable promoter. Suitable vectors are discussed above. Preferably the vector is an adeno-associated viral vector.

The composition of the present disclosure may additionally comprise a pharmaceutical agent for preventing and treating infections by the coronavirus SARS-CoV-2, wherein the agent is different from the siRNA. Preferably the pharmaceutical agent is selected from the group consisting of a nucleoside analogue antiviral agent and most preferably favipiravir, ribavirin, remdesivir and galidesivir.

Non-viral delivery siRNA systems involve the creation of nucleic acid transfection reagents. Nucleic acid transfection reagents have two basic properties. First, they must interact in some manner with the nucleic acid cargo. Most often this involves electrostatic forces, which allow the formation of nucleic acid complexes. Formation of a complex ensures that the nucleic acid and transfection reagents are presented simultaneously to the cell membrane. Complexes can be divided into three classes, based on the nature of the delivery reagent: lipoplexes; polyplexes; and lipopolyplexes. Lipoplexes are formed by the interaction of anionic nucleic acids with cationic lipids, polyplexes by interaction with cationic polymers. Lipopolyplex reagents can combine the action of cationic lipids and polymers to deliver nucleic acids. Addition of histone, poly-L-lysine and protamine to some formulations of cationic lipids results in levels of delivery that are higher than either lipid or polymer alone. The combined formulations might also be less toxic. The biocompatible systems most relevant to this purpose are non-viral biodegradable nanocapsules designed especially according to the physical chemistry of nucleic acids. They have an aqueous core surrounded by a biodegradable polymeric envelope, which provides protection and transport of the siRNA into the cytosol and allow the siRNA to function efficiently in vivo.

The present disclosure also provides a cell containing the siRNA according to the fourth aspect of the present disclosure or the vector of the present disclosure. Preferably the cell is a mammalian cell, more preferably a human cell. It is further preferred that the cell is an isolated cell.

While the foregoing disclosure provides a general description of the subject matter encompassed within the scope of the present disclosure, including methods, as well as the best mode thereof, of making and using this disclosure, the following examples are provided to further enable those skilled in the art to practice this disclosure and to provide a complete written description thereof. However, those skilled in the art will appreciate that the specifics of these examples should not be read as limiting on the disclosure, the scope of which should be apprehended from the claims and equivalents thereof appended to this disclosure. Various further aspects and embodiments of the present disclosure will be apparent to those skilled in the art in view of the present disclosure.

All documents mentioned in this specification, including reference to sequence database identifiers, are incorporated herein by reference in their entirety. Unless otherwise specified, when reference to sequence database identifiers is made, the version number is 1.

Unless context dictates otherwise, the descriptions and definitions of the features set out above are not limited to any particular aspect or embodiment of the disclosure and apply equally to all aspects and embodiments which are described. The disclosure is further described in the following non-limiting examples.

The following examples further illustrate the present disclosure in detail but are not to be construed to limit the scope thereof.

### DESCRIPTION OF THE DRAWINGS

The following figures provide preferred embodiments for illustrating the disclosure and should not be seen as limiting the scope of invention.

FIG. 1. Integrity of a natural (siNACoV-1) or chemically modified (siNACoV-F1) 21 nucleotide siRNA anti-SARS-CoV-2 spike (S) glycoprotein when exposed for 30 min in cell culture medium in the absence (0%) and the presence of increasing amounts of serum (fetal bovine serum) (5% or 10%).

FIG. 2. Integrity of a natural (siNACoV-1) or chemically modified (siNACoV-F1) 21 nucleotide siRNA anti-SARS-CoV-2 spike (S) glycoprotein when exposed for 30 min (A and B) or 120 min (C) in cell culture medium in the absence and the presence of RNase I (0.25 or 0.50 Units).

FIG. 3. SARS-CoV-2 spike S2-GFP mRNA expression as determined by PCR after treatment with siRNA/transfection agent complexes. Values are shown as a % of RNAiMAX. siRNA/transfection agent complexes prepared with RNAiMAX at a final concentration of the 22 nucleotide siNACoV-2 (10 or 50 nM) siRNA anti-SARS-CoV-2 spike (S) glycoprotein or the negative control NC2 (S103650325, from Qiagen, Germany) at 48 h after treatment. Significantly different from corresponding control values (* P<0.001).

FIG. 4. Relative abundance of SARS-CoV-2 spike (S) glycoprotein mRNA in Vero 6E cells expressing SARS-CoV-2 spike (S) glycoprotein by RT-qPCR after exposure (6 h) to transfection agent (0.25% RNAiMAX) and 21 nucleotide siNACoV-1 (10 nM) siRNA anti-SARS-CoV-2 spike (S) glycoprotein at 84 h after treatment. Significantly different from corresponding control values (* P<0.001).

siNA molecules described in the present disclosure are tested in one or more of these examples and show to have activity and stability.

### EXAMPLE 1

Cell culture: Human embryonic kidney (HEK) (293T) cell line transiently transfected with a plasmid containing the SARS-CoV-2 spike Spike glycoprotein S2 subunit+GFP fusion gene (S2-GFP plasmid) (Sino Biological/VG40590-ACG) were maintained in a humidified atmosphere of 5% CO₂ at 37° C. Cells were grown in Dulbecco's Modified Eagle's Medium (Sigma, St. Louis, Mo.) supplemented with 10% fetal bovine serum (FBS) (Gibco, UK), 100 U/mL penicillin G, 0.25 μg/mL amphotericin B, 100 μg/mL streptomycin (Gibco, UK), 18 mM sodium bicarbonate (Merck, Germany) and 25 mM N-2-hydroxyethylpiperazine-N′-2-ethanosulfonic acid (HEPES) (Sigma, St. Louis, Mo.). The medium was changed every 2 days, and cells reached confluence 3-4 days after initial seeding. For subculturing, cells were dissociated with 0.25% trypsin-ethylenediaminetetraacetic acid (EDTA) (Sigma, St. Louis, Mo.), split 1:15 or 1:20 and subcultured in a 21-cm² growth area (Sarstedt, Germany).

### EXAMPLE 2

SARS-CoV-2 spike (S) glycoprotein gene silencing: Total RNA was isolated and purified using the SV Total RNA Isolation System (Promega, USA) according to manufacturer's instructions. RNA quality and concentration were verified in the NanoDrop ND1000 Spectrophotometer (Thermo Scientific, USA), and RNA integrity and genomic DNA contamination were evaluated by agarose gel electrophoresis. Total RNA (1 μg) was converted into cDNA using the Maxima Scientific First Strand cDNA Synthesis Kit for RT-qPCR (Thermo Scientific, USA), according to instructions. The following protocol was used: 1^{st} step, 10 min at 25° C.; 2^{nd} step, 15 min at 50° C.; 3^{rd} step, 5 min at 85° C. cDNA was used for qPCR analysis using Maxima SYBR Green qPCR Master Mix (Thermo Scientific, USA) in the StepOnePlus instrument (Applied Biosystems, USA). Primer Assay for SARS-CoV-2 and for the endogenous control gene GAPDH (Quiagen, Germany) were used. The qPCR reaction was performed in 96-well PCR plates (Sarstedt, Germany) as follows: one cycle of 10 min at 95° C., followed by 40 PCR cycles at 95° C. 15 s and 60° C. 60 s. A melting curve was made immediately after the qPCR, to demonstrate the specificity of the amplification. No template controls were always evaluated for each target gene. Quantification cycle (Cq) values were generated automatically by the StepOnePlus 2.3 Software and the ratio of the target gene was expressed in comparison to the endogenous control gene GAPDH. Real-time PCR efficiencies were found to be between 90% and 110%.

### EXAMPLE 3

SARS-CoV-2 spike (S) glycoprotein expression: Cells were rinsed twice with cold phosphate-buffered saline (PBS) and incubated with 100 μL RIPA lysis buffer (154 mM NaCl, 65.2 mM TRIZMA base, 1 mM EDTA, 1% NP-40 (IGEPAL), 6 mM sodium deoxycholate) containing protease inhibitors: 1 mM PMSF, 1 μg/mL leupeptine and 1 μg/mL aprotinin; and phosphatase inhibitors: 1 mM Na₃VO₄ and 1 mM NaF. Cells were scraped and briefly sonicated. Equal amounts of total protein (30 μg) were separated on a 10% SDS-polyacrylamide gel and electrotransfered to a nitrocellulose membrane in Tris-Glycine transfer buffer containing 20% methanol. The transblot sheets were blocked in 5% non-fat dry milk in Tris-buffered saline (TBS) for 60 min and then incubated overnight, at 4° C., with the antibodies against SARS-CoV-2 and GAPDH, diluted in 2.5% non-fat dry milk in TBS-Tween 20 (0.1% vol/vol). The immunoblots were subsequently washed and incubated with fluorescently-labelled secondary antibodies (1:20,000; AlexaFluor 680, Molecular Probes) for 60 min at room temperature (RT) and protected from light. Membranes were washed and imaged by scanning at both 700 nm and 800 nm with an Odyssey Infrared Imaging System (LI-COR Biosciences).

### EXAMPLE 4

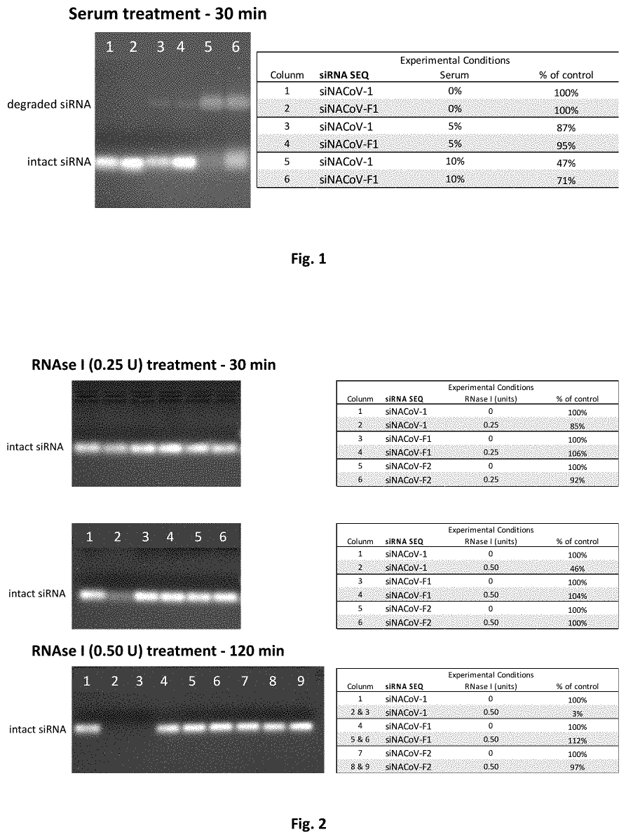

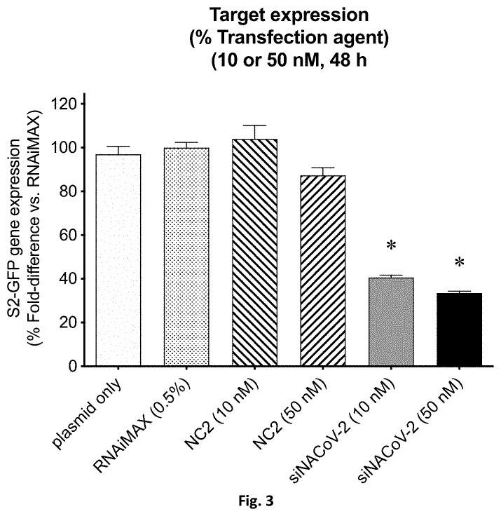

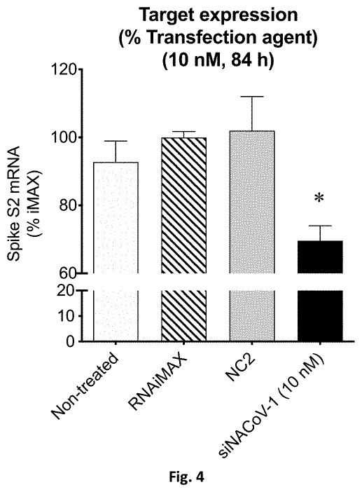

Stability of chemically modified siRNAs against SARS-CoV-2 spike (S) glycoprotein: siRNA sequences to be used in the study were thaw and incubated at 37° C. during up to 120 min with cell serum-free culture medium added with RNase I (0.25 or 0.50 Units) or with culture medium containing 5% or 10% fetal bovine serum. In contrast to non-modified (natural) siRNAs, chemically modified siRNAs against SARS-CoV-2 spike (S) glycoprotein show a significant resistance to degradation in culture medium containing 5% or 10% fetal bovine serum (FIG. 1) or RNAse I (0.50 Units) for up to 120 min (FIG. 2). These chemically modified siRNAs against SARS-CoV-2 spike (S) glycoprotein retain their capacity in RISC engagement and downregulation of SARS-CoV-2 spike (S) glycoprotein mRNA expression (FIG. 3).

### EXAMPLE 5

Cell culture: Vero 6E (VERO C1008) cells were maintained in a humidified atmosphere of 5% CO₂ at 37° C. Cells were grown in Eagles' Mimimun Essential Medium (Sigma, St. Louis, Mo.) supplemented with 1 mM sodium pyruvate and 1500 mg/L sodium bicarbonate, 10% fetal bovine serum (FBS) (Cytia HyClone, USA). The medium was changed every 2 days, and cells reached confluence 3-4 days after initial seeding. For subculturing, cells were dissociated with 0.25% trypsin-ethylenediaminetetraacetic acid (EDTA) (Sigma, St. Louis, Mo.), split 1:4 and subcultured in a 21-cm² growth area (Sarstedt, Germany).

SARS-CoV-2, Isolate USA-WA1/2020, obtained from ATCC (item NR-52281; batch number 70034262, was propagated in VERO E6 (VERO C1008) cells. Infectious virus titre calculated by end-point dilution using Reed-Muench method (https://academic.oup.com/aje/article-abstract/27/3/493/99616) in the same cells used in the assay and expressed as TCID₅₀/mL (tissue culture infectious dose 50%/millilitre).

VERO 6E cells were seeded at 1×10⁴ cells/well in 100 μL of growth medium and incubated at 37° C. in a humidified 5% CO₂ atmosphere. The next day the different siRNAs (negative control NC2, S103650325 from Qiagen (Germany) and siNACoV-2) were used to transfect cells before viral exposure. After transfection, cells were incubated for 4-6 h at 37° C. in a humidified 5% CO₂ atmosphere. The transfection mixture was then removed, and cells were further incubated overnight with culture medium. The next day cells were inoculated with 100 TCID₅₀ of SARS-CoV-2, Isolate USA-WA1/2020 in a final volume of 100 μL and incubated for 60 min at 37° C. in a humidified 5% CO₂ atmosphere. After this incubation, cell supernatant was removed, and cells washed 3 times with PBS at 37° C. Growth medium (100 μL) was then added and cells incubated for 60 h. Cells were lysed with a mixture of isopropanol, lysis buffer and beta mercaptoethanol, and stored frozen at −80° C. until RNA extraction, as described above (paragraph 119).

### EXAMPLE 6

Mouse infection studies: Pregnant Balb/c mice (18 days) were separated into four groups after delivery of their offspring. Eleven new-born mice were chosen for each group. Mice in the prevention and treatment groups were intranasally administered peptide (5 mg/kg in 2 μl of PBS) 30 min before or after intranasal challenge with a viral dose of 10² TCID₅₀ (in 2 μl DMEM). Mice in the viral control group and the normal control group were intranasally administered with 2 μl of PBS 30 min before viral challenge or without viral challenge. Mouse survival rate and body weight variations were recorded up to 2 weeks after infection. On day 5 after infection, five mice in each group were randomly selected for euthanasia to collect and assess the viral titter in mouse tissues.

The treatment with siRNA-spike (S) glycoprotein from SARS-CoV-2 leads to a decrease spike (S) glycoprotein expression for virus-cell receptor interactions during viral entry into a cell and SARS-CoV-2 viral entry into a cell, and increase the survival of SARS-CoV-2 infected mice treated by intranasal administration of siRNAs targeting certain sequences of the SARS-CoV-2 spike (S) glycoprotein gene. This decrease in spike (S) glycoprotein expression by the siRNA-spike (S) glycoprotein from SARS-CoV-2 is accompanied by increase the survival of SARS-CoV-2 infected mice treated by intranasal administration of siRNAs targeting certain sequences of the SARS-CoV-2 spike (S) glycoprotein gene.

Additional aspects of the invention will be apparent to those skilled in the art, or may be learned from the practice of the invention. The objects and advantages of the invention may be realized and attained by means of the instrumentalities and combinations particularly pointed out in the appended claims.

### REFERENCES

1. Davis M E (2009). The first targeted delivery of siRNA in humans via a self-assembling, cyclodextrin polymer-based nanoparticle: from concept to clinic. Mol Pharm 6: 659-668.
2. Elbashir S M, Harborth J, Lendeckel W, Yalcin A, Weber K, & Tuschl T (2001). Duplexes of 21-nucleotide RNAs mediate RNA interference in cultured mammalian cells. Nature 411: 494-498.
3. Fire A, Xu S, Montgomery M K, Kostas S A, Driver S E, & Mello C C (1998). Potent and specific genetic interference by double-stranded RNA in Caenorhabditis elegans. Nature 391: 806-811.
4. Guan W J, Ni Z Y, Hu Y, Liang W H, Ou C Q, He J X, et al. (2020). Clinical Characteristics of Coronavirus Disease 2019 in China. N Engl J Med.
5. Hannon G J (2002). RNA interference. Nature 418: 244-251.
6. Harborth J, Elbashir S M, Bechert K, Tuschl T, & Weber K (2001). Identification of essential genes in cultured mammalian cells using small interfering RNAs. J Cell Sci 114: 4557-4565.
7. Hoffmann M, Kleine-Weber H, Schroeder S, Kruger N, Herrler T, Erichsen S, et al. (2020). SARS-CoV-2 Cell Entry Depends on ACE2 and TMPRSS2 and Is Blocked by a Clinically Proven Protease Inhibitor. Cell.
8. Jacque J-M, Triques K, & Stevenson M (2002). Modulation of HIV-1 replication by RNA interference. Nature 418: 435-438.
9. Lee N S, Dohjima T, Bauer G, Li H, Li M-J, Ehsani A, et al. (2001). Expression of small interfering RNAs targeted against HIV-1 rev transcripts in human cells. Nature Biotechnology 19: 500-505.
10. Lee N S, Dohjima T, Bauer G, Li H, Li M-J, Ehsani A, et al. (2002). Expression of small interfering RNAs targeted against HIV-1 rev transcripts in human cells. Nature Biotechnology 20: 500-505.
11. Li G, & De Clercq E (2020). Therapeutic options for the 2019 novel coronavirus (2019-nCoV). Nat Rev Drug Discov 19: 149-150.
12. Liu K, Fang Y Y, Deng Y, Liu W, Wang M F, Ma J P, et al. (2020). Clinical characteristics of novel coronavirus cases in tertiary hospitals in Hubei Province. Chin Med J (Engl).
13. Miyagishi M, & Taira K (2002). U6 promoter-driven siRNAs with four uridine 3′ overhangs efficiently suppress targeted gene expression in mammalian cells. Nature Biotechnology 19: 497-500.
14. Paddison P J, Caudy A A, Bernstein E, Hannon G J, & Conklin D S S (2002). Short hairpin RNAs (shRNAs) induce sequence-specific silencing in mammalian cells. Genes Dev 16: 948-958.
15. Paul C P, Good P D, Winer I, & Engelke D R (2002). Effective Expression of Small Interfering RNA in human cells. Nature Biotechnology 19: 505-508.
16. Sui G, Soohoo C, Affar E B, Gay F, Shi Y, Forrester W C, et al. (2002). A DNA vector-based RNAi technology to suppress gene expression in mammalian cells. Proc Natl Acad Sci USA 99: 5515-5520.
17. Xia H, Mao Q, Paulson H L, & Davidson B L (2002). siRNA-mediated gene silencing in vitro and in vivo. Nat Biotechnol 20: 1006-1010.
18. Yu J Y, DeRuiter S L, & Turner D L (2002). RNA interference by expression of short-interfering RNAs and hairpin RNAs in mammalian cells. Proc Natl Acad Sci USA 99: 6047-6052.
19. Zhou P, Yang X L, Wang X G, Hu B, Zhang L, Zhang W, et al. (2020). A pneumonia outbreak associated with a new coronavirus of probable bat origin. Nature 579: 270-273.
20. Zumla A, Chan J F, Azhar E I, Hui D S, & Yuen K Y (2016). Coronaviruses—drug discovery and therapeutic options. Nat Rev Drug Discov 15: 327-347.

1. An isolated or synthetic siNA (short interfering nucleic acid) molecule, wherein said molecule comprises a nucleic acid sequence selected from the group consisting of: SEQ ID No 340 to SEQ ID No 678, preferably SEQ ID No 374, SEQ ID No 375, SEQ ID No 452, SEQ ID No 453, SEQ ID No 500, SEQ ID No 501, SEQ ID No 520, SEQ ID No 556, SEQ ID No 562, SEQ ID No 563, SEQ ID No 565, SEQ ID No 566, SEQ ID No 569, SEQ ID No 570, SEQ ID No 642, SEQ ID No 643, SEQ ID No 644, SEQ ID No 646, SEQ ID No 647, SEQ ID No 648, SEQ ID No 666 to SEQ ID No 668, SEQ ID No 672, SEQ ID No 676 and SEQ ID No 677, more preferably SEQ ID No 375, SEQ ID No 452, SEQ ID No 500, SEQ ID No 501, SEQ ID No 520, SEQ ID No 556, SEQ ID No 563, SEQ ID No 566, SEQ ID No 648, SEQ ID No 666, SEQ ID No 668, SEQ ID No 671, SEQ ID No 672, SEQ ID No 676, SEQ ID No 677, and variants thereof.

2. The siNA molecule of claim 1, wherein said siNA molecule is complementary to a nucleic acid sequence selected from the group consisting of: SEQ ID No 679 to SEQ ID No 1017, preferably SEQ ID No 713, SEQ ID No 714, SEQ ID No 791, SEQ ID No 792, SEQ ID No 839, SEQ ID No 840, SEQ ID No 859, SEQ ID No 895, SEQ ID No 901, SEQ ID No 902, SEQ ID No 904, SEQ ID No 905, SEQ ID No 908, SEQ ID No 909, SEQ ID No 981, SEQ ID No 982, SEQ ID No 983, SEQ ID No 985, SEQ ID No 986, SEQ ID No 987, SEQ ID No 1005 to SEQ ID No 1007, SEQ ID No 1010, SEQ ID No 1011, SEQ ID No 1015, SEQ ID No 1016, more preferably SEQ ID No SEQ ID No 714, SEQ ID No 791, SEQ ID No 839, SEQ ID No 840, SEQ ID No 859, SEQ ID No 895, SEQ ID No 902, SEQ ID No 905, SEQ ID No 987, SEQ ID No 1005, SEQ ID No 1007, SEQ ID No 1010, SEQ ID No 1011, SEQ ID No 1015, SEQ ID No 1016, and variants thereof.

3. The siNA molecule of claim 1, wherein said molecule is between 19 and 25 base pairs in length.

4. The siNA molecule of claim 1, wherein said molecule is between 21 and 23 base pairs in length.

5. The siNA molecule of claim 1, wherein said molecule comprises at least a sequence selected from SEQ ID No 340 to SEQ ID No 1017.

6. The siNA molecule of claim 1, wherein siNA is selected from dsRNA, siRNA or shRNA.

7. The siNA molecule of claim 6, wherein siNA is siRNA.

8. The siNA molecule of claim 1, wherein siNA comprises 5′ and/or 3′ overhangs.

9. The siNA molecule of claim 1, wherein siNA comprises at least one chemical modification.

10. The siNA molecule of claim 1, wherein the siNA molecule reduces the expression of the gene for spike (S) glycoprotein from SARS-CoV-2.

11. The siNA molecule of claim 1, for use in preventing and treating infectious diseases, preferably a virus infection.

12. The siNA molecule of claim 1, wherein the siRNA molecule comprises at least one sequence selected from the group consisting of: SEQ ID No 374, SEQ ID No 375, SEQ ID No 452, SEQ ID No 453, SEQ ID No 500, SEQ ID No 501, SEQ ID No 520, SEQ ID No 556, SEQ ID No 562, SEQ ID No 563, SEQ ID No 565, SEQ ID No 566, SEQ ID No 569, SEQ ID No 570, SEQ ID No 642, SEQ ID No 643, SEQ ID No 644, SEQ ID No 646, SEQ ID No 647, SEQ ID No 648, SEQ ID No 666, SEQ ID No 667, SEQ ID No 668, SEQ ID No 671, SEQ ID No 672, SEQ ID No 676, and SEQ ID No 677, preferably, said molecule reduces the expression of the gene for spike (S) glycoprotein from SARS-CoV-2.

13. The siNA molecule of claim 1 for use in preventing and treating coronavirus-inflicted infectious conditions.

14. The siNA molecule of claim 13, wherein the coronavirus-inflicted infectious conditions is selected from the following list: SARS-CoV-2, SARS-CoV and MERS-CoV, encompassing asymptomatic infection, mild upper respiratory tract illness, severe viral pneumonia and with respiratory failure.

15. A vector, liposome, microsphere, nanoparticle or capsule comprising the siNA molecule of claim 1.

16. A pharmaceutical composition comprising at least one siRNA molecule of claim 1 and a pharmaceutically acceptable carrier.

17. The composition of claim 16, further comprising a second active ingredient for the treatment of infections by the coronavirus SARS-CoV-2.

18. The composition of claim 16, further comprising an active ingredient wherein said further active ingredient is selected from the group consisting of: anti-HIV agent, anti-malarial agent, anti-tuberculosis agent, and mixtures thereof.

19. The composition of claim 16, wherein the route of administration is selected from the group consisting of: topical application, nasal application, inhalation administration, subcutaneous injection or deposition, subcutaneous infusion, intravenous injection, and intravenous infusion.


SEQUENCE LISTING (1017 sequences)

SEQ ID NO: 1 - DNA, 21 bp, SARS coronavirus 2
  Spike (S) target protein 5′ DNA sense
aaagattgctgattataatta

SEQ ID NO: 2 - DNA, 21 bp, SARS coronavirus 2
  Spike (S) target protein 5′ DNA sense
aagattgctgattataattat

SEQ ID NO: 3 - DNA, 21 bp, SARS coronavirus 2
  Spike (S) target protein 5′ DNA sense
aaggttggtggtaattataat

SEQ ID NO: 4 - DNA, 21 bp, SARS coronavirus 2
  Spike (S) target protein 5′ DNA sense
aagtatgagcagtatataaaa

SEQ ID NO: 5 - DNA, 21 bp, SARS coronavirus 2
  Spike (S) target protein 5′ DNA sense
agtgtctcctactaaattaaa

SEQ ID NO: 6 - DNA, 21 bp, SARS coronavirus 2
  Spike (S) target protein 5′ DNA sense
gattgctgattataattataa

SEQ ID NO: 7 - DNA, 21 bp, SARS coronavirus 2
  Spike (S) target protein 5′ DNA sense
attgctgattataattataaa

SEQ ID NO: 8 - DNA, 21 bp, SARS coronavirus 2
  Spike (S) target protein 5′ DNA sense
taaggttggtggtaattataa

SEQ ID NO: 9 - DNA, 21 bp, SARS coronavirus 2
  Spike (S) target protein 5′ DNA sense
aagtatgagcagtatataaaa

SEQ ID NO: 10 - DNA, 21 bp, SARS coronavirus 2
  Spike (S) target protein 5′ DNA sense
aatccttcactgtagaaaaag

SEQ ID NO: 11 - DNA, 21 bp, SARS coronavirus 2
  Spike (S) target protein 5′ DNA sense
aacaccaggaacaaatacttc

SEQ ID NO: 12 - DNA, 21 bp, SARS coronavirus 2
  Spike (S) target protein 5′ DNA sense
aaaacaccaccaattaaagat

SEQ ID NO: 13 - DNA, 21 bp, SARS coronavirus 2
  Spike (S) target protein 5′ DNA sense
aaacaccaccaattaaagatt

SEQ ID NO: 14 - DNA, 21 bp, SARS coronavirus 2
  Spike (S) target protein 5′ DNA sense
aatcttgctgctactaaaatg

SEQ ID NO: 15 - DNA, 21 bp, SARS coronavirus 2
  Spike (S) target protein 5′ DNA sense
aaggaggagttagataaatat

SEQ ID NO: 16 - DNA, 21 bp, SARS coronavirus 2
  Spike (S) target protein 5′ DNA sense
aaagtatgagcagtatataaa

SEQ ID NO: 17 - DNA, 21 bp, SARS coronavirus 2
  Spike (S) target protein 5′ DNA sense
aaaggagtcaaattacattac

SEQ ID NO: 18 - DNA, 21 bp, SARS coronavirus 2
  Spike (S) target protein 5′ DNA sense
ctgagaagtctaacataataa

SEQ ID NO: 19 - DNA, 21 bp, SARS coronavirus 2
  Spike (S) target protein 5′ DNA sense
cgctactaatgttgttattaa

SEQ ID NO: 20 - DNA, 21 bp, SARS coronavirus 2
  Spike (S) target protein 5′ DNA sense
gctactaatgttgttattaaa

SEQ ID NO: 21 - DNA, 21 bp, SARS coronavirus 2
  Spike (S) target protein 5′ DNA sense
ttctattaaaatataatgaaa

SEQ ID NO: 22 - DNA, 21 bp, SARS coronavirus 2
  Spike (S) target protein 5′ DNA sense
tctattaaaatataatgaaaa

SEQ ID NO: 23 - DNA, 21 bp, SARS coronavirus 2
  Spike (S) target protein 5′ DNA sense
gagtgtctcctactaaattaa

SEQ ID NO: 24 - DNA, 21 bp, SARS coronavirus 2
  Spike (S) target protein 5′ DNA sense
tggaaagattgctgattataa

SEQ ID NO: 25 - DNA, 21 bp, SARS coronavirus 2
  Spike (S) target protein 5′ DNA sense
tcttactgagtctaacaaaaa

SEQ ID NO: 26 - DNA, 21 bp, SARS coronavirus 2
  Spike (S) target protein 5′ DNA sense
agtgctattggcaaaattcaa

SEQ ID NO: 27 - DNA, 21 bp, SARS coronavirus 2
  Spike (S) target protein 5′ DNA sense
catatgtgactcaacaattaa

SEQ ID NO: 28 - DNA, 21 bp, SARS coronavirus 2
  Spike (S) target protein 5′ DNA sense
gtgtacttggacaatcaaaaa

SEQ ID NO: 29 - DNA, 21 bp, SARS coronavirus 2
  Spike (S) target protein 5′ DNA sense
ttcaaggaggagttagataaa

SEQ ID NO: 30 - DNA, 21 bp, SARS coronavirus 2
  Spike (S) target protein 5′ DNA sense
tcagttgtaaacattcaaaaa

SEQ ID NO: 31 - DNA, 21 bp, SARS coronavirus 2
  Spike (S) target protein 5′ DNA sense
ttgtaaacattcaaaaagaaa

SEQ ID NO: 32 - DNA, 21 bp, SARS coronavirus 2
  Spike (S) target protein 5′ DNA sense
gaaagtatgagcagtatataa

SEQ ID NO: 33 - DNA, 21 bp, SARS coronavirus 2
  Spike (S) target protein 5′ DNA sense
aaagtatgagcagtatataaa

SEQ ID NO: 34 - DNA, 25 bp, SARS coronavirus 2
  Spike (S) target protein 5′ DNA sense
aagacccagtccctacttattgtta

SEQ ID NO: 35 - DNA, 19 bp, SARS coronavirus 2
  Spike (S) target protein 5′ DNA sense
agtccctacttattgttaa

SEQ ID NO: 36 - DNA, 22 bp, SARS coronavirus 2
  Spike (S) target protein 5′ DNA sense
agtccctacttattgttaataa

SEQ ID NO: 37 - DNA, 25 bp, SARS coronavirus 2
  Spike (S) target protein 5′ DNA sense
cccagtccctacttattgttaataa

SEQ ID NO: 38 - DNA, 21 bp, SARS coronavirus 2
  Spike (S) target protein 5′ DNA sense
aataacgctactaatgttgtt

SEQ ID NO: 39 - DNA, 21 bp, SARS coronavirus 2
  Spike (S) target protein 5′ DNA sense
aacgctactaatgttgttatt

SEQ ID NO: 40 - DNA, 21 bp, SARS coronavirus 2
  Spike (S) target protein 5′ DNA sense
aaaatataatgaaaatggaac

SEQ ID NO: 41 - DNA, 21 bp, SARS coronavirus 2
  Spike (S) target protein 5′ DNA sense
aaaatggaaccattacagatg

SEQ ID NO: 42 - DNA, 21 bp, SARS coronavirus 2
  Spike (S) target protein 5′ DNA sense
aacaaagtgtacgttgaaatc

SEQ ID NO: 43 - DNA, 21 bp, SARS coronavirus 2
  Spike (S) target protein 5′ DNA sense
aagtgtacgttgaaatccttc

SEQ ID NO: 44 - DNA, 21 bp, SARS coronavirus 2
  Spike (S) target protein 5′ DNA sense
aaatccttcactgtagaaaaa

SEQ ID NO: 45 - DNA, 21 bp, SARS coronavirus 2
  Spike (S) target protein 5′ DNA sense
aaaaaggaatctatcaaactt

SEQ ID NO: 46 - DNA, 21 bp, SARS coronavirus 2
  Spike (S) target protein 5′ DNA sense
aaaaggaatctatcaaacttc

SEQ ID NO: 47 - DNA, 21 bp, SARS coronavirus 2
  Spike (S) target protein 5′ DNA sense
aaggaatctatcaaacttcta

SEQ ID NO: 48 - DNA, 21 bp, SARS coronavirus 2
  Spike (S) target protein 5′ DNA sense
aatctatcaaacttctaactt

SEQ ID NO: 49 - DNA, 21 bp, SARS coronavirus 2
  Spike (S) target protein 5′ DNA sense
aaccaacagaatctattgtta

SEQ ID NO: 50 - DNA, 21 bp, SARS coronavirus 2
  Spike (S) target protein 5′ DNA sense
aacagaatctattgttagatt

SEQ ID NO: 51 - DNA, 21 bp, SARS coronavirus 2
  Spike (S) target protein 5′ DNA sense
aactgtgttgctgattattct

SEQ ID NO: 52 - DNA, 21 bp, SARS coronavirus 2
  Spike (S) target protein 5′ DNA sense
aaccatacagagtagtagtac

SEQ ID NO: 53 - DNA, 21 bp, SARS coronavirus 2
  Spike (S) target protein 5′ DNA sense
aacatgtcaacaactcatatg

SEQ ID NO: 54 - DNA, 21 bp, SARS coronavirus 2
  Spike (S) target protein 5′ DNA sense
aaattcagttgcttactctaa

SEQ ID NO: 55 - DNA, 21 bp, SARS coronavirus 2
  Spike (S) target protein 5′ DNA sense
aattcagttgcttactctaat

SEQ ID NO: 56 - DNA, 21 bp, SARS coronavirus 2
  Spike (S) target protein 5′ DNA sense
aaatgattgctcaatacactt

SEQ ID NO: 57 - DNA, 21 bp, SARS coronavirus 2
  Spike (S) target protein 5′ DNA sense
aatgattgctcaatacacttc

SEQ ID NO: 58 - DNA, 21 bp, SARS coronavirus 2
  Spike (S) target protein 5′ DNA sense
aagtgcacttggaaaacttca

SEQ ID NO: 59 - DNA, 21 bp, SARS coronavirus 2
  Spike (S) target protein 5′ DNA sense
aagatgtggtcaaccaaaatg

SEQ ID NO: 60 - DNA, 21 bp, SARS coronavirus 2
  Spike (S) target protein 5′ DNA sense
aaacacgcttgttaaacaact

SEQ ID NO: 61 - DNA, 21 bp, SARS coronavirus 2
  Spike (S) target protein 5′ DNA sense
aacacgcttgttaaacaactt

SEQ ID NO: 62 - DNA, 21 bp, SARS coronavirus 2
  Spike (S) target protein 5′ DNA sense
aattagagctgcagaaatcag

SEQ ID NO: 63 - DNA, 21 bp, SARS coronavirus 2
  Spike (S) target protein 5′ DNA sense
aaccacaaatcattactacag

SEQ ID NO: 64 - DNA, 21 bp, SARS coronavirus 2
  Spike (S) target protein 5′ DNA sense
aatgcttcagttgtaaacatt

SEQ ID NO: 65 - DNA, 21 bp, SARS coronavirus 2
  Spike (S) target protein 5′ DNA sense
aaacattcaaaaagaaattga

SEQ ID NO: 66 - DNA, 21 bp, SARS coronavirus 2
  Spike (S) target protein 5′ DNA sense
aacattcaaaaagaaattgac

SEQ ID NO: 67 - DNA, 21 bp, SARS coronavirus 2
  Spike (S) target protein 5′ DNA sense
aaggagtcaaattacattaca

SEQ ID NO: 68 - DNA, 21 bp, SARS coronavirus 2
  Spike (S) target protein 5′ DNA sense
gtcagtgtgttaatcttacaa

SEQ ID NO: 69 - DNA, 21 bp, SARS coronavirus 2
  Spike (S) target protein 5′ DNA sense
ccactgagaagtctaacataa

SEQ ID NO: 70 - DNA, 21 bp, SARS coronavirus 2
  Spike (S) target protein 5′ DNA sense
ttattaccacaaaaacaacaa

SEQ ID NO: 71 - DNA, 21 bp, SARS coronavirus 2
  Spike (S) target protein 5′ DNA sense
tattaccacaaaaacaacaaa

SEQ ID NO: 72 - DNA, 21 bp, SARS coronavirus 2
  Spike (S) target protein 5′ DNA sense
attaccacaaaaacaacaaaa

SEQ ID NO: 73 - DNA, 21 bp, SARS coronavirus 2
  Spike (S) target protein 5′ DNA sense
taaaatataatgaaaatggaa

SEQ ID NO: 74 - DNA, 21 bp, SARS coronavirus 2
  Spike (S) target protein 5′ DNA sense
gaaatccttcactgtagaaaa

SEQ ID NO: 75 - DNA, 21 bp, SARS coronavirus 2
  Spike (S) target protein 5′ DNA sense
aaatccttcactgtagaaaaa

SEQ ID NO: 76 - DNA, 21 bp, SARS coronavirus 2
  Spike (S) target protein 5′ DNA sense
ccttcactgtagaaaaaggaa

SEQ ID NO: 77 - DNA, 21 bp, SARS coronavirus 2
  Spike (S) target protein 5′ DNA sense
gtagaaaaaggaatctatcaa

SEQ ID NO: 78 - DNA, 21 bp, SARS coronavirus 2
  Spike (S) target protein 5′ DNA sense
tagaaaaaggaatctatcaaa

SEQ ID NO: 79 - DNA, 21 bp, SARS coronavirus 2
  Spike (S) target protein 5′ DNA sense
aggaatctatcaaacttctaa

SEQ ID NO: 80 - DNA, 21 bp, SARS coronavirus 2
  Spike (S) target protein 5′ DNA sense
tgattattctgtcctatataa

SEQ ID NO: 81 - DNA, 21 bp, SARS coronavirus 2
  Spike (S) target protein 5′ DNA sense
cgttatagcttggaattctaa

SEQ ID NO: 82 - DNA, 21 bp, SARS coronavirus 2
  Spike (S) target protein 5′ DNA sense
tatagcttggaattctaacaa

SEQ ID NO: 83 - DNA, 21 bp, SARS coronavirus 2
  Spike (S) target protein 5′ DNA sense
ttctaacaatcttgattctaa

SEQ ID NO: 84 - DNA, 21 bp, SARS coronavirus 2
  Spike (S) target protein 5′ DNA sense
gttcttactgagtctaacaaa

SEQ ID NO: 85 - DNA, 21 bp, SARS coronavirus 2
  Spike (S) target protein 5′ DNA sense
ttcttactgagtctaacaaaa

SEQ ID NO: 86 - DNA, 21 bp, SARS coronavirus 2
  Spike (S) target protein 5′ DNA sense
ttggtggtgtcagtgttataa

SEQ ID NO: 87 - DNA, 21 bp, SARS coronavirus 2
  Spike (S) target protein 5′ DNA sense
accaggaacaaatacttctaa

SEQ ID NO: 88 - DNA, 21 bp, SARS coronavirus 2
  Spike (S) target protein 5′ DNA sense
aaattcagttgcttactctaa

SEQ ID NO: 89 - DNA, 21 bp, SARS coronavirus 2
  Spike (S) target protein 5′ DNA sense
ttcagttgcttactctaataa

SEQ ID NO: 90 - DNA, 21 bp, SARS coronavirus 2
  Spike (S) target protein 5′ DNA sense
agacatcagtagattgtacaa

SEQ ID NO: 91 - DNA, 21 bp, SARS coronavirus 2
  Spike (S) target protein 5′ DNA sense
tagctgttgaacaagacaaaa

SEQ ID NO: 92 - DNA, 21 bp, SARS coronavirus 2
  Spike (S) target protein 5′ DNA sense
agctgttgaacaagacaaaaa

SEQ ID NO: 93 - DNA, 21 bp, SARS coronavirus 2
  Spike (S) target protein 5′ DNA sense
ttacaaaacaccaccaattaa

SEQ ID NO: 94 - DNA, 21 bp, SARS coronavirus 2
  Spike (S) target protein 5′ DNA sense
tacaaaacaccaccaattaaa

SEQ ID NO: 95 - DNA, 21 bp, SARS coronavirus 2
  Spike (S) target protein 5′ DNA sense
gcagatgctggcttcatcaaa

SEQ ID NO: 96 - DNA, 21 bp, SARS coronavirus 2
  Spike (S) target protein 5′ DNA sense
gatgctggcttcatcaaacaa

SEQ ID NO: 97 - DNA, 21 bp, SARS coronavirus 2
  Spike (S) target protein 5′ DNA sense
gtgcaggtgctgcattacaaa

SEQ ID NO: 98 - DNA, 21 bp, SARS coronavirus 2
  Spike (S) target protein 5′ DNA sense
gttctctatgagaaccaaaaa

SEQ ID NO: 99 - DNA, 21 bp, SARS coronavirus 2
  Spike (S) target protein 5′ DNA sense
agtgcacttggaaaacttcaa

SEQ ID NO: 100 - DNA, 21 bp, SARS coronavirus 2
  Spike (S) target protein 5′ DNA sense
ttaaacacgcttgttaaacaa

SEQ ID NO: 101 - DNA, 21 bp, SARS coronavirus 2
  Spike (S) target protein 5′ DNA sense
ctaatcttgctgctactaaaa

SEQ ID NO: 102 - DNA, 21 bp, SARS coronavirus 2
  Spike (S) target protein 5′ DNA sense
gtgtgtacttggacaatcaaa

SEQ ID NO: 103 - DNA, 21 bp, SARS coronavirus 2
  Spike (S) target protein 5′ DNA sense
tgtgtacttggacaatcaaaa

SEQ ID NO: 104 - DNA, 21 bp, SARS coronavirus 2
  Spike (S) target protein 5′ DNA sense
ctggtaactgtgatgttgtaa

SEQ ID NO: 105 - DNA, 21 bp, SARS coronavirus 2
  Spike (S) target protein 5′ DNA sense
actgtgatgttgtaataggaa

SEQ ID NO: 106 - DNA, 21 bp, SARS coronavirus 2
  Spike (S) target protein 5′ DNA sense
tgttgtaataggaattgtcaa

SEQ ID NO: 107 - DNA, 21 bp, SARS coronavirus 2
  Spike (S) target protein 5′ DNA sense
attcaaggaggagttagataa

SEQ ID NO: 108 - DNA, 21 bp, SARS coronavirus 2
  Spike (S) target protein 5′ DNA sense
cattaatgcttcagttgtaaa

SEQ ID NO: 109 - DNA, 21 bp, SARS coronavirus 2
  Spike (S) target protein 5′ DNA sense
gcttcagttgtaaacattcaa

SEQ ID NO: 110 - DNA, 21 bp, SARS coronavirus 2
  Spike (S) target protein 5′ DNA sense
cttcagttgtaaacattcaaa

SEQ ID NO: 111 - DNA, 21 bp, SARS coronavirus 2
  Spike (S) target protein 5′ DNA sense
ttcagttgtaaacattcaaaa

SEQ ID NO: 112 - DNA, 21 bp, SARS coronavirus 2
  Spike (S) target protein 5′ DNA sense
gttgtaaacattcaaaaagaa

SEQ ID NO: 113 - DNA, 21 bp, SARS coronavirus 2
  Spike (S) target protein 5′ DNA sense
aagacccagtccctacttatt

SEQ ID NO: 114 - DNA, 19 bp, SARS coronavirus 2
  Spike (S) target protein 5′ DNA sense
aacctgaattagactcatt

SEQ ID NO: 115 - DNA, 21 bp, SARS coronavirus 2
  Spike (S) target protein 5′ DNA sense
aatcttacaaccagaactcaa

SEQ ID NO: 116 - DNA, 21 bp, SARS coronavirus 2
  Spike (S) target protein 5′ DNA sense
aactcaggacttgttcttacc

SEQ ID NO: 117 - DNA, 21 bp, SARS coronavirus 2
  Spike (S) target protein 5′ DNA sense
aagtctaacataataagaggc

SEQ ID NO: 118 - DNA, 21 bp, SARS coronavirus 2
  Spike (S) target protein 5′ DNA sense
aatgttgttattaaagtctgt

SEQ ID NO: 119 - DNA, 21 bp, SARS coronavirus 2
  Spike (S) target protein 5′ DNA sense
aaaaacaacaaaagttggatg

SEQ ID NO: 120 - DNA, 21 bp, SARS coronavirus 2
  Spike (S) target protein 5′ DNA sense
aacaaaagttggatggaaagt

SEQ ID NO: 121 - DNA, 21 bp, SARS coronavirus 2
  Spike (S) target protein 5′ DNA sense
aaaagttggatggaaagtgag

SEQ ID NO: 122 - DNA, 21 bp, SARS coronavirus 2
  Spike (S) target protein 5′ DNA sense
aaagttggatggaaagtgagt

SEQ ID NO: 123 - DNA, 21 bp, SARS coronavirus 2
  Spike (S) target protein 5′ DNA sense
aagttggatggaaagtgagtt

SEQ ID NO: 124 - DNA, 21 bp, SARS coronavirus 2
  Spike (S) target protein 5′ DNA sense
aataggtattaacatcactag

SEQ ID NO: 125 - DNA, 21 bp, SARS coronavirus 2
  Spike (S) target protein 5′ DNA sense
aaatataatgaaaatggaacc

SEQ ID NO: 126 - DNA, 21 bp, SARS coronavirus 2
  Spike (S) target protein 5′ DNA sense
aatgaaaatggaaccattaca

SEQ ID NO: 127 - DNA, 21 bp, SARS coronavirus 2
  Spike (S) target protein 5′ DNA sense
aaatggaaccattacagatgc

SEQ ID NO: 128 - DNA, 21 bp, SARS coronavirus 2
  Spike (S) target protein 5′ DNA sense
aaacaaagtgtacgttgaaat

SEQ ID NO: 129 - DNA, 21 bp, SARS coronavirus 2
  Spike (S) target protein 5′ DNA sense
aaagtgtacgttgaaatcctt

SEQ ID NO: 130 - DNA, 21 bp, SARS coronavirus 2
  Spike (S) target protein 5′ DNA sense
aaaggaatctatcaaacttct

SEQ ID NO: 131 - DNA, 21 bp, SARS coronavirus 2
  Spike (S) target protein 5′ DNA sense
aacaggaagagaatcagcaac

SEQ ID NO: 132 - DNA, 21 bp, SARS coronavirus 2
  Spike (S) target protein 5′ DNA sense
aagagaatcagcaactgtgtt

SEQ ID NO: 133 - DNA, 21 bp, SARS coronavirus 2
  Spike (S) target protein 5′ DNA sense
aattagaggtgatgaagtcag

SEQ ID NO: 134 - DNA, 21 bp, SARS coronavirus 2
  Spike (S) target protein 5′ DNA sense
aaactggaaagattgctgatt

SEQ ID NO: 135 - DNA, 21 bp, SARS coronavirus 2
  Spike (S) target protein 5′ DNA sense
aactggaaagattgctgatta

SEQ ID NO: 136 - DNA, 21 bp, SARS coronavirus 2
  Spike (S) target protein 5′ DNA sense
aattctaacaatcttgattct

SEQ ID NO: 137 - DNA, 21 bp, SARS coronavirus 2
  Spike (S) target protein 5′ DNA sense
aacaatcttgattctaaggtt

SEQ ID NO: 138 - DNA, 21 bp, SARS coronavirus 2
  Spike (S) target protein 5′ DNA sense
aattataattacctgtataga

SEQ ID NO: 139 - DNA, 21 bp, SARS coronavirus 2
  Spike (S) target protein 5′ DNA sense
aactcatatgagtgtgacata

SEQ ID NO: 140 - DNA, 21 bp, SARS coronavirus 2
  Spike (S) target protein 5′ DNA sense
aaaattcagttgcttactcta

SEQ ID NO: 141 - DNA, 21 bp, SARS coronavirus 2
  Spike (S) target protein 5′ DNA sense
aaattctaccagtgtctatga

SEQ ID NO: 142 - DNA, 21 bp, SARS coronavirus 2
  Spike (S) target protein 5′ DNA sense
aattctaccagtgtctatgac

SEQ ID NO: 143 - DNA, 21 bp, SARS coronavirus 2
  Spike (S) target protein 5′ DNA sense
aagacatcagtagattgtaca

SEQ ID NO: 144 - DNA, 21 bp, SARS coronavirus 2
  Spike (S) target protein 5′ DNA sense
aactggaatagctgttgaaca

SEQ ID NO: 145 - DNA, 21 bp, SARS coronavirus 2
  Spike (S) target protein 5′ DNA sense
aatagctgttgaacaagacaa

SEQ ID NO: 146 - DNA, 21 bp, SARS coronavirus 2
  Spike (S) target protein 5′ DNA sense
aaatattaccagatccatcaa

SEQ ID NO: 147 - DNA, 21 bp, SARS coronavirus 2
  Spike (S) target protein 5′ DNA sense
aatattaccagatccatcaaa

SEQ ID NO: 148 - DNA, 21 bp, SARS coronavirus 2
  Spike (S) target protein 5′ DNA sense
aacaatatggtgattgccttg

SEQ ID NO: 149 - DNA, 21 bp, SARS coronavirus 2
  Spike (S) target protein 5′ DNA sense
aatacacttctgcactgttag

SEQ ID NO: 150 - DNA, 21 bp, SARS coronavirus 2
  Spike (S) target protein 5′ DNA sense
aagtgcaaattgataggttga

SEQ ID NO: 151 - DNA, 21 bp, SARS coronavirus 2
  Spike (S) target protein 5′ DNA sense
aaattgataggttgatcacag

SEQ ID NO: 152 - DNA, 21 bp, SARS coronavirus 2
  Spike (S) target protein 5′ DNA sense
aaatcagagcttctgctaatc

SEQ ID NO: 153 - DNA, 21 bp, SARS coronavirus 2
  Spike (S) target protein 5′ DNA sense
aatcagagcttctgctaatct

SEQ ID NO: 154 - DNA, 21 bp, SARS coronavirus 2
  Spike (S) target protein 5′ DNA sense
aaaatgtcagagtgtgtactt

SEQ ID NO: 155 - DNA, 21 bp, SARS coronavirus 2
  Spike (S) target protein 5′ DNA sense
aaatgtcagagtgtgtacttg

SEQ ID NO: 156 - DNA, 21 bp, SARS coronavirus 2
  Spike (S) target protein 5′ DNA sense
aatgtcagagtgtgtacttgg

SEQ ID NO: 157 - DNA, 21 bp, SARS coronavirus 2
  Spike (S) target protein 5′ DNA sense
aagaaaagaacttcacaactg

SEQ ID NO: 158 - DNA, 21 bp, SARS coronavirus 2
  Spike (S) target protein 5′ DNA sense
aatcattactacagacaacac

SEQ ID NO: 159 - DNA, 21 bp, SARS coronavirus 2
  Spike (S) target protein 5′ DNA sense
aactgtgatgttgtaatagga

SEQ ID NO: 160 - DNA, 21 bp, SARS coronavirus 2
  Spike (S) target protein 5′ DNA sense
aataggaattgtcaacaacac

SEQ ID NO: 161 - DNA, 21 bp, SARS coronavirus 2
  Spike (S) target protein 5′ DNA sense
aacctgaattagactcattca

SEQ ID NO: 162 - DNA, 21 bp, SARS coronavirus 2
  Spike (S) target protein 5′ DNA sense
aaattgaccgcctcaatgagg

SEQ ID NO: 163 - DNA, 21 bp, SARS coronavirus 2
  Spike (S) target protein 5′ DNA sense
aatgaatctctcatcgatctc

SEQ ID NO: 164 - DNA, 21 bp, SARS coronavirus 2
  Spike (S) target protein 5′ DNA sense
aatctctcatcgatctccaag

SEQ ID NO: 165 - DNA, 21 bp, SARS coronavirus 2
  Spike (S) target protein 5′ DNA sense
aagaacttggaaagtatgagc

SEQ ID NO: 166 - DNA, 21 bp, SARS coronavirus 2
  Spike (S) target protein 5′ DNA sense
agtctctagtcagtgtgttaa

SEQ ID NO: 167 - DNA, 21 bp, SARS coronavirus 2
  Spike (S) target protein 5′ DNA sense
aatcttacaaccagaactcaa

SEQ ID NO: 168 - DNA, 21 bp, SARS coronavirus 2
  Spike (S) target protein 5′ DNA sense
tgcttccactgagaagtctaa

SEQ ID NO: 169 - DNA, 21 bp, SARS coronavirus 2
  Spike (S) target protein 5′ DNA sense
ttcttatggaccttgaaggaa

SEQ ID NO: 170 - DNA, 21 bp, SARS coronavirus 2
  Spike (S) target protein 5′ DNA sense
tcttatggaccttgaaggaaa

SEQ ID NO: 171 - DNA, 21 bp, SARS coronavirus 2
  Spike (S) target protein 5′ DNA sense
cttatggaccttgaaggaaaa

SEQ ID NO: 172 - DNA, 21 bp, SARS coronavirus 2
  Spike (S) target protein 5′ DNA sense
ttctaagcacacgcctattaa

SEQ ID NO: 173 - DNA, 21 bp, SARS coronavirus 2
  Spike (S) target protein 5′ DNA sense
agaaacaaagtgtacgttgaa

SEQ ID NO: 174 - DNA, 21 bp, SARS coronavirus 2
  Spike (S) target protein 5′ DNA sense
gaaacaaagtgtacgttgaaa

SEQ ID NO: 175 - DNA, 21 bp, SARS coronavirus 2
  Spike (S) target protein 5′ DNA sense
ttgaaatccttcactgtagaa

SEQ ID NO: 176 - DNA, 21 bp, SARS coronavirus 2
  Spike (S) target protein 5′ DNA sense
tgaaatccttcactgtagaaa

SEQ ID NO: 177 - DNA, 21 bp, SARS coronavirus 2
  Spike (S) target protein 5′ DNA sense
gaacaggaagagaatcagcaa

SEQ ID NO: 178 - DNA, 21 bp, SARS coronavirus 2
  Spike (S) target protein 5′ DNA sense
ttatggagtgtctcctactaa

SEQ ID NO: 179 - DNA, 21 bp, SARS coronavirus 2
  Spike (S) target protein 5′ DNA sense
tatggagtgtctcctactaaa

SEQ ID NO: 180 - DNA, 21 bp, SARS coronavirus 2
  Spike (S) target protein 5′ DNA sense
gaggtgatgaagtcagacaaa

SEQ ID NO: 181 - DNA, 21 bp, SARS coronavirus 2
  Spike (S) target protein 5′ DNA sense
ggccggtagcacaccttgtaa

SEQ ID NO: 182 - DNA, 21 bp, SARS coronavirus 2
  Spike (S) target protein 5′ DNA sense
tggacctaaaaagtctactaa

SEQ ID NO: 183 - DNA, 21 bp, SARS coronavirus 2
  Spike (S) target protein 5′ DNA sense
ggttaaaaacaaatgtgtcaa

SEQ ID NO: 184 - DNA, 21 bp, SARS coronavirus 2
  Spike (S) target protein 5′ DNA sense
aggtgttcttactgagtctaa

SEQ ID NO: 185 - DNA, 21 bp, SARS coronavirus 2
  Spike (S) target protein 5′ DNA sense
tgttcttactgagtctaacaa

SEQ ID NO: 186 - DNA, 21 bp, SARS coronavirus 2
  Spike (S) target protein 5′ DNA sense
tgttataacaccaggaacaaa

SEQ ID NO: 187 - DNA, 21 bp, SARS coronavirus 2
  Spike (S) target protein 5′ DNA sense
tagttatcagactcagactaa

SEQ ID NO: 188 - DNA, 21 bp, SARS coronavirus 2
  Spike (S) target protein 5′ DNA sense
tatgtcacttggtgcagaaaa

SEQ ID NO: 189 - DNA, 21 bp, SARS coronavirus 2
  Spike (S) target protein 5′ DNA sense
tctaccagtgtctatgaccaa

SEQ ID NO: 190 - DNA, 21 bp, SARS coronavirus 2
  Spike (S) target protein 5′ DNA sense
actggaatagctgttgaacaa

SEQ ID NO: 191 - DNA, 21 bp, SARS coronavirus 2
  Spike (S) target protein 5′ DNA sense
aatagctgttgaacaagacaa

SEQ ID NO: 192 - DNA, 21 bp, SARS coronavirus 2
  Spike (S) target protein 5′ DNA sense
atagctgttgaacaagacaaa

SEQ ID NO: 193 - DNA, 21 bp, SARS coronavirus 2
  Spike (S) target protein 5′ DNA sense
aaatattaccagatccatcaa

SEQ ID NO: 194 - DNA, 21 bp, SARS coronavirus 2
  Spike (S) target protein 5′ DNA sense
aatattaccagatccatcaaa

SEQ ID NO: 195 - DNA, 21 bp, SARS coronavirus 2
  Spike (S) target protein 5′ DNA sense
atattaccagatccatcaaaa

SEQ ID NO: 196 - DNA, 21 bp, SARS coronavirus 2
  Spike (S) target protein 5′ DNA sense
taccagatccatcaaaaccaa

SEQ ID NO: 197 - DNA, 21 bp, SARS coronavirus 2
  Spike (S) target protein 5′ DNA sense
tgcagatgctggcttcatcaa

SEQ ID NO: 198 - DNA, 21 bp, SARS coronavirus 2
  Spike (S) target protein 5′ DNA sense
acagatgaaatgattgctcaa

SEQ ID NO: 199 - DNA, 21 bp, SARS coronavirus 2
  Spike (S) target protein 5′ DNA sense
ggtgcaggtgctgcattacaa

SEQ ID NO: 200 - DNA, 21 bp, SARS coronavirus 2
  Spike (S) target protein 5′ DNA sense
ttaatagtgctattggcaaaa

SEQ ID NO: 201 - DNA, 21 bp, SARS coronavirus 2
  Spike (S) target protein 5′ DNA sense
cacagcaagtgcacttggaaa

SEQ ID NO: 202 - DNA, 21 bp, SARS coronavirus 2
  Spike (S) target protein 5′ DNA sense
acagcaagtgcacttggaaaa

SEQ ID NO: 203 - DNA, 21 bp, SARS coronavirus 2
  Spike (S) target protein 5′ DNA sense
ttcaagatgtggtcaaccaaa

SEQ ID NO: 204 - DNA, 21 bp, SARS coronavirus 2
  Spike (S) target protein 5′ DNA sense
tcaagatgtggtcaaccaaaa

SEQ ID NO: 205 - DNA, 21 bp, SARS coronavirus 2
  Spike (S) target protein 5′ DNA sense
gtggtcaaccaaaatgcacaa

SEQ ID NO: 206 - DNA, 21 bp, SARS coronavirus 2
  Spike (S) target protein 5′ DNA sense
aagttgaggctgaagtgcaaa

SEQ ID NO: 207 - DNA, 21 bp, SARS coronavirus 2
  Spike (S) target protein 5′ DNA sense
ttgatcacaggcagacttcaa

SEQ ID NO: 208 - DNA, 21 bp, SARS coronavirus 2
  Spike (S) target protein 5′ DNA sense
tgatcacaggcagacttcaaa

SEQ ID NO: 209 - DNA, 21 bp, SARS coronavirus 2
  Spike (S) target protein 5′ DNA sense
cagacatatgtgactcaacaa

SEQ ID NO: 210 - DNA, 21 bp, SARS coronavirus 2
  Spike (S) target protein 5′ DNA sense
agaaatcagagcttctgctaa

SEQ ID NO: 211 - DNA, 21 bp, SARS coronavirus 2
  Spike (S) target protein 5′ DNA sense
tgctaatcttgctgctactaa

SEQ ID NO: 212 - DNA, 21 bp, SARS coronavirus 2
  Spike (S) target protein 5′ DNA sense
gctaatcttgctgctactaaa

SEQ ID NO: 213 - DNA, 21 bp, SARS coronavirus 2
  Spike (S) target protein 5′ DNA sense
agtgtgtacttggacaatcaa

SEQ ID NO: 214 - DNA, 21 bp, SARS coronavirus 2
  Spike (S) target protein 5′ DNA sense
cacaagaaaagaacttcacaa

SEQ ID NO: 215 - DNA, 21 bp, SARS coronavirus 2
  Spike (S) target protein 5′ DNA sense
acaaatcattactacagacaa

SEQ ID NO: 216 - DNA, 21 bp, SARS coronavirus 2
  Spike (S) target protein 5′ DNA sense
tgtaataggaattgtcaacaa

SEQ ID NO: 217 - DNA, 21 bp, SARS coronavirus 2
  Spike (S) target protein 5′ DNA sense
acctgaattagactcattcaa

SEQ ID NO: 218 - DNA, 21 bp, SARS coronavirus 2
  Spike (S) target protein 5′ DNA sense
aggtgacatctctggcattaa

SEQ ID NO: 219 - DNA, 21 bp, SARS coronavirus 2
  Spike (S) target protein 5′ DNA sense
gcattaatgcttcagttgtaa

SEQ ID NO: 220 - DNA, 21 bp, SARS coronavirus 2
  Spike (S) target protein 5′ DNA sense
tcgatctccaagaacttggaa

SEQ ID NO: 221 - DNA, 21 bp, SARS coronavirus 2
  Spike (S) target protein 5′ DNA sense
cgatctccaagaacttggaaa

SEQ ID NO: 222 - DNA, 21 bp, SARS coronavirus 2
  Spike (S) target protein 5′ DNA sense
cagttgctgtagttgtctcaa

SEQ ID NO: 223 - DNA, 19 bp, SARS coronavirus 2
  Spike (S) target protein 5′ DNA sense
ccggtagcacaccttgtaa

SEQ ID NO: 224 - DNA, 22 bp, SARS coronavirus 2
  Spike (S) target protein 5′ DNA sense
aggccggtagcacaccttgtaa

SEQ ID NO: 225 - DNA, 25 bp, SARS coronavirus 2
  Spike (S) target protein 5′ DNA sense
atcaggccggtagcacaccttgtaa

SEQ ID NO: 226 - DNA, 19 bp, SARS coronavirus 2
  Spike (S) target protein 5′ DNA sense
ctgaattagactcattcaa

SEQ ID NO: 227 - DNA, 22 bp, SARS coronavirus 2
  Spike (S) target protein 5′ DNA sense
aacctgaattagactcattcaa

SEQ ID NO: 228 - DNA, 25 bp, SARS coronavirus 2
  Spike (S) target protein 5′ DNA sense
tgcaacctgaattagactcattcaa

SEQ ID NO: 229 - DNA, 25 bp, SARS coronavirus 2
  Spike (S) target protein 5′ DNA sense
aacctgaattagactcattcaagga

SEQ ID NO: 230 - DNA, 20 bp, SARS coronavirus 2
  Spike (S) target protein 5′ DNA sense
aaattgaccgcctcaatgag

SEQ ID NO: 231 - DNA, 19 bp, SARS coronavirus 2
  Spike (S) target protein 5′ DNA sense
aattgaccgcctcaatgag

SEQ ID NO: 232 - DNA, 21 bp, SARS coronavirus 2
  Spike (S) target protein 5′ DNA sense
aatgttacttggttccatgct

SEQ ID NO: 233 - DNA, 21 bp, SARS coronavirus 2
  Spike (S) target protein 5′ DNA sense
aaaacaacaaaagttggatgg

SEQ ID NO: 234 - DNA, 21 bp, SARS coronavirus 2
  Spike (S) target protein 5′ DNA sense
aaacaacaaaagttggatgga

SEQ ID NO: 235 - DNA, 21 bp, SARS coronavirus 2
  Spike (S) target protein 5′ DNA sense
aacaacaaaagttggatggaa

SEQ ID NO: 236 - DNA, 21 bp, SARS coronavirus 2
  Spike (S) target protein 5′ DNA sense
aaaatatattctaagcacacg

SEQ ID NO: 237 - DNA, 21 bp, SARS coronavirus 2
  Spike (S) target protein 5′ DNA sense
aatataatgaaaatggaacca

SEQ ID NO: 238 - DNA, 21 bp, SARS coronavirus 2
  Spike (S) target protein 5′ DNA sense
aatggaaccattacagatgct

SEQ ID NO: 239 - DNA, 21 bp, SARS coronavirus 2
  Spike (S) target protein 5′ DNA sense
aaccattacagatgctgtaga

SEQ ID NO: 240 - DNA, 21 bp, SARS coronavirus 2
  Spike (S) target protein 5′ DNA sense
aatcagcaactgtgttgctga

SEQ ID NO: 241 - DNA, 21 bp, SARS coronavirus 2
  Spike (S) target protein 5′ DNA sense
aattataaattaccagatgat

SEQ ID NO: 242 - DNA, 21 bp, SARS coronavirus 2
  Spike (S) target protein 5′ DNA sense
aatcttgattctaaggttggt

SEQ ID NO: 243 - DNA, 21 bp, SARS coronavirus 2
  Spike (S) target protein 5′ DNA sense
aatggtgttggttaccaacca

SEQ ID NO: 244 - DNA, 21 bp, SARS coronavirus 2
  Spike (S) target protein 5′ DNA sense
aacaaatacttctaaccaggt

SEQ ID NO: 245 - DNA, 21 bp, SARS coronavirus 2
  Spike (S) target protein 5′ DNA sense
aacaactcatatgagtgtgac

SEQ ID NO: 246 - DNA, 21 bp, SARS coronavirus 2
  Spike (S) target protein 5′ DNA sense
aatccatcattgcctacacta

SEQ ID NO: 247 - DNA, 21 bp, SARS coronavirus 2
  Spike (S) target protein 5′ DNA sense
aaaaccaagcaagaggtcatt

SEQ ID NO: 248 - DNA, 21 bp, SARS coronavirus 2
  Spike (S) target protein 5′ DNA sense
aacaaagtgacacttgcagat

SEQ ID NO: 249 - DNA, 21 bp, SARS coronavirus 2
  Spike (S) target protein 5′ DNA sense
aagtgacacttgcagatgctg

SEQ ID NO: 250 - DNA, 21 bp, SARS coronavirus 2
  Spike (S) target protein 5′ DNA sense
aaacaatatggtgattgcctt

SEQ ID NO: 251 - DNA, 21 bp, SARS coronavirus 2
  Spike (S) target protein 5′ DNA sense
aatcacttctggttggacctt

SEQ ID NO: 252 - DNA, 21 bp, SARS coronavirus 2
  Spike (S) target protein 5′ DNA sense
aatgttctctatgagaaccaa

SEQ ID NO: 253 - DNA, 21 bp, SARS coronavirus 2
  Spike (S) target protein 5′ DNA sense
aaaacttcaagatgtggtcaa

SEQ ID NO: 254 - DNA, 21 bp, SARS coronavirus 2
  Spike (S) target protein 5′ DNA sense
aaacttcaagatgtggtcaac

SEQ ID NO: 255 - DNA, 21 bp, SARS coronavirus 2
  Spike (S) target protein 5′ DNA sense
aacttcaagatgtggtcaacc

SEQ ID NO: 256 - DNA, 21 bp, SARS coronavirus 2
  Spike (S) target protein 5′ DNA sense
aaagttgaggctgaagtgcaa

SEQ ID NO: 257 - DNA, 21 bp, SARS coronavirus 2
  Spike (S) target protein 5′ DNA sense
aattgataggttgatcacagg

SEQ ID NO: 258 - DNA, 21 bp, SARS coronavirus 2
  Spike (S) target protein 5′ DNA sense
aattaattagagctgcagaaa

SEQ ID NO: 259 - DNA, 21 bp, SARS coronavirus 2
  Spike (S) target protein 5′ DNA sense
aaaagaacttcacaactgctc

SEQ ID NO: 260 - DNA, 21 bp, SARS coronavirus 2
  Spike (S) target protein 5′ DNA sense
aaagaacttcacaactgctcc

SEQ ID NO: 261 - DNA, 21 bp, SARS coronavirus 2
  Spike (S) target protein 5′ DNA sense
aaatcattactacagacaaca

SEQ ID NO: 262 - DNA, 21 bp, SARS coronavirus 2
  Spike (S) target protein 5′ DNA sense
aattagactcattcaaggagg

SEQ ID NO: 263 - DNA, 21 bp, SARS coronavirus 2
  Spike (S) target protein 5′ DNA sense
aagaatcatacatcaccagat

SEQ ID NO: 264 - DNA, 21 bp, SARS coronavirus 2
  Spike (S) target protein 5′ DNA sense
aatcatacatcaccagatgtt

SEQ ID NO: 265 - DNA, 21 bp, SARS coronavirus 2
  Spike (S) target protein 5′ DNA sense
aagaaattgaccgcctcaatg

SEQ ID NO: 266 - DNA, 21 bp, SARS coronavirus 2
  Spike (S) target protein 5′ DNA sense
aattgaccgcctcaatgaggt

SEQ ID NO: 267 - DNA, 21 bp, SARS coronavirus 2
  Spike (S) target protein 5′ DNA sense
aaatgaatctctcatcgatct

SEQ ID NO: 268 - DNA, 21 bp, SARS coronavirus 2
  Spike (S) target protein 5′ DNA sense
aacttggaaagtatgagcagt

SEQ ID NO: 269 - DNA, 21 bp, SARS coronavirus 2
  Spike (S) target protein 5′ DNA sense
gtgttaatcttacaaccagaa

SEQ ID NO: 270 - DNA, 21 bp, SARS coronavirus 2
  Spike (S) target protein 5′ DNA sense
tattgttaataacgctactaa

SEQ ID NO: 271 - DNA, 21 bp, SARS coronavirus 2
  Spike (S) target protein 5′ DNA sense
gttgttattaaagtctgtgaa

SEQ ID NO: 272 - DNA, 21 bp, SARS coronavirus 2
  Spike (S) target protein 5′ DNA sense
aacaacaaaagttggatggaa

SEQ ID NO: 273 - DNA, 21 bp, SARS coronavirus 2
  Spike (S) target protein 5′ DNA sense
acaacaaaagttggatggaaa

SEQ ID NO: 274 - DNA, 21 bp, SARS coronavirus 2
  Spike (S) target protein 5′ DNA sense
atgcttggaacaggaagagaa

SEQ ID NO: 275 - DNA, 21 bp, SARS coronavirus 2
  Spike (S) target protein 5′ DNA sense
agaggtgatgaagtcagacaa

SEQ ID NO: 276 - DNA, 21 bp, SARS coronavirus 2
  Spike (S) target protein 5′ DNA sense
aggctgcgttatagcttggaa

SEQ ID NO: 277 - DNA, 21 bp, SARS coronavirus 2
  Spike (S) target protein 5′ DNA sense
tgattctaaggttggtggtaa

SEQ ID NO: 278 - DNA, 21 bp, SARS coronavirus 2
  Spike (S) target protein 5′ DNA sense
acaccttgtaatggtgttgaa

SEQ ID NO: 279 - DNA, 21 bp, SARS coronavirus 2
  Spike (S) target protein 5′ DNA sense
actaatggtgttggttaccaa

SEQ ID NO: 280 - DNA, 21 bp, SARS coronavirus 2
  Spike (S) target protein 5′ DNA sense
gtgttataacaccaggaacaa

SEQ ID NO: 281 - DNA, 21 bp, SARS coronavirus 2
  Spike (S) target protein 5′ DNA sense
caggatgttaactgcacagaa

SEQ ID NO: 282 - DNA, 21 bp, SARS coronavirus 2
  Spike (S) target protein 5′ DNA sense
gttgctattcatgcagatcaa

SEQ ID NO: 283 - DNA, 21 bp, SARS coronavirus 2
  Spike (S) target protein 5′ DNA sense
gcacgtagtgtagctagtcaa

SEQ ID NO: 284 - DNA, 21 bp, SARS coronavirus 2
  Spike (S) target protein 5′ DNA sense
ctatgtcacttggtgcagaaa

SEQ ID NO: 285 - DNA, 21 bp, SARS coronavirus 2
  Spike (S) target protein 5′ DNA sense
actattagtgttaccacagaa

SEQ ID NO: 286 - DNA, 21 bp, SARS coronavirus 2
  Spike (S) target protein 5′ DNA sense
ctattagtgttaccacagaaa

SEQ ID NO: 287 - DNA, 21 bp, SARS coronavirus 2
  Spike (S) target protein 5′ DNA sense
tgattcaactgaatgcagcaa

SEQ ID NO: 288 - DNA, 21 bp, SARS coronavirus 2
  Spike (S) target protein 5′ DNA sense
ttaactggaatagctgttgaa

SEQ ID NO: 289 - DNA, 21 bp, SARS coronavirus 2
  Spike (S) target protein 5′ DNA sense
agatccatcaaaaccaagcaa

SEQ ID NO: 290 - DNA, 21 bp, SARS coronavirus 2
  Spike (S) target protein 5′ DNA sense
aatgttctctatgagaaccaa

SEQ ID NO: 291 - DNA, 21 bp, SARS coronavirus 2
  Spike (S) target protein 5′ DNA sense
ccacagcaagtgcacttggaa

SEQ ID NO: 292 - DNA, 21 bp, SARS coronavirus 2
  Spike (S) target protein 5′ DNA sense
aaaacttcaagatgtggtcaa

SEQ ID NO: 293 - DNA, 21 bp, SARS coronavirus 2
  Spike (S) target protein 5′ DNA sense
cttcaagatgtggtcaaccaa

SEQ ID NO: 294 - DNA, 21 bp, SARS coronavirus 2
  Spike (S) target protein 5′ DNA sense
tgttaaacaacttagctccaa

SEQ ID NO: 295 - DNA, 21 bp, SARS coronavirus 2
  Spike (S) target protein 5′ DNA sense
aaagttgaggctgaagtgcaa

SEQ ID NO: 296 - DNA, 21 bp, SARS coronavirus 2
  Spike (S) target protein 5′ DNA sense
ttgcagacatatgtgactcaa

SEQ ID NO: 297 - DNA, 21 bp, SARS coronavirus 2
  Spike (S) target protein 5′ DNA sense
aattaattagagctgcagaaa

SEQ ID NO: 298 - DNA, 21 bp, SARS coronavirus 2
  Spike (S) target protein 5′ DNA sense
tcagagtgtgtacttggacaa

SEQ ID NO: 299 - DNA, 21 bp, SARS coronavirus 2
  Spike (S) target protein 5′ DNA sense
gaatctctcatcgatctccaa

SEQ ID NO: 300 - DNA, 21 bp, SARS coronavirus 2
  Spike (S) target protein 5′ DNA sense
tctctcatcgatctccaagaa

SEQ ID NO: 301 - DNA, 21 bp, SARS coronavirus 2
  Spike (S) target protein 5′ DNA sense
tcttgtggatcctgctgcaaa

SEQ ID NO: 302 - DNA, 21 bp, SARS coronavirus 2
  Spike (S) target protein 5′ DNA sense
ccagtgctcaaaggagtcaaa

SEQ ID NO: 303 - DNA, 19 bp, SARS coronavirus 2
  Spike (S) target protein 5′ DNA sense
aagacccagtccctactta

SEQ ID NO: 304 - DNA, 19 bp, SARS coronavirus 2
  Spike (S) target protein 5′ DNA sense
ttgttaataacgctactaa

SEQ ID NO: 305 - DNA, 21 bp, SARS coronavirus 2
  Spike (S) target protein 5′ DNA sense
tattgttaataacgctactaa

SEQ ID NO: 306 - DNA, 25 bp, SARS coronavirus 2
  Spike (S) target protein 5′ DNA sense
tacttattgttaataacgctactaa

SEQ ID NO: 307 - DNA, 19 bp, SARS coronavirus 2
  Spike (S) target protein 5′ DNA sense
accttgtaatggtgttgaa

SEQ ID NO: 308 - DNA, 19 bp, SARS coronavirus 2
  Spike (S) target protein 5′ DNA sense
aactgaaatctatcaggcc

SEQ ID NO: 309 - DNA, 21 bp, SARS coronavirus 2
  Spike (S) target protein 5′ DNA sense
acaccttgtaatggtgttgaa

SEQ ID NO: 310 - DNA, 25 bp, SARS coronavirus 2
  Spike (S) target protein 5′ DNA sense
tagcacaccttgtaatggtgttgaa

SEQ ID NO: 311 - DNA, 25 bp, SARS coronavirus 2
  Spike (S) target protein 5′ DNA sense
aaatctatcaggccggtagcacacc

SEQ ID NO: 312 - DNA, 25 bp, SARS coronavirus 2
  Spike (S) target protein 5′ DNA sense
aaattgaccgcctcaatgaggttgc

SEQ ID NO: 313 - DNA, 21 bp, SARS coronavirus 2
  Spike (S) target protein 5′ DNA sense
aaatatattctaagcacacgc

SEQ ID NO: 314 - DNA, 21 bp, SARS coronavirus 2
  Spike (S) target protein 5′ DNA sense
aagtgttatggagtgtctcct

SEQ ID NO: 315 - DNA, 21 bp, SARS coronavirus 2
  Spike (S) target protein 5′ DNA sense
aacttactcctacttggcgtg

SEQ ID NO: 316 - DNA, 21 bp, SARS coronavirus 2
  Spike (S) target protein 5′ DNA sense
aaagtgacacttgcagatgct

SEQ ID NO: 317 - DNA, 21 bp, SARS coronavirus 2
  Spike (S) target protein 5′ DNA sense
aatatggtgattgccttggtg

SEQ ID NO: 318 - DNA, 21 bp, SARS coronavirus 2
  Spike (S) target protein 5′ DNA sense
aacaattaattagagctgcag

SEQ ID NO: 319 - DNA, 21 bp, SARS coronavirus 2
  Spike (S) target protein 5′ DNA sense
aagaacttcacaactgctcct

SEQ ID NO: 320 - DNA, 21 bp, SARS coronavirus 2
  Spike (S) target protein 5′ DNA sense
aaagaaattgaccgcctcaat

SEQ ID NO: 321 - DNA, 21 bp, SARS coronavirus 2
  Spike (S) target protein 5′ DNA sense
aacttctacatgcaccagcaa

SEQ ID NO: 322 - DNA, 21 bp, SARS coronavirus 2
  Spike (S) target protein 5′ DNA sense
tcagtgttataacaccaggaa

SEQ ID NO: 323 - DNA, 21 bp, SARS coronavirus 2
  Spike (S) target protein 5′ DNA sense
actatgtcacttggtgcagaa

SEQ ID NO: 324 - DNA, 21 bp, SARS coronavirus 2
  Spike (S) target protein 5′ DNA sense
cttgacaaagttgaggctgaa

SEQ ID NO: 325 - DNA, 21 bp, SARS coronavirus 2
  Spike (S) target protein 5′ DNA sense
caattaattagagctgcagaa

SEQ ID NO: 326 - DNA, 21 bp, SARS coronavirus 2
  Spike (S) target protein 5′ DNA sense
ttcttgtggatcctgctgcaa

SEQ ID NO: 327 - DNA, 21 bp, SARS coronavirus 2
  Spike (S) target protein 5′ DNA sense
aatctatcaggccggtagcac

SEQ ID NO: 328 - DNA, 22 bp, SARS coronavirus 2
  Spike (S) target protein 5′ DNA sense
aaatctatcaggccggtagcac

SEQ ID NO: 329 - DNA, 22 bp, SARS coronavirus 2
  Spike (S) target protein 5′ DNA sense
aatctatcaggccggtagcaca

SEQ ID NO: 330 - DNA, 25 bp, SARS coronavirus 2
  Spike (S) target protein 5′ DNA sense
aatctatcaggccggtagcacacct

SEQ ID NO: 331 - DNA, 25 bp, SARS coronavirus 2
  Spike (S) target protein 5′ DNA sense
aattgaccgcctcaatgaggttgcc

SEQ ID NO: 332 - DNA, 21 bp, SARS coronavirus 2
  Spike (S) target protein 5′ DNA sense
aactgaaatctatcaggccgg

SEQ ID NO: 333 - DNA, 21 bp, SARS coronavirus 2
  Spike (S) target protein 5′ DNA sense
aaatctatcaggccggtagca

SEQ ID NO: 334 - DNA, 21 bp, SARS coronavirus 2
  Spike (S) target protein 5′ DNA sense
aacttcacaactgctcctgcc

SEQ ID NO: 335 - DNA, 21 bp, SARS coronavirus 2
  Spike (S) target protein 5′ DNA sense
aaaagaaattgaccgcctcaa

SEQ ID NO: 336 - DNA, 21 bp, SARS coronavirus 2
  Spike (S) target protein 5′ DNA sense
aaaagaaattgaccgcctcaa

SEQ ID NO: 337 - DNA, 19 bp, SARS coronavirus 2
  Spike (S) target protein 5′ DNA sense
aaatctatcaggccggtag

SEQ ID NO: 338 - DNA, 19 bp, SARS coronavirus 2
  Spike (S) target protein 5′ DNA sense
aatctatcaggccggtagc

SEQ ID NO: 339 - DNA, 25 bp, SARS coronavirus 2
  Spike (S) target protein 5′ DNA sense
aactgaaatctatcaggccggtagc

SEQ ID NO: 340 - RNA, 21 nt, Artificial Sequence
  Spike (S) protein siRNA 5′ RNA sense
aaagauugcugauuauaauua

SEQ ID NO: 341 - RNA, 21 nt, Artificial Sequence
  Spike (S) protein siRNA 5′ RNA sense
aagauugcugauuauaauuau

SEQ ID NO: 342 - RNA, 21 nt, Artificial Sequence
  Spike (S) protein siRNA 5′ RNA sense
aagguuggugguaauuauaau

SEQ ID NO: 343 - RNA, 21 nt, Artificial Sequence
  Spike (S) protein siRNA 5′ RNA sense
aaguaugagcaguauauaaaa

SEQ ID NO: 344 - RNA, 21 nt, Artificial Sequence
  Spike (S) protein siRNA 5′ RNA sense
agugucuccuacuaaauuaaa

SEQ ID NO: 345 - RNA, 21 nt, Artificial Sequence
  Spike (S) protein siRNA 5′ RNA sense
gauugcugauuauaauuauaa

SEQ ID NO: 346 - RNA, 21 nt, Artificial Sequence
  Spike (S) protein siRNA 5′ RNA sense
auugcugauuauaauuauaaa

SEQ ID NO: 347 - RNA, 21 nt, Artificial Sequence
  Spike (S) protein siRNA 5′ RNA sense
uaagguuggugguaauuauaa

SEQ ID NO: 348 - RNA, 21 nt, Artificial Sequence
  Spike (S) protein siRNA 5′ RNA sense
aaguaugagcaguauauaaaa

SEQ ID NO: 349 - RNA, 21 nt, Artificial Sequence
  Spike (S) protein siRNA 5′ RNA sense
aauccuucacuguagaaaaag

SEQ ID NO: 350 - RNA, 21 nt, Artificial Sequence
  Spike (S) protein siRNA 5′ RNA sense
aacaccaggaacaaauacuuc

SEQ ID NO: 351 - RNA, 21 nt, Artificial Sequence
  Spike (S) protein siRNA 5′ RNA sense
aaaacaccaccaauuaaagau

SEQ ID NO: 352 - RNA, 21 nt, Artificial Sequence
  Spike (S) protein siRNA 5′ RNA sense
aaacaccaccaauuaaagauu

SEQ ID NO: 353 - RNA, 21 nt, Artificial Sequence
  Spike (S) protein siRNA 5′ RNA sense
aaucuugcugcuacuaaaaug

SEQ ID NO: 354 - RNA, 21 nt, Artificial Sequence
  Spike (S) protein siRNA 5′ RNA sense
aaggaggaguuagauaaauau

SEQ ID NO: 355 - RNA, 21 nt, Artificial Sequence
  Spike (S) protein siRNA 5′ RNA sense
aaaguaugagcaguauauaaa

SEQ ID NO: 356 - RNA, 21 nt, Artificial Sequence
  Spike (S) protein siRNA 5′ RNA sense
aaaggagucaaauuacauuac

SEQ ID NO: 357 - RNA, 21 nt, Artificial Sequence
  Spike (S) protein siRNA 5′ RNA sense
cugagaagucuaacauaauaa

SEQ ID NO: 358 - RNA, 21 nt, Artificial Sequence
  Spike (S) protein siRNA 5′ RNA sense
cgcuacuaauguuguuauuaa

SEQ ID NO: 359 - RNA, 21 nt, Artificial Sequence
  Spike (S) protein siRNA 5′ RNA sense
gcuacuaauguuguuauuaaa

SEQ ID NO: 360 - RNA, 21 nt, Artificial Sequence
  Spike (S) protein siRNA 5′ RNA sense
uucuauuaaaauauaaugaaa

SEQ ID NO: 361 - RNA, 21 nt, Artificial Sequence
  Spike (S) protein siRNA 5′ RNA sense
ucuauuaaaauauaaugaaaa

SEQ ID NO: 362 - RNA, 21 nt, Artificial Sequence
  Spike (S) protein siRNA 5′ RNA sense
gagugucuccuacuaaauuaa

SEQ ID NO: 363 - RNA, 21 nt, Artificial Sequence
  Spike (S) protein siRNA 5′ RNA sense
uggaaagauugcugauuauaa

SEQ ID NO: 364 - RNA, 21 nt, Artificial Sequence
  Spike (S) protein siRNA 5′ RNA sense
ucuuacugagucuaacaaaaa

SEQ ID NO: 365 - RNA, 21 nt, Artificial Sequence
  Spike (S) protein siRNA 5′ RNA sense
agugcuauuggcaaaauucaa

SEQ ID NO: 366 - RNA, 21 nt, Artificial Sequence
  Spike (S) protein siRNA 5′ RNA sense
cauaugugacucaacaauuaa

SEQ ID NO: 367 - RNA, 21 nt, Artificial Sequence
  Spike (S) protein siRNA 5′ RNA sense
guguacuuggacaaucaaaaa

SEQ ID NO: 368 - RNA, 21 nt, Artificial Sequence
  Spike (S) protein siRNA 5′ RNA sense
uucaaggaggaguuagauaaa

SEQ ID NO: 369 - RNA, 21 nt, Artificial Sequence
  Spike (S) protein siRNA 5′ RNA sense
ucaguuguaaacauucaaaaa

SEQ ID NO: 370 - RNA, 21 nt, Artificial Sequence
  Spike (S) protein siRNA 5′ RNA sense
uuguaaacauucaaaaagaaa

SEQ ID NO: 371 - RNA, 21 nt, Artificial Sequence
  Spike (S) protein siRNA 5′ RNA sense
gaaaguaugagcaguauauaa

SEQ ID NO: 372 - RNA, 21 nt, Artificial Sequence
  Spike (S) protein siRNA 5′ RNA sense
aaaguaugagcaguauauaaa

SEQ ID NO: 373 - RNA, 25 nt, Artificial Sequence
  Spike (S) protein siRNA 5′ RNA sense
aagacccagucccuacuuauuguua

SEQ ID NO: 374 - RNA, 19 nt, Artificial Sequence
  Spike (S) protein siRNA 5′ RNA sense
agucccuacuuauuguuaa

SEQ ID NO: 375 - RNA, 22 nt, Artificial Sequence
  Spike (S) protein siRNA 5′ RNA sense
agucccuacuuauuguuaauaa

SEQ ID NO: 376 - RNA, 25 nt, Artificial Sequence
  Spike (S) protein siRNA 5′ RNA sense
cccagucccuacuuauuguuaauaa

SEQ ID NO: 377 - RNA, 21 nt, Artificial Sequence
  Spike (S) protein siRNA 5′ RNA sense
aauaacgcuacuaauguuguu

SEQ ID NO: 378 - RNA, 21 nt, Artificial Sequence
  Spike (S) protein siRNA 5′ RNA sense
aacgcuacuaauguuguuauu

SEQ ID NO: 379 - RNA, 21 nt, Artificial Sequence
  Spike (S) protein siRNA 5′ RNA sense
aaaauauaaugaaaauggaac

SEQ ID NO: 380 - RNA, 21 nt, Artificial Sequence
  Spike (S) protein siRNA 5′ RNA sense
aaaauggaaccauuacagaug

SEQ ID NO: 381 - RNA, 21 nt, Artificial Sequence
  Spike (S) protein siRNA 5′ RNA sense
aacaaaguguacguugaaauc

SEQ ID NO: 382 - RNA, 21 nt, Artificial Sequence
  Spike (S) protein siRNA 5′ RNA sense
aaguguacguugaaauccuuc

SEQ ID NO: 383 - RNA, 21 nt, Artificial Sequence
  Spike (S) protein siRNA 5′ RNA sense
aaauccuucacuguagaaaaa

SEQ ID NO: 384 - RNA, 21 nt, Artificial Sequence
  Spike (S) protein siRNA 5′ RNA sense
aaaaaggaaucuaucaaacuu

SEQ ID NO: 385 - RNA, 21 nt, Artificial Sequence
  Spike (S) protein siRNA 5′ RNA sense
aaaaggaaucuaucaaacuuc

SEQ ID NO: 386 - RNA, 21 nt, Artificial Sequence
  Spike (S) protein siRNA 5′ RNA sense
aaggaaucuaucaaacuucua

SEQ ID NO: 387 - RNA, 21 nt, Artificial Sequence
  Spike (S) protein siRNA 5′ RNA sense
aaucuaucaaacuucuaacuu

SEQ ID NO: 388 - RNA, 21 nt, Artificial Sequence
  Spike (S) protein siRNA 5′ RNA sense
aaccaacagaaucuauuguua

SEQ ID NO: 389 - RNA, 21 nt, Artificial Sequence
  Spike (S) protein siRNA 5′ RNA sense
aacagaaucuauuguuagauu

SEQ ID NO: 390 - RNA, 21 nt, Artificial Sequence
  Spike (S) protein siRNA 5′ RNA sense
aacuguguugcugauuauucu

SEQ ID NO: 391 - RNA, 21 nt, Artificial Sequence
  Spike (S) protein siRNA 5′ RNA sense
aaccauacagaguaguaguac

SEQ ID NO: 392 - RNA, 21 nt, Artificial Sequence
  Spike (S) protein siRNA 5′ RNA sense
aacaugucaacaacucauaug

SEQ ID NO: 393 - RNA, 21 nt, Artificial Sequence
  Spike (S) protein siRNA 5′ RNA sense
aaauucaguugcuuacucuaa

SEQ ID NO: 394 - RNA, 21 nt, Artificial Sequence
  Spike (S) protein siRNA 5′ RNA sense
aauucaguugcuuacucuaau

SEQ ID NO: 395 - RNA, 21 nt, Artificial Sequence
  Spike (S) protein siRNA 5′ RNA sense
aaaugauugcucaauacacuu

SEQ ID NO: 396 - RNA, 21 nt, Artificial Sequence
  Spike (S) protein siRNA 5′ RNA sense
aaugauugcucaauacacuuc

SEQ ID NO: 397 - RNA, 21 nt, Artificial Sequence
  Spike (S) protein siRNA 5′ RNA sense
aagugcacuuggaaaacuuca

SEQ ID NO: 398 - RNA, 21 nt, Artificial Sequence
  Spike (S) protein siRNA 5′ RNA sense
aagauguggucaaccaaaaug

SEQ ID NO: 399 - RNA, 21 nt, Artificial Sequence
  Spike (S) protein siRNA 5′ RNA sense
aaacacgcuuguuaaacaacu

SEQ ID NO: 400 - RNA, 21 nt, Artificial Sequence
  Spike (S) protein siRNA 5′ RNA sense
aacacgcuuguuaaacaacuu

SEQ ID NO: 401 - RNA, 21 nt, Artificial Sequence
  Spike (S) protein siRNA 5′ RNA sense
aauuagagcugcagaaaucag

SEQ ID NO: 402 - RNA, 21 nt, Artificial Sequence
  Spike (S) protein siRNA 5′ RNA sense
aaccacaaaucauuacuacag

SEQ ID NO: 403 - RNA, 21 nt, Artificial Sequence
  Spike (S) protein siRNA 5′ RNA sense
aaugcuucaguuguaaacauu

SEQ ID NO: 404 - RNA, 21 nt, Artificial Sequence
  Spike (S) protein siRNA 5′ RNA sense
aaacauucaaaaagaaauuga

SEQ ID NO: 405 - RNA, 21 nt, Artificial Sequence
  Spike (S) protein siRNA 5′ RNA sense
aacauucaaaaagaaauugac

SEQ ID NO: 406 - RNA, 21 nt, Artificial Sequence
  Spike (S) protein siRNA 5′ RNA sense
aaggagucaaauuacauuaca

SEQ ID NO: 407 - RNA, 21 nt, Artificial Sequence
  Spike (S) protein siRNA 5′ RNA sense
gucaguguguuaaucuuacaa

SEQ ID NO: 408 - RNA, 21 nt, Artificial Sequence
  Spike (S) protein siRNA 5′ RNA sense
ccacugagaagucuaacauaa

SEQ ID NO: 409 - RNA, 21 nt, Artificial Sequence
  Spike (S) protein siRNA 5′ RNA sense
uuauuaccacaaaaacaacaa

SEQ ID NO: 410 - RNA, 21 nt, Artificial Sequence
  Spike (S) protein siRNA 5′ RNA sense
uauuaccacaaaaacaacaaa

SEQ ID NO: 411 - RNA, 21 nt, Artificial Sequence
  Spike (S) protein siRNA 5′ RNA sense
auuaccacaaaaacaacaaaa

SEQ ID NO: 412 - RNA, 21 nt, Artificial Sequence
  Spike (S) protein siRNA 5′ RNA sense
uaaaauauaaugaaaauggaa

SEQ ID NO: 413 - RNA, 21 nt, Artificial Sequence
  Spike (S) protein siRNA 5′ RNA sense
gaaauccuucacuguagaaaa

SEQ ID NO: 414 - RNA, 21 nt, Artificial Sequence
  Spike (S) protein siRNA 5′ RNA sense
aaauccuucacuguagaaaaa

SEQ ID NO: 415 - RNA, 21 nt, Artificial Sequence
  Spike (S) protein siRNA 5′ RNA sense
ccuucacuguagaaaaaggaa

SEQ ID NO: 416 - RNA, 21 nt, Artificial Sequence
  Spike (S) protein siRNA 5′ RNA sense
guagaaaaaggaaucuaucaa

SEQ ID NO: 417 - RNA, 21 nt, Artificial Sequence
  Spike (S) protein siRNA 5′ RNA sense
uagaaaaaggaaucuaucaaa

SEQ ID NO: 418 - RNA, 21 nt, Artificial Sequence
  Spike (S) protein siRNA 5′ RNA sense
aggaaucuaucaaacuucuaa

SEQ ID NO: 419 - RNA, 21 nt, Artificial Sequence
  Spike (S) protein siRNA 5′ RNA sense
ugauuauucuguccuauauaa

SEQ ID NO: 420 - RNA, 21 nt, Artificial Sequence
  Spike (S) protein siRNA 5′ RNA sense
cguuauagcuuggaauucuaa

SEQ ID NO: 421 - RNA, 21 nt, Artificial Sequence
  Spike (S) protein siRNA 5′ RNA sense
uauagcuuggaauucuaacaa

SEQ ID NO: 422 - RNA, 21 nt, Artificial Sequence
  Spike (S) protein siRNA 5′ RNA sense
uucuaacaaucuugauucuaa

SEQ ID NO: 423 - RNA, 21 nt, Artificial Sequence
  Spike (S) protein siRNA 5′ RNA sense
guucuuacugagucuaacaaa

SEQ ID NO: 424 - RNA, 21 nt, Artificial Sequence
  Spike (S) protein siRNA 5′ RNA sense
uucuuacugagucuaacaaaa

SEQ ID NO: 425 - RNA, 21 nt, Artificial Sequence
  Spike (S) protein siRNA 5′ RNA sense
uugguggugucaguguuauaa

SEQ ID NO: 426 - RNA, 21 nt, Artificial Sequence
  Spike (S) protein siRNA 5′ RNA sense
accaggaacaaauacuucuaa

SEQ ID NO: 427 - RNA, 21 nt, Artificial Sequence
  Spike (S) protein siRNA 5′ RNA sense
aaauucaguugcuuacucuaa

SEQ ID NO: 428 - RNA, 21 nt, Artificial Sequence
  Spike (S) protein siRNA 5′ RNA sense
uucaguugcuuacucuaauaa

SEQ ID NO: 429 - RNA, 21 nt, Artificial Sequence
  Spike (S) protein siRNA 5′ RNA sense
agacaucaguagauuguacaa

SEQ ID NO: 430 - RNA, 21 nt, Artificial Sequence
  Spike (S) protein siRNA 5′ RNA sense
uagcuguugaacaagacaaaa

SEQ ID NO: 431 - RNA, 21 nt, Artificial Sequence
  Spike (S) protein siRNA 5′ RNA sense
agcuguugaacaagacaaaaa

SEQ ID NO: 432 - RNA, 21 nt, Artificial Sequence
  Spike (S) protein siRNA 5′ RNA sense
uuacaaaacaccaccaauuaa

SEQ ID NO: 433 - RNA, 21 nt, Artificial Sequence
  Spike (S) protein siRNA 5′ RNA sense
uacaaaacaccaccaauuaaa

SEQ ID NO: 434 - RNA, 21 nt, Artificial Sequence
  Spike (S) protein siRNA 5′ RNA sense
gcagaugcuggcuucaucaaa

SEQ ID NO: 435 - RNA, 21 nt, Artificial Sequence
  Spike (S) protein siRNA 5′ RNA sense
gaugcuggcuucaucaaacaa

SEQ ID NO: 436 - RNA, 21 nt, Artificial Sequence
  Spike (S) protein siRNA 5′ RNA sense
gugcaggugcugcauuacaaa

SEQ ID NO: 437 - RNA, 21 nt, Artificial Sequence
  Spike (S) protein siRNA 5′ RNA sense
guucucuaugagaaccaaaaa

SEQ ID NO: 438 - RNA, 21 nt, Artificial Sequence
  Spike (S) protein siRNA 5′ RNA sense
agugcacuuggaaaacuucaa

SEQ ID NO: 439 - RNA, 21 nt, Artificial Sequence
  Spike (S) protein siRNA 5′ RNA sense
uuaaacacgcuuguuaaacaa

SEQ ID NO: 440 - RNA, 21 nt, Artificial Sequence
  Spike (S) protein siRNA 5′ RNA sense
cuaaucuugcugcuacuaaaa

SEQ ID NO: 441 - RNA, 21 nt, Artificial Sequence
  Spike (S) protein siRNA 5′ RNA sense
guguguacuuggacaaucaaa

SEQ ID NO: 442 - RNA, 21 nt, Artificial Sequence
  Spike (S) protein siRNA 5′ RNA sense
uguguacuuggacaaucaaaa

SEQ ID NO: 443 - RNA, 21 nt, Artificial Sequence
  Spike (S) protein siRNA 5′ RNA sense
cugguaacugugauguuguaa

SEQ ID NO: 444 - RNA, 21 nt, Artificial Sequence
  Spike (S) protein siRNA 5′ RNA sense
acugugauguuguaauaggaa

SEQ ID NO: 445 - RNA, 21 nt, Artificial Sequence
  Spike (S) protein siRNA 5′ RNA sense
uguuguaauaggaauugucaa

SEQ ID NO: 446 - RNA, 21 nt, Artificial Sequence
  Spike (S) protein siRNA 5′ RNA sense
auucaaggaggaguuagauaa

SEQ ID NO: 447 - RNA, 21 nt, Artificial Sequence
  Spike (S) protein siRNA 5′ RNA sense
cauuaaugcuucaguuguaaa

SEQ ID NO: 448 - RNA, 21 nt, Artificial Sequence
  Spike (S) protein siRNA 5′ RNA sense
gcuucaguuguaaacauucaa

SEQ ID NO: 449 - RNA, 21 nt, Artificial Sequence
  Spike (S) protein siRNA 5′ RNA sense
cuucaguuguaaacauucaaa

SEQ ID NO: 450 - RNA, 21 nt, Artificial Sequence
  Spike (S) protein siRNA 5′ RNA sense
uucaguuguaaacauucaaaa

SEQ ID NO: 451 - RNA, 21 nt, Artificial Sequence
  Spike (S) protein siRNA 5′ RNA sense
guuguaaacauucaaaaagaa

SEQ ID NO: 452 - RNA, 21 nt, Artificial Sequence
  Spike (S) protein siRNA 5′ RNA sense
aagacccagucccuacuuauu

SEQ ID NO: 453 - RNA, 19 nt, Artificial Sequence
  Spike (S) protein siRNA 5′ RNA sense
aaccugaauuagacucauu

SEQ ID NO: 454 - RNA, 21 nt, Artificial Sequence
  Spike (S) protein siRNA 5′ RNA sense
aaucuuacaaccagaacucaa

SEQ ID NO: 455 - RNA, 21 nt, Artificial Sequence
  Spike (S) protein siRNA 5′ RNA sense
aacucaggacuuguucuuacc

SEQ ID NO: 456 - RNA, 21 nt, Artificial Sequence
  Spike (S) protein siRNA 5′ RNA sense
aagucuaacauaauaagaggc

SEQ ID NO: 457 - RNA, 21 nt, Artificial Sequence
  Spike (S) protein siRNA 5′ RNA sense
aauguuguuauuaaagucugu

SEQ ID NO: 458 - RNA, 21 nt, Artificial Sequence
  Spike (S) protein siRNA 5′ RNA sense
aaaaacaacaaaaguuggaug

SEQ ID NO: 459 - RNA, 21 nt, Artificial Sequence
  Spike (S) protein siRNA 5′ RNA sense
aacaaaaguuggauggaaagu

SEQ ID NO: 460 - RNA, 21 nt, Artificial Sequence
  Spike (S) protein siRNA 5′ RNA sense
aaaaguuggauggaaagugag

SEQ ID NO: 461 - RNA, 21 nt, Artificial Sequence
  Spike (S) protein siRNA 5′ RNA sense
aaaguuggauggaaagugagu

SEQ ID NO: 462 - RNA, 21 nt, Artificial Sequence
  Spike (S) protein siRNA 5′ RNA sense
aaguuggauggaaagugaguu

SEQ ID NO: 463 - RNA, 21 nt, Artificial Sequence
  Spike (S) protein siRNA 5′ RNA sense
aauagguauuaacaucacuag

SEQ ID NO: 464 - RNA, 21 nt, Artificial Sequence
  Spike (S) protein siRNA 5′ RNA sense
aaauauaaugaaaauggaacc

SEQ ID NO: 465 - RNA, 21 nt, Artificial Sequence
  Spike (S) protein siRNA 5′ RNA sense
aaugaaaauggaaccauuaca

SEQ ID NO: 466 - RNA, 21 nt, Artificial Sequence
  Spike (S) protein siRNA 5′ RNA sense
aaauggaaccauuacagaugc

SEQ ID NO: 467 - RNA, 21 nt, Artificial Sequence
  Spike (S) protein siRNA 5′ RNA sense
aaacaaaguguacguugaaau

SEQ ID NO: 468 - RNA, 21 nt, Artificial Sequence
  Spike (S) protein siRNA 5′ RNA sense
aaaguguacguugaaauccuu

SEQ ID NO: 469 - RNA, 21 nt, Artificial Sequence
  Spike (S) protein siRNA 5′ RNA sense
aaaggaaucuaucaaacuucu

SEQ ID NO: 470 - RNA, 21 nt, Artificial Sequence
  Spike (S) protein siRNA 5′ RNA sense
aacaggaagagaaucagcaac

SEQ ID NO: 471 - RNA, 21 nt, Artificial Sequence
  Spike (S) protein siRNA 5′ RNA sense
aagagaaucagcaacuguguu

SEQ ID NO: 472 - RNA, 21 nt, Artificial Sequence
  Spike (S) protein siRNA 5′ RNA sense
aauuagaggugaugaagucag

SEQ ID NO: 473 - RNA, 21 nt, Artificial Sequence
  Spike (S) protein siRNA 5′ RNA sense
aaacuggaaagauugcugauu

SEQ ID NO: 474 - RNA, 21 nt, Artificial Sequence
  Spike (S) protein siRNA 5′ RNA sense
aacuggaaagauugcugauua

SEQ ID NO: 475 - RNA, 21 nt, Artificial Sequence
  Spike (S) protein siRNA 5′ RNA sense
aauucuaacaaucuugauucu

SEQ ID NO: 476 - RNA, 21 nt, Artificial Sequence
  Spike (S) protein siRNA 5′ RNA sense
aacaaucuugauucuaagguu

SEQ ID NO: 477 - RNA, 21 nt, Artificial Sequence
  Spike (S) protein siRNA 5′ RNA sense
aauuauaauuaccuguauaga

SEQ ID NO: 478 - RNA, 21 nt, Artificial Sequence
  Spike (S) protein siRNA 5′ RNA sense
aacucauaugagugugacaua

SEQ ID NO: 479 - RNA, 21 nt, Artificial Sequence
  Spike (S) protein siRNA 5′ RNA sense
aaaauucaguugcuuacucua

SEQ ID NO: 480 - RNA, 21 nt, Artificial Sequence
  Spike (S) protein siRNA 5′ RNA sense
aaauucuaccagugucuauga

SEQ ID NO: 481 - RNA, 21 nt, Artificial Sequence
  Spike (S) protein siRNA 5′ RNA sense
aauucuaccagugucuaugac

SEQ ID NO: 482 - RNA, 21 nt, Artificial Sequence
  Spike (S) protein siRNA 5′ RNA sense
aagacaucaguagauuguaca

SEQ ID NO: 483 - RNA, 21 nt, Artificial Sequence
  Spike (S) protein siRNA 5′ RNA sense
aacuggaauagcuguugaaca

SEQ ID NO: 484 - RNA, 21 nt, Artificial Sequence
  Spike (S) protein siRNA 5′ RNA sense
aauagcuguugaacaagacaa

SEQ ID NO: 485 - RNA, 21 nt, Artificial Sequence
  Spike (S) protein siRNA 5′ RNA sense
aaauauuaccagauccaucaa

SEQ ID NO: 486 - RNA, 21 nt, Artificial Sequence
  Spike (S) protein siRNA 5′ RNA sense
aauauuaccagauccaucaaa

SEQ ID NO: 487 - RNA, 21 nt, Artificial Sequence
  Spike (S) protein siRNA 5′ RNA sense
aacaauauggugauugccuug

SEQ ID NO: 488 - RNA, 21 nt, Artificial Sequence
  Spike (S) protein siRNA 5′ RNA sense
aauacacuucugcacuguuag

SEQ ID NO: 489 - RNA, 21 nt, Artificial Sequence
  Spike (S) protein siRNA 5′ RNA sense
aagugcaaauugauagguuga

SEQ ID NO: 490 - RNA, 21 nt, Artificial Sequence
  Spike (S) protein siRNA 5′ RNA sense
aaauugauagguugaucacag

SEQ ID NO: 491 - RNA, 21 nt, Artificial Sequence
  Spike (S) protein siRNA 5′ RNA sense
aaaucagagcuucugcuaauc

SEQ ID NO: 492 - RNA, 21 nt, Artificial Sequence
  Spike (S) protein siRNA 5′ RNA sense
aaucagagcuucugcuaaucu

SEQ ID NO: 493 - RNA, 21 nt, Artificial Sequence
  Spike (S) protein siRNA 5′ RNA sense
aaaaugucagaguguguacuu

SEQ ID NO: 494 - RNA, 21 nt, Artificial Sequence
  Spike (S) protein siRNA 5′ RNA sense
aaaugucagaguguguacuug

SEQ ID NO: 495 - RNA, 21 nt, Artificial Sequence
  Spike (S) protein siRNA 5′ RNA sense
aaugucagaguguguacuugg

SEQ ID NO: 496 - RNA, 21 nt, Artificial Sequence
  Spike (S) protein siRNA 5′ RNA sense
aagaaaagaacuucacaacug

SEQ ID NO: 497 - RNA, 21 nt, Artificial Sequence
  Spike (S) protein siRNA 5′ RNA sense
aaucauuacuacagacaacac

SEQ ID NO: 498 - RNA, 21 nt, Artificial Sequence
  Spike (S) protein siRNA 5′ RNA sense
aacugugauguuguaauagga

SEQ ID NO: 499 - RNA, 21 nt, Artificial Sequence
  Spike (S) protein siRNA 5′ RNA sense
aauaggaauugucaacaacac

SEQ ID NO: 500 - RNA, 21 nt, Artificial Sequence
  Spike (S) protein siRNA 5′ RNA sense
aaccugaauuagacucauuca

SEQ ID NO: 501 - RNA, 21 nt, Artificial Sequence
  Spike (S) protein siRNA 5′ RNA sense
aaauugaccgccucaaugagg

SEQ ID NO: 502 - RNA, 21 nt, Artificial Sequence
  Spike (S) protein siRNA 5′ RNA sense
aaugaaucucucaucgaucuc

SEQ ID NO: 503 - RNA, 21 nt, Artificial Sequence
  Spike (S) protein siRNA 5′ RNA sense
aaucucucaucgaucuccaag

SEQ ID NO: 504 - RNA, 21 nt, Artificial Sequence
  Spike (S) protein siRNA 5′ RNA sense
aagaacuuggaaaguaugagc

SEQ ID NO: 505 - RNA, 21 nt, Artificial Sequence
  Spike (S) protein siRNA 5′ RNA sense
agucucuagucaguguguuaa

SEQ ID NO: 506 - RNA, 21 nt, Artificial Sequence
  Spike (S) protein siRNA 5′ RNA sense
aaucuuacaaccagaacucaa

SEQ ID NO: 507 - RNA, 21 nt, Artificial Sequence
  Spike (S) protein siRNA 5′ RNA sense
ugcuuccacugagaagucuaa

SEQ ID NO: 508 - RNA, 21 nt, Artificial Sequence
  Spike (S) protein siRNA 5′ RNA sense
uucuuauggaccuugaaggaa

SEQ ID NO: 509 - RNA, 21 nt, Artificial Sequence
  Spike (S) protein siRNA 5′ RNA sense
ucuuauggaccuugaaggaaa

SEQ ID NO: 510 - RNA, 21 nt, Artificial Sequence
  Spike (S) protein siRNA 5′ RNA sense
cuuauggaccuugaaggaaaa

SEQ ID NO: 511 - RNA, 21 nt, Artificial Sequence
  Spike (S) protein siRNA 5′ RNA sense
uucuaagcacacgccuauuaa

SEQ ID NO: 512 - RNA, 21 nt, Artificial Sequence
  Spike (S) protein siRNA 5′ RNA sense
agaaacaaaguguacguugaa

SEQ ID NO: 513 - RNA, 21 nt, Artificial Sequence
  Spike (S) protein siRNA 5′ RNA sense
gaaacaaaguguacguugaaa

SEQ ID NO: 514 - RNA, 21 nt, Artificial Sequence
  Spike (S) protein siRNA 5′ RNA sense
uugaaauccuucacuguagaa

SEQ ID NO: 515 - RNA, 21 nt, Artificial Sequence
  Spike (S) protein siRNA 5′ RNA sense
ugaaauccuucacuguagaaa

SEQ ID NO: 516 - RNA, 21 nt, Artificial Sequence
  Spike (S) protein siRNA 5′ RNA sense
gaacaggaagagaaucagcaa

SEQ ID NO: 517 - RNA, 21 nt, Artificial Sequence
  Spike (S) protein siRNA 5′ RNA sense
uuauggagugucuccuacuaa

SEQ ID NO: 518 - RNA, 21 nt, Artificial Sequence
  Spike (S) protein siRNA 5′ RNA sense
uauggagugucuccuacuaaa

SEQ ID NO: 519 - RNA, 21 nt, Artificial Sequence
  Spike (S) protein siRNA 5′ RNA sense
gaggugaugaagucagacaaa

SEQ ID NO: 520 - RNA, 21 nt, Artificial Sequence
  Spike (S) protein siRNA 5′ RNA sense
ggccgguagcacaccuuguaa

SEQ ID NO: 521 - RNA, 21 nt, Artificial Sequence
  Spike (S) protein siRNA 5′ RNA sense
uggaccuaaaaagucuacuaa

SEQ ID NO: 522 - RNA, 21 nt, Artificial Sequence
  Spike (S) protein siRNA 5′ RNA sense
gguuaaaaacaaaugugucaa

SEQ ID NO: 523 - RNA, 21 nt, Artificial Sequence
  Spike (S) protein siRNA 5′ RNA sense
agguguucuuacugagucuaa

SEQ ID NO: 524 - RNA, 21 nt, Artificial Sequence
  Spike (S) protein siRNA 5′ RNA sense
uguucuuacugagucuaacaa

SEQ ID NO: 525 - RNA, 21 nt, Artificial Sequence
  Spike (S) protein siRNA 5′ RNA sense
uguuauaacaccaggaacaaa

SEQ ID NO: 526 - RNA, 21 nt, Artificial Sequence
  Spike (S) protein siRNA 5′ RNA sense
uaguuaucagacucagacuaa

SEQ ID NO: 527 - RNA, 21 nt, Artificial Sequence
  Spike (S) protein siRNA 5′ RNA sense
uaugucacuuggugcagaaaa

SEQ ID NO: 528 - RNA, 21 nt, Artificial Sequence
  Spike (S) protein siRNA 5′ RNA sense
ucuaccagugucuaugaccaa

SEQ ID NO: 529 - RNA, 21 nt, Artificial Sequence
  Spike (S) protein siRNA 5′ RNA sense
acuggaauagcuguugaacaa

SEQ ID NO: 530 - RNA, 21 nt, Artificial Sequence
  Spike (S) protein siRNA 5′ RNA sense
aauagcuguugaacaagacaa

SEQ ID NO: 531 - RNA, 21 nt, Artificial Sequence
  Spike (S) protein siRNA 5′ RNA sense
auagcuguugaacaagacaaa

SEQ ID NO: 532 - RNA, 21 nt, Artificial Sequence
  Spike (S) protein siRNA 5′ RNA sense
aaauauuaccagauccaucaa

SEQ ID NO: 533 - RNA, 21 nt, Artificial Sequence
  Spike (S) protein siRNA 5′ RNA sense
aauauuaccagauccaucaaa

SEQ ID NO: 534 - RNA, 21 nt, Artificial Sequence
  Spike (S) protein siRNA 5′ RNA sense
auauuaccagauccaucaaaa

SEQ ID NO: 535 - RNA, 21 nt, Artificial Sequence
  Spike (S) protein siRNA 5′ RNA sense
uaccagauccaucaaaaccaa

SEQ ID NO: 536 - RNA, 21 nt, Artificial Sequence
  Spike (S) protein siRNA 5′ RNA sense
ugcagaugcuggcuucaucaa

SEQ ID NO: 537 - RNA, 21 nt, Artificial Sequence
  Spike (S) protein siRNA 5′ RNA sense
acagaugaaaugauugcucaa

SEQ ID NO: 538 - RNA, 21 nt, Artificial Sequence
  Spike (S) protein siRNA 5′ RNA sense
ggugcaggugcugcauuacaa

SEQ ID NO: 539 - RNA, 21 nt, Artificial Sequence
  Spike (S) protein siRNA 5′ RNA sense
uuaauagugcuauuggcaaaa

SEQ ID NO: 540 - RNA, 21 nt, Artificial Sequence
  Spike (S) protein siRNA 5′ RNA sense
cacagcaagugcacuuggaaa

SEQ ID NO: 541 - RNA, 21 nt, Artificial Sequence
  Spike (S) protein siRNA 5′ RNA sense
acagcaagugcacuuggaaaa

SEQ ID NO: 542 - RNA, 21 nt, Artificial Sequence
  Spike (S) protein siRNA 5′ RNA sense
uucaagauguggucaaccaaa

SEQ ID NO: 543 - RNA, 21 nt, Artificial Sequence
  Spike (S) protein siRNA 5′ RNA sense
ucaagauguggucaaccaaaa

SEQ ID NO: 544 - RNA, 21 nt, Artificial Sequence
  Spike (S) protein siRNA 5′ RNA sense
guggucaaccaaaaugcacaa

SEQ ID NO: 545 - RNA, 21 nt, Artificial Sequence
  Spike (S) protein siRNA 5′ RNA sense
aaguugaggcugaagugcaaa

SEQ ID NO: 546 - RNA, 21 nt, Artificial Sequence
  Spike (S) protein siRNA 5′ RNA sense
uugaucacaggcagacuucaa

SEQ ID NO: 547 - RNA, 21 nt, Artificial Sequence
  Spike (S) protein siRNA 5′ RNA sense
ugaucacaggcagacuucaaa

SEQ ID NO: 548 - RNA, 21 nt, Artificial Sequence
  Spike (S) protein siRNA 5′ RNA sense
cagacauaugugacucaacaa

SEQ ID NO: 549 - RNA, 21 nt, Artificial Sequence
  Spike (S) protein siRNA 5′ RNA sense
agaaaucagagcuucugcuaa

SEQ ID NO: 550 - RNA, 21 nt, Artificial Sequence
  Spike (S) protein siRNA 5′ RNA sense
ugcuaaucuugcugcuacuaa

SEQ ID NO: 551 - RNA, 21 nt, Artificial Sequence
  Spike (S) protein siRNA 5′ RNA sense
gcuaaucuugcugcuacuaaa

SEQ ID NO: 552 - RNA, 21 nt, Artificial Sequence
  Spike (S) protein siRNA 5′ RNA sense
aguguguacuuggacaaucaa

SEQ ID NO: 553 - RNA, 21 nt, Artificial Sequence
  Spike (S) protein siRNA 5′ RNA sense
cacaagaaaagaacuucacaa

SEQ ID NO: 554 - RNA, 21 nt, Artificial Sequence
  Spike (S) protein siRNA 5′ RNA sense
acaaaucauuacuacagacaa

SEQ ID NO: 555 - RNA, 21 nt, Artificial Sequence
  Spike (S) protein siRNA 5′ RNA sense
uguaauaggaauugucaacaa

SEQ ID NO: 556 - RNA, 21 nt, Artificial Sequence
  Spike (S) protein siRNA 5′ RNA sense
accugaauuagacucauucaa

SEQ ID NO: 557 - RNA, 21 nt, Artificial Sequence
  Spike (S) protein siRNA 5′ RNA sense
aggugacaucucuggcauuaa

SEQ ID NO: 558 - RNA, 21 nt, Artificial Sequence
  Spike (S) protein siRNA 5′ RNA sense
gcauuaaugcuucaguuguaa

SEQ ID NO: 559 - RNA, 21 nt, Artificial Sequence
  Spike (S) protein siRNA 5′ RNA sense
ucgaucuccaagaacuuggaa

SEQ ID NO: 560 - RNA, 21 nt, Artificial Sequence
  Spike (S) protein siRNA 5′ RNA sense
cgaucuccaagaacuuggaaa

SEQ ID NO: 561 - RNA, 21 nt, Artificial Sequence
  Spike (S) protein siRNA 5′ RNA sense
caguugcuguaguugucucaa

SEQ ID NO: 562 - RNA, 19 nt, Artificial Sequence
  Spike (S) protein siRNA 5′ RNA sense
ccgguagcacaccuuguaa

SEQ ID NO: 563 - RNA, 22 nt, Artificial Sequence
  Spike (S) protein siRNA 5′ RNA sense
aggccgguagcacaccuuguaa

SEQ ID NO: 564 - RNA, 25 nt, Artificial Sequence
  Spike (S) protein siRNA 5′ RNA sense
aucaggccgguagcacaccuuguaa

SEQ ID NO: 565 - RNA, 19 nt, Artificial Sequence
  Spike (S) protein siRNA 5′ RNA sense
cugaauuagacucauucaa

SEQ ID NO: 566 - RNA, 22 nt, Artificial Sequence
  Spike (S) protein siRNA 5′ RNA sense
aaccugaauuagacucauucaa

SEQ ID NO: 567 - RNA, 25 nt, Artificial Sequence
  Spike (S) protein siRNA 5′ RNA sense
ugcaaccugaauuagacucauucaa

SEQ ID NO: 568 - RNA, 25 nt, Artificial Sequence
  Spike (S) protein siRNA 5′ RNA sense
aaccugaauuagacucauucaagga

SEQ ID NO: 569 - RNA, 20 nt, Artificial Sequence
  Spike (S) protein siRNA 5′ RNA sense
aaauugaccgccucaaugag

SEQ ID NO: 570 - RNA, 19 nt, Artificial Sequence
  Spike (S) protein siRNA 5′ RNA sense
aauugaccgccucaaugag

SEQ ID NO: 571 - RNA, 21 nt, Artificial Sequence
  Spike (S) protein siRNA 5′ RNA sense
aauguuacuugguuccaugcu

SEQ ID NO: 572 - RNA, 21 nt, Artificial Sequence
  Spike (S) protein siRNA 5′ RNA sense
aaaacaacaaaaguuggaugg

SEQ ID NO: 573 - RNA, 21 nt, Artificial Sequence
  Spike (S) protein siRNA 5′ RNA sense
aaacaacaaaaguuggaugga

SEQ ID NO: 574 - RNA, 21 nt, Artificial Sequence
  Spike (S) protein siRNA 5′ RNA sense
aacaacaaaaguuggauggaa

SEQ ID NO: 575 - RNA, 21 nt, Artificial Sequence
  Spike (S) protein siRNA 5′ RNA sense
aaaauauauucuaagcacacg

SEQ ID NO: 576 - RNA, 21 nt, Artificial Sequence
  Spike (S) protein siRNA 5′ RNA sense
aauauaaugaaaauggaacca

SEQ ID NO: 577 - RNA, 21 nt, Artificial Sequence
  Spike (S) protein siRNA 5′ RNA sense
aauggaaccauuacagaugcu

SEQ ID NO: 578 - RNA, 21 nt, Artificial Sequence
  Spike (S) protein siRNA 5′ RNA sense
aaccauuacagaugcuguaga

SEQ ID NO: 579 - RNA, 21 nt, Artificial Sequence
  Spike (S) protein siRNA 5′ RNA sense
aaucagcaacuguguugcuga

SEQ ID NO: 580 - RNA, 21 nt, Artificial Sequence
  Spike (S) protein siRNA 5′ RNA sense
aauuauaaauuaccagaugau

SEQ ID NO: 581 - RNA, 21 nt, Artificial Sequence
  Spike (S) protein siRNA 5′ RNA sense
aaucuugauucuaagguuggu

SEQ ID NO: 582 - RNA, 21 nt, Artificial Sequence
  Spike (S) protein siRNA 5′ RNA sense
aaugguguugguuaccaacca

SEQ ID NO: 583 - RNA, 21 nt, Artificial Sequence
  Spike (S) protein siRNA 5′ RNA sense
aacaaauacuucuaaccaggu

SEQ ID NO: 584 - RNA, 21 nt, Artificial Sequence
  Spike (S) protein siRNA 5′ RNA sense
aacaacucauaugagugugac

SEQ ID NO: 585 - RNA, 21 nt, Artificial Sequence
  Spike (S) protein siRNA 5′ RNA sense
aauccaucauugccuacacua

SEQ ID NO: 586 - RNA, 21 nt, Artificial Sequence
  Spike (S) protein siRNA 5′ RNA sense
aaaaccaagcaagaggucauu

SEQ ID NO: 587 - RNA, 21 nt, Artificial Sequence
  Spike (S) protein siRNA 5′ RNA sense
aacaaagugacacuugcagau

SEQ ID NO: 588 - RNA, 21 nt, Artificial Sequence
  Spike (S) protein siRNA 5′ RNA sense
aagugacacuugcagaugcug

SEQ ID NO: 589 - RNA, 21 nt, Artificial Sequence
  Spike (S) protein siRNA 5′ RNA sense
aaacaauauggugauugccuu

SEQ ID NO: 590 - RNA, 21 nt, Artificial Sequence
  Spike (S) protein siRNA 5′ RNA sense
aaucacuucugguuggaccuu

SEQ ID NO: 591 - RNA, 21 nt, Artificial Sequence
  Spike (S) protein siRNA 5′ RNA sense
aauguucucuaugagaaccaa

SEQ ID NO: 592 - RNA, 21 nt, Artificial Sequence
  Spike (S) protein siRNA 5′ RNA sense
aaaacuucaagauguggucaa

SEQ ID NO: 593 - RNA, 21 nt, Artificial Sequence
  Spike (S) protein siRNA 5′ RNA sense
aaacuucaagauguggucaac

SEQ ID NO: 594 - RNA, 21 nt, Artificial Sequence
  Spike (S) protein siRNA 5′ RNA sense
aacuucaagauguggucaacc

SEQ ID NO: 595 - RNA, 21 nt, Artificial Sequence
  Spike (S) protein siRNA 5′ RNA sense
aaaguugaggcugaagugcaa

SEQ ID NO: 596 - RNA, 21 nt, Artificial Sequence
  Spike (S) protein siRNA 5′ RNA sense
aauugauagguugaucacagg

SEQ ID NO: 597 - RNA, 21 nt, Artificial Sequence
  Spike (S) protein siRNA 5′ RNA sense
aauuaauuagagcugcagaaa

SEQ ID NO: 598 - RNA, 21 nt, Artificial Sequence
  Spike (S) protein siRNA 5′ RNA sense
aaaagaacuucacaacugcuc

SEQ ID NO: 599 - RNA, 21 nt, Artificial Sequence
  Spike (S) protein siRNA 5′ RNA sense
aaagaacuucacaacugcucc

SEQ ID NO: 600 - RNA, 21 nt, Artificial Sequence
  Spike (S) protein siRNA 5′ RNA sense
aaaucauuacuacagacaaca

SEQ ID NO: 601 - RNA, 21 nt, Artificial Sequence
  Spike (S) protein siRNA 5′ RNA sense
aauuagacucauucaaggagg

SEQ ID NO: 602 - RNA, 21 nt, Artificial Sequence
  Spike (S) protein siRNA 5′ RNA sense
aagaaucauacaucaccagau

SEQ ID NO: 603 - RNA, 21 nt, Artificial Sequence
  Spike (S) protein siRNA 5′ RNA sense
aaucauacaucaccagauguu

SEQ ID NO: 604 - RNA, 21 nt, Artificial Sequence
  Spike (S) protein siRNA 5′ RNA sense
aagaaauugaccgccucaaug

SEQ ID NO: 605 - RNA, 21 nt, Artificial Sequence
  Spike (S) protein siRNA 5′ RNA sense
aauugaccgccucaaugaggu

SEQ ID NO: 606 - RNA, 21 nt, Artificial Sequence
  Spike (S) protein siRNA 5′ RNA sense
aaaugaaucucucaucgaucu

SEQ ID NO: 607 - RNA, 21 nt, Artificial Sequence
  Spike (S) protein siRNA 5′ RNA sense
aacuuggaaaguaugagcagu

SEQ ID NO: 608 - RNA, 21 nt, Artificial Sequence
  Spike (S) protein siRNA 5′ RNA sense
guguuaaucuuacaaccagaa

SEQ ID NO: 609 - RNA, 21 nt, Artificial Sequence
  Spike (S) protein siRNA 5′ RNA sense
uauuguuaauaacgcuacuaa

SEQ ID NO: 610 - RNA, 21 nt, Artificial Sequence
  Spike (S) protein siRNA 5′ RNA sense
guuguuauuaaagucugugaa

SEQ ID NO: 611 - RNA, 21 nt, Artificial Sequence
  Spike (S) protein siRNA 5′ RNA sense
aacaacaaaaguuggauggaa

SEQ ID NO: 612 - RNA, 21 nt, Artificial Sequence
  Spike (S) protein siRNA 5′ RNA sense
acaacaaaaguuggauggaaa

SEQ ID NO: 613 - RNA, 21 nt, Artificial Sequence
  Spike (S) protein siRNA 5′ RNA sense
augcuuggaacaggaagagaa

SEQ ID NO: 614 - RNA, 21 nt, Artificial Sequence
  Spike (S) protein siRNA 5′ RNA sense
agaggugaugaagucagacaa

SEQ ID NO: 615 - RNA, 21 nt, Artificial Sequence
  Spike (S) protein siRNA 5′ RNA sense
aggcugcguuauagcuuggaa

SEQ ID NO: 616 - RNA, 21 nt, Artificial Sequence
  Spike (S) protein siRNA 5′ RNA sense
ugauucuaagguuggugguaa

SEQ ID NO: 617 - RNA, 21 nt, Artificial Sequence
  Spike (S) protein siRNA 5′ RNA sense
acaccuuguaaugguguugaa

SEQ ID NO: 618 - RNA, 21 nt, Artificial Sequence
  Spike (S) protein siRNA 5′ RNA sense
acuaaugguguugguuaccaa

SEQ ID NO: 619 - RNA, 21 nt, Artificial Sequence
  Spike (S) protein siRNA 5′ RNA sense
guguuauaacaccaggaacaa

SEQ ID NO: 620 - RNA, 21 nt, Artificial Sequence
  Spike (S) protein siRNA 5′ RNA sense
caggauguuaacugcacagaa

SEQ ID NO: 621 - RNA, 21 nt, Artificial Sequence
  Spike (S) protein siRNA 5′ RNA sense
guugcuauucaugcagaucaa

SEQ ID NO: 622 - RNA, 21 nt, Artificial Sequence
  Spike (S) protein siRNA 5′ RNA sense
gcacguaguguagcuagucaa

SEQ ID NO: 623 - RNA, 21 nt, Artificial Sequence
  Spike (S) protein siRNA 5′ RNA sense
cuaugucacuuggugcagaaa

SEQ ID NO: 624 - RNA, 21 nt, Artificial Sequence
  Spike (S) protein siRNA 5′ RNA sense
acuauuaguguuaccacagaa

SEQ ID NO: 625 - RNA, 21 nt, Artificial Sequence
  Spike (S) protein siRNA 5′ RNA sense
cuauuaguguuaccacagaaa

SEQ ID NO: 626 - RNA, 21 nt, Artificial Sequence
  Spike (S) protein siRNA 5′ RNA sense
ugauucaacugaaugcagcaa

SEQ ID NO: 627 - RNA, 21 nt, Artificial Sequence
  Spike (S) protein siRNA 5′ RNA sense
uuaacuggaauagcuguugaa

SEQ ID NO: 628 - RNA, 21 nt, Artificial Sequence
  Spike (S) protein siRNA 5′ RNA sense
agauccaucaaaaccaagcaa

SEQ ID NO: 629 - RNA, 21 nt, Artificial Sequence
  Spike (S) protein siRNA 5′ RNA sense
aauguucucuaugagaaccaa

SEQ ID NO: 630 - RNA, 21 nt, Artificial Sequence
  Spike (S) protein siRNA 5′ RNA sense
ccacagcaagugcacuuggaa

SEQ ID NO: 631 - RNA, 21 nt, Artificial Sequence
  Spike (S) protein siRNA 5′ RNA sense
aaaacuucaagauguggucaa

SEQ ID NO: 632 - RNA, 21 nt, Artificial Sequence
  Spike (S) protein siRNA 5′ RNA sense
cuucaagauguggucaaccaa

SEQ ID NO: 633 - RNA, 21 nt, Artificial Sequence
  Spike (S) protein siRNA 5′ RNA sense
uguuaaacaacuuagcuccaa

SEQ ID NO: 634 - RNA, 21 nt, Artificial Sequence
  Spike (S) protein siRNA 5′ RNA sense
aaaguugaggcugaagugcaa

SEQ ID NO: 635 - RNA, 21 nt, Artificial Sequence
  Spike (S) protein siRNA 5′ RNA sense
uugcagacauaugugacucaa

SEQ ID NO: 636 - RNA, 21 nt, Artificial Sequence
  Spike (S) protein siRNA 5′ RNA sense
aauuaauuagagcugcagaaa

SEQ ID NO: 637 - RNA, 21 nt, Artificial Sequence
  Spike (S) protein siRNA 5′ RNA sense
ucagaguguguacuuggacaa

SEQ ID NO: 638 - RNA, 21 nt, Artificial Sequence
  Spike (S) protein siRNA 5′ RNA sense
gaaucucucaucgaucuccaa

SEQ ID NO: 639 - RNA, 21 nt, Artificial Sequence
  Spike (S) protein siRNA 5′ RNA sense
ucucucaucgaucuccaagaa

SEQ ID NO: 640 - RNA, 21 nt, Artificial Sequence
  Spike (S) protein siRNA 5′ RNA sense
ucuuguggauccugcugcaaa

SEQ ID NO: 641 - RNA, 21 nt, Artificial Sequence
  Spike (S) protein siRNA 5′ RNA sense
ccagugcucaaaggagucaaa

SEQ ID NO: 642 - RNA, 19 nt, Artificial Sequence
  Spike (S) protein siRNA 5′ RNA sense
aagacccagucccuacuua

SEQ ID NO: 643 - RNA, 19 nt, Artificial Sequence
  Spike (S) protein siRNA 5′ RNA sense
uuguuaauaacgcuacuaa

SEQ ID NO: 644 - RNA, 21 nt, Artificial Sequence
  Spike (S) protein siRNA 5′ RNA sense
uauuguuaauaacgcuacuaa

SEQ ID NO: 645 - RNA, 25 nt, Artificial Sequence
  Spike (S) protein siRNA 5′ RNA sense
uacuuauuguuaauaacgcuacuaa

SEQ ID NO: 646 - RNA, 19 nt, Artificial Sequence
  Spike (S) protein siRNA 5′ RNA sense
accuuguaaugguguugaa

SEQ ID NO: 647 - RNA, 19 nt, Artificial Sequence
  Spike (S) protein siRNA 5′ RNA sense
aacugaaaucuaucaggcc

SEQ ID NO: 648 - RNA, 21 nt, Artificial Sequence
  Spike (S) protein siRNA 5′ RNA sense
acaccuuguaaugguguugaa

SEQ ID NO: 649 - RNA, 25 nt, Artificial Sequence
  Spike (S) protein siRNA 5′ RNA sense
uagcacaccuuguaaugguguugaa

SEQ ID NO: 650 - RNA, 25 nt, Artificial Sequence
  Spike (S) protein siRNA 5′ RNA sense
aaaucuaucaggccgguagcacacc

SEQ ID NO: 651 - RNA, 25 nt, Artificial Sequence
  Spike (S) protein siRNA 5′ RNA sense
aaauugaccgccucaaugagguugc

SEQ ID NO: 652 - RNA, 21 nt, Artificial Sequence
  Spike (S) protein siRNA 5′ RNA sense
aaauauauucuaagcacacgc

SEQ ID NO: 653 - RNA, 21 nt, Artificial Sequence
  Spike (S) protein siRNA 5′ RNA sense
aaguguuauggagugucuccu

SEQ ID NO: 654 - RNA, 21 nt, Artificial Sequence
  Spike (S) protein siRNA 5′ RNA sense
aacuuacuccuacuuggcgug

SEQ ID NO: 655 - RNA, 21 nt, Artificial Sequence
  Spike (S) protein siRNA 5′ RNA sense
aaagugacacuugcagaugcu

SEQ ID NO: 656 - RNA, 21 nt, Artificial Sequence
  Spike (S) protein siRNA 5′ RNA sense
aauauggugauugccuuggug

SEQ ID NO: 657 - RNA, 21 nt, Artificial Sequence
  Spike (S) protein siRNA 5′ RNA sense
aacaauuaauuagagcugcag

SEQ ID NO: 658 - RNA, 21 nt, Artificial Sequence
  Spike (S) protein siRNA 5′ RNA sense
aagaacuucacaacugcuccu

SEQ ID NO: 659 - RNA, 21 nt, Artificial Sequence
  Spike (S) protein siRNA 5′ RNA sense
aaagaaauugaccgccucaau

SEQ ID NO: 660 - RNA, 21 nt, Artificial Sequence
  Spike (S) protein siRNA 5′ RNA sense
aacuucuacaugcaccagcaa

SEQ ID NO: 661 - RNA, 21 nt, Artificial Sequence
  Spike (S) protein siRNA 5′ RNA sense
ucaguguuauaacaccaggaa

SEQ ID NO: 662 - RNA, 21 nt, Artificial Sequence
  Spike (S) protein siRNA 5′ RNA sense
acuaugucacuuggugcagaa

SEQ ID NO: 663 - RNA, 21 nt, Artificial Sequence
  Spike (S) protein siRNA 5′ RNA sense
cuugacaaaguugaggcugaa

SEQ ID NO: 664 - RNA, 21 nt, Artificial Sequence
  Spike (S) protein siRNA 5′ RNA sense
caauuaauuagagcugcagaa

SEQ ID NO: 665 - RNA, 21 nt, Artificial Sequence
  Spike (S) protein siRNA 5′ RNA sense
uucuuguggauccugcugcaa

SEQ ID NO: 666 - RNA, 21 nt, Artificial Sequence
  Spike (S) protein siRNA 5′ RNA sense
aaucuaucaggccgguagcac

SEQ ID NO: 667 - RNA, 22 nt, Artificial Sequence
  Spike (S) protein siRNA 5′ RNA sense
aaaucuaucaggccgguagcac

SEQ ID NO: 668 - RNA, 22 nt, Artificial Sequence
  Spike (S) protein siRNA 5′ RNA sense
aaucuaucaggccgguagcaca

SEQ ID NO: 669 - RNA, 25 nt, Artificial Sequence
  Spike (S) protein siRNA 5′ RNA sense
aaucuaucaggccgguagcacaccu

SEQ ID NO: 670 - RNA, 25 nt, Artificial Sequence
  Spike (S) protein siRNA 5′ RNA sense
aauugaccgccucaaugagguugcc

SEQ ID NO: 671 - RNA, 21 nt, Artificial Sequence
  Spike (S) protein siRNA 5′ RNA sense
aacugaaaucuaucaggccgg

SEQ ID NO: 672 - RNA, 21 nt, Artificial Sequence
  Spike (S) protein siRNA 5′ RNA sense
aaaucuaucaggccgguagca

SEQ ID NO: 673 - RNA, 21 nt, Artificial Sequence
  Spike (S) protein siRNA 5′ RNA sense
aacuucacaacugcuccugcc

SEQ ID NO: 674 - RNA, 21 nt, Artificial Sequence
  Spike (S) protein siRNA 5′ RNA sense
aaaagaaauugaccgccucaa

SEQ ID NO: 675 - RNA, 21 nt, Artificial Sequence
  Spike (S) protein siRNA 5′ RNA sense
aaaagaaauugaccgccucaa

SEQ ID NO: 676 - RNA, 19 nt, Artificial Sequence
  Spike (S) protein siRNA 5′ RNA sense
aaaucuaucaggccgguag

SEQ ID NO: 677 - RNA, 19 nt, Artificial Sequence
  Spike (S) protein siRNA 5′ RNA sense
aaucuaucaggccgguagc

SEQ ID NO: 678 - RNA, 25 nt, Artificial Sequence
  Spike (S) protein siRNA 5′ RNA sense
aacugaaaucuaucaggccgguagc

SEQ ID NO: 679 - RNA, 21 nt, Artificial Sequence
  Spike (S) protein siRNA 5′ RNA antisense
uaauuauaaucagcaaucuuu

SEQ ID NO: 680 - RNA, 21 nt, Artificial Sequence
  Spike (S) protein siRNA 5′ RNA antisense
auaauuauaaucagcaaucuu

SEQ ID NO: 681 - RNA, 21 nt, Artificial Sequence
  Spike (S) protein siRNA 5′ RNA antisense
auuauaauuaccaccaaccuu

SEQ ID NO: 682 - RNA, 21 nt, Artificial Sequence
  Spike (S) protein siRNA 5′ RNA antisense
uuuuauauacugcucauacuu

SEQ ID NO: 683 - RNA, 21 nt, Artificial Sequence
  Spike (S) protein siRNA 5′ RNA antisense
uuuaauuuaguaggagacacu

SEQ ID NO: 684 - RNA, 21 nt, Artificial Sequence
  Spike (S) protein siRNA 5′ RNA antisense
uuauaauuauaaucagcaauc

SEQ ID NO: 685 - RNA, 21 nt, Artificial Sequence
  Spike (S) protein siRNA 5′ RNA antisense
uuuauaauuauaaucagcaau

SEQ ID NO: 686 - RNA, 21 nt, Artificial Sequence
  Spike (S) protein siRNA 5′ RNA antisense
uuauaauuaccaccaaccuua

SEQ ID NO: 687 - RNA, 21 nt, Artificial Sequence
  Spike (S) protein siRNA 5′ RNA antisense
uuuuauauacugcucauacuu

SEQ ID NO: 688 - RNA, 21 nt, Artificial Sequence
  Spike (S) protein siRNA 5′ RNA antisense
cuuuuucuacagugaaggauu

SEQ ID NO: 689 - RNA, 21 nt, Artificial Sequence
  Spike (S) protein siRNA 5′ RNA antisense
gaaguauuuguuccugguguu

SEQ ID NO: 690 - RNA, 21 nt, Artificial Sequence
  Spike (S) protein siRNA 5′ RNA antisense
aucuuuaauuggugguguuuu

SEQ ID NO: 691 - RNA, 21 nt, Artificial Sequence
  Spike (S) protein siRNA 5′ RNA antisense
aaucuuuaauuggugguguuu

SEQ ID NO: 692 - RNA, 21 nt, Artificial Sequence
  Spike (S) protein siRNA 5′ RNA antisense
cauuuuaguagcagcaagauu

SEQ ID NO: 693 - RNA, 21 nt, Artificial Sequence
  Spike (S) protein siRNA 5′ RNA antisense
auauuuaucuaacuccuccuu

SEQ ID NO: 694 - RNA, 21 nt, Artificial Sequence
  Spike (S) protein siRNA 5′ RNA antisense
uuuauauacugcucauacuuu

SEQ ID NO: 695 - RNA, 21 nt, Artificial Sequence
  Spike (S) protein siRNA 5′ RNA antisense
guaauguaauuugacuccuuu

SEQ ID NO: 696 - RNA, 21 nt, Artificial Sequence
  Spike (S) protein siRNA 5′ RNA antisense
uuauuauguuagacuucucag

SEQ ID NO: 697 - RNA, 21 nt, Artificial Sequence
  Spike (S) protein siRNA 5′ RNA antisense
uuaauaacaacauuaguagcg

SEQ ID NO: 698 - RNA, 21 nt, Artificial Sequence
  Spike (S) protein siRNA 5′ RNA antisense
uuuaauaacaacauuaguagc

SEQ ID NO: 699 - RNA, 21 nt, Artificial Sequence
  Spike (S) protein siRNA 5′ RNA antisense
uuucauuauauuuuaauagaa

SEQ ID NO: 700 - RNA, 21 nt, Artificial Sequence
  Spike (S) protein siRNA 5′ RNA antisense
uuuucauuauauuuuaauaga

SEQ ID NO: 701 - RNA, 21 nt, Artificial Sequence
  Spike (S) protein siRNA 5′ RNA antisense
uuaauuuaguaggagacacuc

SEQ ID NO: 702 - RNA, 21 nt, Artificial Sequence
  Spike (S) protein siRNA 5′ RNA antisense
uuauaaucagcaaucuuucca

SEQ ID NO: 703 - RNA, 21 nt, Artificial Sequence
  Spike (S) protein siRNA 5′ RNA antisense
uuuuuguuagacucaguaaga

SEQ ID NO: 704 - RNA, 21 nt, Artificial Sequence
  Spike (S) protein siRNA 5′ RNA antisense
uugaauuuugccaauagcacu

SEQ ID NO: 705 - RNA, 21 nt, Artificial Sequence
  Spike (S) protein siRNA 5′ RNA antisense
uuaauuguugagucacauaug

SEQ ID NO: 706 - RNA, 21 nt, Artificial Sequence
  Spike (S) protein siRNA 5′ RNA antisense
uuuuugauuguccaaguacac

SEQ ID NO: 707 - RNA, 21 nt, Artificial Sequence
  Spike (S) protein siRNA 5′ RNA antisense
uuuaucuaacuccuccuugaa

SEQ ID NO: 708 - RNA, 21 nt, Artificial Sequence
  Spike (S) protein siRNA 5′ RNA antisense
uuuuugaauguuuacaacuga

SEQ ID NO: 709 - RNA, 21 nt, Artificial Sequence
  Spike (S) protein siRNA 5′ RNA antisense
uuucuuuuugaauguuuacaa

SEQ ID NO: 710 - RNA, 21 nt, Artificial Sequence
  Spike (S) protein siRNA 5′ RNA antisense
uuauauacugcucauacuuuc

SEQ ID NO: 711 - RNA, 21 nt, Artificial Sequence
  Spike (S) protein siRNA 5′ RNA antisense
uuuauauacugcucauacuuu

SEQ ID NO: 712 - RNA, 25 nt, Artificial Sequence
  Spike (S) protein siRNA 5′ RNA antisense
uaacaauaaguagggacugggucuu

SEQ ID NO: 713 - RNA, 19 nt, Artificial Sequence
  Spike (S) protein siRNA 5′ RNA antisense
uuaacaauaaguagggacu

SEQ ID NO: 714 - RNA, 22 nt, Artificial Sequence
  Spike (S) protein siRNA 5′ RNA antisense
uuauuaacaauaaguagggacu

SEQ ID NO: 715 - RNA, 25 nt, Artificial Sequence
  Spike (S) protein siRNA 5′ RNA antisense
gggucagggaugaauaacaauuauu

SEQ ID NO: 716 - RNA, 21 nt, Artificial Sequence
  Spike (S) protein siRNA 5′ RNA antisense
aacaacauuaguagcguuauu

SEQ ID NO: 717 - RNA, 21 nt, Artificial Sequence
  Spike (S) protein siRNA 5′ RNA antisense
aauaacaacauuaguagcguu

SEQ ID NO: 718 - RNA, 21 nt, Artificial Sequence
  Spike (S) protein siRNA 5′ RNA antisense
guuccauuuucauuauauuuu

SEQ ID NO: 719 - RNA, 21 nt, Artificial Sequence
  Spike (S) protein siRNA 5′ RNA antisense
caucuguaaugguuccauuuu

SEQ ID NO: 720 - RNA, 21 nt, Artificial Sequence
  Spike (S) protein siRNA 5′ RNA antisense
gauuucaacguacacuuuguu

SEQ ID NO: 721 - RNA, 21 nt, Artificial Sequence
  Spike (S) protein siRNA 5′ RNA antisense
gaaggauuucaacguacacuu

SEQ ID NO: 722 - RNA, 21 nt, Artificial Sequence
  Spike (S) protein siRNA 5′ RNA antisense
uuuuucuacagugaaggauuu

SEQ ID NO: 723 - RNA, 21 nt, Artificial Sequence
  Spike (S) protein siRNA 5′ RNA antisense
aaguuugauagauuccuuuuu

SEQ ID NO: 724 - RNA, 21 nt, Artificial Sequence
  Spike (S) protein siRNA 5′ RNA antisense
gaaguuugauagauuccuuuu

SEQ ID NO: 725 - RNA, 21 nt, Artificial Sequence
  Spike (S) protein siRNA 5′ RNA antisense
uagaaguuugauagauuccuu

SEQ ID NO: 726 - RNA, 21 nt, Artificial Sequence
  Spike (S) protein siRNA 5′ RNA antisense
aaguuagaaguuugauagauu

SEQ ID NO: 727 - RNA, 21 nt, Artificial Sequence
  Spike (S) protein siRNA 5′ RNA antisense
uaacaauagauucuguugguu

SEQ ID NO: 728 - RNA, 21 nt, Artificial Sequence
  Spike (S) protein siRNA 5′ RNA antisense
aaucuaacaauagauucuguu

SEQ ID NO: 729 - RNA, 21 nt, Artificial Sequence
  Spike (S) protein siRNA 5′ RNA antisense
agaauaaucagcaacacaguu

SEQ ID NO: 730 - RNA, 21 nt, Artificial Sequence
  Spike (S) protein siRNA 5′ RNA antisense
guacuacuacucuguaugguu

SEQ ID NO: 731 - RNA, 21 nt, Artificial Sequence
  Spike (S) protein siRNA 5′ RNA antisense
cauaugaguuguugacauguu

SEQ ID NO: 732 - RNA, 21 nt, Artificial Sequence
  Spike (S) protein siRNA 5′ RNA antisense
uuagaguaagcaacugaauuu

SEQ ID NO: 733 - RNA, 21 nt, Artificial Sequence
  Spike (S) protein siRNA 5′ RNA antisense
auuagaguaagcaacugaauu

SEQ ID NO: 734 - RNA, 21 nt, Artificial Sequence
  Spike (S) protein siRNA 5′ RNA antisense
aaguguauugagcaaucauuu

SEQ ID NO: 735 - RNA, 21 nt, Artificial Sequence
  Spike (S) protein siRNA 5′ RNA antisense
gaaguguauugagcaaucauu

SEQ ID NO: 736 - RNA, 21 nt, Artificial Sequence
  Spike (S) protein siRNA 5′ RNA antisense
ugaaguuuuccaagugcacuu

SEQ ID NO: 737 - RNA, 21 nt, Artificial Sequence
  Spike (S) protein siRNA 5′ RNA antisense
cauuuugguugaccacaucuu

SEQ ID NO: 738 - RNA, 21 nt, Artificial Sequence
  Spike (S) protein siRNA 5′ RNA antisense
aguuguuuaacaagcguguuu

SEQ ID NO: 739 - RNA, 21 nt, Artificial Sequence
  Spike (S) protein siRNA 5′ RNA antisense
aaguuguuuaacaagcguguu

SEQ ID NO: 740 - RNA, 21 nt, Artificial Sequence
  Spike (S) protein siRNA 5′ RNA antisense
cugauuucugcagcucuaauu

SEQ ID NO: 741 - RNA, 21 nt, Artificial Sequence
  Spike (S) protein siRNA 5′ RNA antisense
cuguaguaaugauuugugguu

SEQ ID NO: 742 - RNA, 21 nt, Artificial Sequence
  Spike (S) protein siRNA 5′ RNA antisense
aauguuuacaacugaagcauu

SEQ ID NO: 743 - RNA, 21 nt, Artificial Sequence
  Spike (S) protein siRNA 5′ RNA antisense
ucaauuucuuuuugaauguuu

SEQ ID NO: 744 - RNA, 21 nt, Artificial Sequence
  Spike (S) protein siRNA 5′ RNA antisense
gucaauuucuuuuugaauguu

SEQ ID NO: 745 - RNA, 21 nt, Artificial Sequence
  Spike (S) protein siRNA 5′ RNA antisense
uguaauguaauuugacuccuu

SEQ ID NO: 746 - RNA, 21 nt, Artificial Sequence
  Spike (S) protein siRNA 5′ RNA antisense
uuguaagauuaacacacugac

SEQ ID NO: 747 - RNA, 21 nt, Artificial Sequence
  Spike (S) protein siRNA 5′ RNA antisense
uuauguuagacuucucagugg

SEQ ID NO: 748 - RNA, 21 nt, Artificial Sequence
  Spike (S) protein siRNA 5′ RNA antisense
uuguuguuuuugugguaauaa

SEQ ID NO: 749 - RNA, 21 nt, Artificial Sequence
  Spike (S) protein siRNA 5′ RNA antisense
uuuguuguuuuugugguaaua

SEQ ID NO: 750 - RNA, 21 nt, Artificial Sequence
  Spike (S) protein siRNA 5′ RNA antisense
uuuuguuguuuuugugguaau

SEQ ID NO: 751 - RNA, 21 nt, Artificial Sequence
  Spike (S) protein siRNA 5′ RNA antisense
uuccauuuucauuauauuuua

SEQ ID NO: 752 - RNA, 21 nt, Artificial Sequence
  Spike (S) protein siRNA 5′ RNA antisense
uuuucuacagugaaggauuuc

SEQ ID NO: 753 - RNA, 21 nt, Artificial Sequence
  Spike (S) protein siRNA 5′ RNA antisense
uuuuucuacagugaaggauuu

SEQ ID NO: 754 - RNA, 21 nt, Artificial Sequence
  Spike (S) protein siRNA 5′ RNA antisense
uuccuuuuucuacagugaagg

SEQ ID NO: 755 - RNA, 21 nt, Artificial Sequence
  Spike (S) protein siRNA 5′ RNA antisense
uugauagauuccuuuuucuac

SEQ ID NO: 756 - RNA, 21 nt, Artificial Sequence
  Spike (S) protein siRNA 5′ RNA antisense
uuugauagauuccuuuuucua

SEQ ID NO: 757 - RNA, 21 nt, Artificial Sequence
  Spike (S) protein siRNA 5′ RNA antisense
uuagaaguuugauagauuccu

SEQ ID NO: 758 - RNA, 21 nt, Artificial Sequence
  Spike (S) protein siRNA 5′ RNA antisense
uuauauaggacagaauaauca

SEQ ID NO: 759 - RNA, 21 nt, Artificial Sequence
  Spike (S) protein siRNA 5′ RNA antisense
uuagaauuccaagcuauaacg

SEQ ID NO: 760 - RNA, 21 nt, Artificial Sequence
  Spike (S) protein siRNA 5′ RNA antisense
uuguuagaauuccaagcuaua

SEQ ID NO: 761 - RNA, 21 nt, Artificial Sequence
  Spike (S) protein siRNA 5′ RNA antisense
uuagaaucaagauuguuagaa

SEQ ID NO: 762 - RNA, 21 nt, Artificial Sequence
  Spike (S) protein siRNA 5′ RNA antisense
uuuguuagacucaguaagaac

SEQ ID NO: 763 - RNA, 21 nt, Artificial Sequence
  Spike (S) protein siRNA 5′ RNA antisense
uuuuguuagacucaguaagaa

SEQ ID NO: 764 - RNA, 21 nt, Artificial Sequence
  Spike (S) protein siRNA 5′ RNA antisense
uuauaacacugacaccaccaa

SEQ ID NO: 765 - RNA, 21 nt, Artificial Sequence
  Spike (S) protein siRNA 5′ RNA antisense
uuagaaguauuuguuccuggu

SEQ ID NO: 766 - RNA, 21 nt, Artificial Sequence
  Spike (S) protein siRNA 5′ RNA antisense
uuagaguaagcaacugaauuu

SEQ ID NO: 767 - RNA, 21 nt, Artificial Sequence
  Spike (S) protein siRNA 5′ RNA antisense
uuauuagaguaagcaacugaa

SEQ ID NO: 768 - RNA, 21 nt, Artificial Sequence
  Spike (S) protein siRNA 5′ RNA antisense
uuguacaaucuacugaugucu

SEQ ID NO: 769 - RNA, 21 nt, Artificial Sequence
  Spike (S) protein siRNA 5′ RNA antisense
uuuugucuuguucaacagcua

SEQ ID NO: 770 - RNA, 21 nt, Artificial Sequence
  Spike (S) protein siRNA 5′ RNA antisense
uuuuugucuuguucaacagcu

SEQ ID NO: 771 - RNA, 21 nt, Artificial Sequence
  Spike (S) protein siRNA 5′ RNA antisense
uuaauuggugguguuuuguaa

SEQ ID NO: 772 - RNA, 21 nt, Artificial Sequence
  Spike (S) protein siRNA 5′ RNA antisense
uuuaauuggugguguuuugua

SEQ ID NO: 773 - RNA, 21 nt, Artificial Sequence
  Spike (S) protein siRNA 5′ RNA antisense
uuugaugaagccagcaucugc

SEQ ID NO: 774 - RNA, 21 nt, Artificial Sequence
  Spike (S) protein siRNA 5′ RNA antisense
uuguuugaugaagccagcauc

SEQ ID NO: 775 - RNA, 21 nt, Artificial Sequence
  Spike (S) protein siRNA 5′ RNA antisense
uuuguaaugcagcaccugcac

SEQ ID NO: 776 - RNA, 21 nt, Artificial Sequence
  Spike (S) protein siRNA 5′ RNA antisense
uuuuugguucucauagagaac

SEQ ID NO: 777 - RNA, 21 nt, Artificial Sequence
  Spike (S) protein siRNA 5′ RNA antisense
uugaaguuuuccaagugcacu

SEQ ID NO: 778 - RNA, 21 nt, Artificial Sequence
  Spike (S) protein siRNA 5′ RNA antisense
uuguuuaacaagcguguuuaa

SEQ ID NO: 779 - RNA, 21 nt, Artificial Sequence
  Spike (S) protein siRNA 5′ RNA antisense
uuuuaguagcagcaagauuag

SEQ ID NO: 780 - RNA, 21 nt, Artificial Sequence
  Spike (S) protein siRNA 5′ RNA antisense
uuugauuguccaaguacacac

SEQ ID NO: 781 - RNA, 21 nt, Artificial Sequence
  Spike (S) protein siRNA 5′ RNA antisense
uuuugauuguccaaguacaca

SEQ ID NO: 782 - RNA, 21 nt, Artificial Sequence
  Spike (S) protein siRNA 5′ RNA antisense
uuacaacaucacaguuaccag

SEQ ID NO: 783 - RNA, 21 nt, Artificial Sequence
  Spike (S) protein siRNA 5′ RNA antisense
uuccuauuacaacaucacagu

SEQ ID NO: 784 - RNA, 21 nt, Artificial Sequence
  Spike (S) protein siRNA 5′ RNA antisense
uugacaauuccuauuacaaca

SEQ ID NO: 785 - RNA, 21 nt, Artificial Sequence
  Spike (S) protein siRNA 5′ RNA antisense
uuaucuaacuccuccuugaau

SEQ ID NO: 786 - RNA, 21 nt, Artificial Sequence
  Spike (S) protein siRNA 5′ RNA antisense
uuuacaacugaagcauuaaug

SEQ ID NO: 787 - RNA, 21 nt, Artificial Sequence
  Spike (S) protein siRNA 5′ RNA antisense
uugaauguuuacaacugaagc

SEQ ID NO: 788 - RNA, 21 nt, Artificial Sequence
  Spike (S) protein siRNA 5′ RNA antisense
uuugaauguuuacaacugaag

SEQ ID NO: 789 - RNA, 21 nt, Artificial Sequence
  Spike (S) protein siRNA 5′ RNA antisense
uuuugaauguuuacaacugaa

SEQ ID NO: 790 - RNA, 21 nt, Artificial Sequence
  Spike (S) protein siRNA 5′ RNA antisense
uucuuuuugaauguuuacaac

SEQ ID NO: 791 - RNA, 21 nt, Artificial Sequence
  Spike (S) protein siRNA 5′ RNA antisense
aauaaguagggacugggucuu

SEQ ID NO: 792 - RNA, 19 nt, Artificial Sequence
  Spike (S) protein siRNA 5′ RNA antisense
aaugagucuaauucagguu

SEQ ID NO: 793 - RNA, 21 nt, Artificial Sequence
  Spike (S) protein siRNA 5′ RNA antisense
uugaguucugguuguaagauu

SEQ ID NO: 794 - RNA, 21 nt, Artificial Sequence
  Spike (S) protein siRNA 5′ RNA antisense
gguaagaacaaguccugaguu

SEQ ID NO: 795 - RNA, 21 nt, Artificial Sequence
  Spike (S) protein siRNA 5′ RNA antisense
gccucuuauuauguuagacuu

SEQ ID NO: 796 - RNA, 21 nt, Artificial Sequence
  Spike (S) protein siRNA 5′ RNA antisense
acagacuuuaauaacaacauu

SEQ ID NO: 797 - RNA, 21 nt, Artificial Sequence
  Spike (S) protein siRNA 5′ RNA antisense
cauccaacuuuuguuguuuuu

SEQ ID NO: 798 - RNA, 21 nt, Artificial Sequence
  Spike (S) protein siRNA 5′ RNA antisense
acuuuccauccaacuuuuguu

SEQ ID NO: 799 - RNA, 21 nt, Artificial Sequence
  Spike (S) protein siRNA 5′ RNA antisense
cucacuuuccauccaacuuuu

SEQ ID NO: 800 - RNA, 21 nt, Artificial Sequence
  Spike (S) protein siRNA 5′ RNA antisense
acucacuuuccauccaacuuu

SEQ ID NO: 801 - RNA, 21 nt, Artificial Sequence
  Spike (S) protein siRNA 5′ RNA antisense
aacucacuuuccauccaacuu

SEQ ID NO: 802 - RNA, 21 nt, Artificial Sequence
  Spike (S) protein siRNA 5′ RNA antisense
cuagugauguuaauaccuauu

SEQ ID NO: 803 - RNA, 21 nt, Artificial Sequence
  Spike (S) protein siRNA 5′ RNA antisense
gguuccauuuucauuauauuu

SEQ ID NO: 804 - RNA, 21 nt, Artificial Sequence
  Spike (S) protein siRNA 5′ RNA antisense
uguaaugguuccauuuucauu

SEQ ID NO: 805 - RNA, 21 nt, Artificial Sequence
  Spike (S) protein siRNA 5′ RNA antisense
gcaucuguaaugguuccauuu

SEQ ID NO: 806 - RNA, 21 nt, Artificial Sequence
  Spike (S) protein siRNA 5′ RNA antisense
auuucaacguacacuuuguuu

SEQ ID NO: 807 - RNA, 21 nt, Artificial Sequence
  Spike (S) protein siRNA 5′ RNA antisense
aaggauuucaacguacacuuu

SEQ ID NO: 808 - RNA, 21 nt, Artificial Sequence
  Spike (S) protein siRNA 5′ RNA antisense
agaaguuugauagauuccuuu

SEQ ID NO: 809 - RNA, 21 nt, Artificial Sequence
  Spike (S) protein siRNA 5′ RNA antisense
guugcugauucucuuccuguu

SEQ ID NO: 810 - RNA, 21 nt, Artificial Sequence
  Spike (S) protein siRNA 5′ RNA antisense
aacacaguugcugauucucuu

SEQ ID NO: 811 - RNA, 21 nt, Artificial Sequence
  Spike (S) protein siRNA 5′ RNA antisense
cugacuucaucaccucuaauu

SEQ ID NO: 812 - RNA, 21 nt, Artificial Sequence
  Spike (S) protein siRNA 5′ RNA antisense
aaucagcaaucuuuccaguuu

SEQ ID NO: 813 - RNA, 21 nt, Artificial Sequence
  Spike (S) protein siRNA 5′ RNA antisense
uaaucagcaaucuuuccaguu

SEQ ID NO: 814 - RNA, 21 nt, Artificial Sequence
  Spike (S) protein siRNA 5′ RNA antisense
agaaucaagauuguuagaauu

SEQ ID NO: 815 - RNA, 21 nt, Artificial Sequence
  Spike (S) protein siRNA 5′ RNA antisense
aaccuuagaaucaagauuguu

SEQ ID NO: 816 - RNA, 21 nt, Artificial Sequence
  Spike (S) protein siRNA 5′ RNA antisense
ucuauacagguaauuauaauu

SEQ ID NO: 817 - RNA, 21 nt, Artificial Sequence
  Spike (S) protein siRNA 5′ RNA antisense
uaugucacacucauaugaguu

SEQ ID NO: 818 - RNA, 21 nt, Artificial Sequence
  Spike (S) protein siRNA 5′ RNA antisense
uagaguaagcaacugaauuuu

SEQ ID NO: 819 - RNA, 21 nt, Artificial Sequence
  Spike (S) protein siRNA 5′ RNA antisense
ucauagacacugguagaauuu

SEQ ID NO: 820 - RNA, 21 nt, Artificial Sequence
  Spike (S) protein siRNA 5′ RNA antisense
gucauagacacugguagaauu

SEQ ID NO: 821 - RNA, 21 nt, Artificial Sequence
  Spike (S) protein siRNA 5′ RNA antisense
uguacaaucuacugaugucuu

SEQ ID NO: 822 - RNA, 21 nt, Artificial Sequence
  Spike (S) protein siRNA 5′ RNA antisense
uguucaacagcuauuccaguu

SEQ ID NO: 823 - RNA, 21 nt, Artificial Sequence
  Spike (S) protein siRNA 5′ RNA antisense
uugucuuguucaacagcuauu

SEQ ID NO: 824 - RNA, 21 nt, Artificial Sequence
  Spike (S) protein siRNA 5′ RNA antisense
uugauggaucugguaauauuu

SEQ ID NO: 825 - RNA, 21 nt, Artificial Sequence
  Spike (S) protein siRNA 5′ RNA antisense
uuugauggaucugguaauauu

SEQ ID NO: 826 - RNA, 21 nt, Artificial Sequence
  Spike (S) protein siRNA 5′ RNA antisense
caaggcaaucaccauauuguu

SEQ ID NO: 827 - RNA, 21 nt, Artificial Sequence
  Spike (S) protein siRNA 5′ RNA antisense
cuaacagugcagaaguguauu

SEQ ID NO: 828 - RNA, 21 nt, Artificial Sequence
  Spike (S) protein siRNA 5′ RNA antisense
ucaaccuaucaauuugcacuu

SEQ ID NO: 829 - RNA, 21 nt, Artificial Sequence
  Spike (S) protein siRNA 5′ RNA antisense
cugugaucaaccuaucaauuu

SEQ ID NO: 830 - RNA, 21 nt, Artificial Sequence
  Spike (S) protein siRNA 5′ RNA antisense
gauuagcagaagcucugauuu

SEQ ID NO: 831 - RNA, 21 nt, Artificial Sequence
  Spike (S) protein siRNA 5′ RNA antisense
agauuagcagaagcucugauu

SEQ ID NO: 832 - RNA, 21 nt, Artificial Sequence
  Spike (S) protein siRNA 5′ RNA antisense
aaguacacacucugacauuuu

SEQ ID NO: 833 - RNA, 21 nt, Artificial Sequence
  Spike (S) protein siRNA 5′ RNA antisense
caaguacacacucugacauuu

SEQ ID NO: 834 - RNA, 21 nt, Artificial Sequence
  Spike (S) protein siRNA 5′ RNA antisense
ccaaguacacacucugacauu

SEQ ID NO: 835 - RNA, 21 nt, Artificial Sequence
  Spike (S) protein siRNA 5′ RNA antisense
caguugugaaguucuuuucuu

SEQ ID NO: 836 - RNA, 21 nt, Artificial Sequence
  Spike (S) protein siRNA 5′ RNA antisense
guguugucuguaguaaugauu

SEQ ID NO: 837 - RNA, 21 nt, Artificial Sequence
  Spike (S) protein siRNA 5′ RNA antisense
uccuauuacaacaucacaguu

SEQ ID NO: 838 - RNA, 21 nt, Artificial Sequence
  Spike (S) protein siRNA 5′ RNA antisense
guguuguugacaauuccuauu

SEQ ID NO: 839 - RNA, 21 nt, Artificial Sequence
  Spike (S) protein siRNA 5′ RNA antisense
ugaaugagucuaauucagguu

SEQ ID NO: 840 - RNA, 21 nt, Artificial Sequence
  Spike (S) protein siRNA 5′ RNA antisense
ccucauugaggcggucaauuu

SEQ ID NO: 841 - RNA, 21 nt, Artificial Sequence
  Spike (S) protein siRNA 5′ RNA antisense
gagaucgaugagagauucauu

SEQ ID NO: 842 - RNA, 21 nt, Artificial Sequence
  Spike (S) protein siRNA 5′ RNA antisense
cuuggagaucgaugagagauu

SEQ ID NO: 843 - RNA, 21 nt, Artificial Sequence
  Spike (S) protein siRNA 5′ RNA antisense
gcucauacuuuccaaguucuu

SEQ ID NO: 844 - RNA, 21 nt, Artificial Sequence
  Spike (S) protein siRNA 5′ RNA antisense
uuaacacacugacuagagacu

SEQ ID NO: 845 - RNA, 21 nt, Artificial Sequence
  Spike (S) protein siRNA 5′ RNA antisense
uugaguucugguuguaagauu

SEQ ID NO: 846 - RNA, 21 nt, Artificial Sequence
  Spike (S) protein siRNA 5′ RNA antisense
uuagacuucucaguggaagca

SEQ ID NO: 847 - RNA, 21 nt, Artificial Sequence
  Spike (S) protein siRNA 5′ RNA antisense
uuccuucaagguccauaagaa

SEQ ID NO: 848 - RNA, 21 nt, Artificial Sequence
  Spike (S) protein siRNA 5′ RNA antisense
uuuccuucaagguccauaaga

SEQ ID NO: 849 - RNA, 21 nt, Artificial Sequence
  Spike (S) protein siRNA 5′ RNA antisense
uuuuccuucaagguccauaag

SEQ ID NO: 850 - RNA, 21 nt, Artificial Sequence
  Spike (S) protein siRNA 5′ RNA antisense
uuaauaggcgugugcuuagaa

SEQ ID NO: 851 - RNA, 21 nt, Artificial Sequence
  Spike (S) protein siRNA 5′ RNA antisense
uucaacguacacuuuguuucu

SEQ ID NO: 852 - RNA, 21 nt, Artificial Sequence
  Spike (S) protein siRNA 5′ RNA antisense
uuucaacguacacuuuguuuc

SEQ ID NO: 853 - RNA, 21 nt, Artificial Sequence
  Spike (S) protein siRNA 5′ RNA antisense
uucuacagugaaggauuucaa

SEQ ID NO: 854 - RNA, 21 nt, Artificial Sequence
  Spike (S) protein siRNA 5′ RNA antisense
uuucuacagugaaggauuuca

SEQ ID NO: 855 - RNA, 21 nt, Artificial Sequence
  Spike (S) protein siRNA 5′ RNA antisense
uugcugauucucuuccuguuc

SEQ ID NO: 856 - RNA, 21 nt, Artificial Sequence
  Spike (S) protein siRNA 5′ RNA antisense
uuaguaggagacacuccauaa

SEQ ID NO: 857 - RNA, 21 nt, Artificial Sequence
  Spike (S) protein siRNA 5′ RNA antisense
uuuaguaggagacacuccaua

SEQ ID NO: 858 - RNA, 21 nt, Artificial Sequence
  Spike (S) protein siRNA 5′ RNA antisense
uuugucugacuucaucaccuc

SEQ ID NO: 859 - RNA, 21 nt, Artificial Sequence
  Spike (S) protein siRNA 5′ RNA antisense
uuacaaggugugcuaccggcc

SEQ ID NO: 860 - RNA, 21 nt, Artificial Sequence
  Spike (S) protein siRNA 5′ RNA antisense
uuaguagacuuuuuaggucca

SEQ ID NO: 861 - RNA, 21 nt, Artificial Sequence
  Spike (S) protein siRNA 5′ RNA antisense
uugacacauuuguuuuuaacc

SEQ ID NO: 862 - RNA, 21 nt, Artificial Sequence
  Spike (S) protein siRNA 5′ RNA antisense
uuagacucaguaagaacaccu

SEQ ID NO: 863 - RNA, 21 nt, Artificial Sequence
  Spike (S) protein siRNA 5′ RNA antisense
uuguuagacucaguaagaaca

SEQ ID NO: 864 - RNA, 21 nt, Artificial Sequence
  Spike (S) protein siRNA 5′ RNA antisense
uuuguuccugguguuauaaca

SEQ ID NO: 865 - RNA, 21 nt, Artificial Sequence
  Spike (S) protein siRNA 5′ RNA antisense
uuagucugagucugauaacua

SEQ ID NO: 866 - RNA, 21 nt, Artificial Sequence
  Spike (S) protein siRNA 5′ RNA antisense
uuuucugcaccaagugacaua

SEQ ID NO: 867 - RNA, 21 nt, Artificial Sequence
  Spike (S) protein siRNA 5′ RNA antisense
uuggucauagacacugguaga

SEQ ID NO: 868 - RNA, 21 nt, Artificial Sequence
  Spike (S) protein siRNA 5′ RNA antisense
uuguucaacagcuauuccagu

SEQ ID NO: 869 - RNA, 21 nt, Artificial Sequence
  Spike (S) protein siRNA 5′ RNA antisense
uugucuuguucaacagcuauu

SEQ ID NO: 870 - RNA, 21 nt, Artificial Sequence
  Spike (S) protein siRNA 5′ RNA antisense
uuugucuuguucaacagcuau

SEQ ID NO: 871 - RNA, 21 nt, Artificial Sequence
  Spike (S) protein siRNA 5′ RNA antisense
uugauggaucugguaauauuu

SEQ ID NO: 872 - RNA, 21 nt, Artificial Sequence
  Spike (S) protein siRNA 5′ RNA antisense
uuugauggaucugguaauauu

SEQ ID NO: 873 - RNA, 21 nt, Artificial Sequence
  Spike (S) protein siRNA 5′ RNA antisense
uuuugauggaucugguaauau

SEQ ID NO: 874 - RNA, 21 nt, Artificial Sequence
  Spike (S) protein siRNA 5′ RNA antisense
uugguuuugauggaucuggua

SEQ ID NO: 875 - RNA, 21 nt, Artificial Sequence
  Spike (S) protein siRNA 5′ RNA antisense
uugaugaagccagcaucugca

SEQ ID NO: 876 - RNA, 21 nt, Artificial Sequence
  Spike (S) protein siRNA 5′ RNA antisense
uugagcaaucauuucaucugu

SEQ ID NO: 877 - RNA, 21 nt, Artificial Sequence
  Spike (S) protein siRNA 5′ RNA antisense
uuguaaugcagcaccugcacc

SEQ ID NO: 878 - RNA, 21 nt, Artificial Sequence
  Spike (S) protein siRNA 5′ RNA antisense
uuuugccaauagcacuauuaa

SEQ ID NO: 879 - RNA, 21 nt, Artificial Sequence
  Spike (S) protein siRNA 5′ RNA antisense
uuuccaagugcacuugcugug

SEQ ID NO: 880 - RNA, 21 nt, Artificial Sequence
  Spike (S) protein siRNA 5′ RNA antisense
uuuuccaagugcacuugcugu

SEQ ID NO: 881 - RNA, 21 nt, Artificial Sequence
  Spike (S) protein siRNA 5′ RNA antisense
uuugguugaccacaucuugaa

SEQ ID NO: 882 - RNA, 21 nt, Artificial Sequence
  Spike (S) protein siRNA 5′ RNA antisense
uuuugguugaccacaucuuga

SEQ ID NO: 883 - RNA, 21 nt, Artificial Sequence
  Spike (S) protein siRNA 5′ RNA antisense
uugugcauuuugguugaccac

SEQ ID NO: 884 - RNA, 21 nt, Artificial Sequence
  Spike (S) protein siRNA 5′ RNA antisense
uuugcacuucagccucaacuu

SEQ ID NO: 885 - RNA, 21 nt, Artificial Sequence
  Spike (S) protein siRNA 5′ RNA antisense
uugaagucugccugugaucaa

SEQ ID NO: 886 - RNA, 21 nt, Artificial Sequence
  Spike (S) protein siRNA 5′ RNA antisense
uuugaagucugccugugauca

SEQ ID NO: 887 - RNA, 21 nt, Artificial Sequence
  Spike (S) protein siRNA 5′ RNA antisense
uuguugagucacauaugucug

SEQ ID NO: 888 - RNA, 21 nt, Artificial Sequence
  Spike (S) protein siRNA 5′ RNA antisense
uuagcagaagcucugauuucu

SEQ ID NO: 889 - RNA, 21 nt, Artificial Sequence
  Spike (S) protein siRNA 5′ RNA antisense
uuaguagcagcaagauuagca

SEQ ID NO: 890 - RNA, 21 nt, Artificial Sequence
  Spike (S) protein siRNA 5′ RNA antisense
uuuaguagcagcaagauuagc

SEQ ID NO: 891 - RNA, 21 nt, Artificial Sequence
  Spike (S) protein siRNA 5′ RNA antisense
uugauuguccaaguacacacu

SEQ ID NO: 892 - RNA, 21 nt, Artificial Sequence
  Spike (S) protein siRNA 5′ RNA antisense
uugugaaguucuuuucuugug

SEQ ID NO: 893 - RNA, 21 nt, Artificial Sequence
  Spike (S) protein siRNA 5′ RNA antisense
uugucuguaguaaugauuugu

SEQ ID NO: 894 - RNA, 21 nt, Artificial Sequence
  Spike (S) protein siRNA 5′ RNA antisense
uuguugacaauuccuauuaca

SEQ ID NO: 895 - RNA, 21 nt, Artificial Sequence
  Spike (S) protein siRNA 5′ RNA antisense
uugaaugagucuaauucaggu

SEQ ID NO: 896 - RNA, 21 nt, Artificial Sequence
  Spike (S) protein siRNA 5′ RNA antisense
uuaaugccagagaugucaccu

SEQ ID NO: 897 - RNA, 21 nt, Artificial Sequence
  Spike (S) protein siRNA 5′ RNA antisense
uuacaacugaagcauuaaugc

SEQ ID NO: 898 - RNA, 21 nt, Artificial Sequence
  Spike (S) protein siRNA 5′ RNA antisense
uuccaaguucuuggagaucga

SEQ ID NO: 899 - RNA, 21 nt, Artificial Sequence
  Spike (S) protein siRNA 5′ RNA antisense
uuuccaaguucuuggagaucg

SEQ ID NO: 900 - RNA, 21 nt, Artificial Sequence
  Spike (S) protein siRNA 5′ RNA antisense
uugagacaacuacagcaacug

SEQ ID NO: 901 - RNA, 19 nt, Artificial Sequence
  Spike (S) protein siRNA 5′ RNA antisense
uuacaaggugugcuaccgg

SEQ ID NO: 902 - RNA, 22 nt, Artificial Sequence
  Spike (S) protein siRNA 5′ RNA antisense
uuacaaggugugcuaccggccu

SEQ ID NO: 903 - RNA, 25 nt, Artificial Sequence
  Spike (S) protein siRNA 5′ RNA antisense
uuacaaggugugcuaccggccugau

SEQ ID NO: 904 - RNA, 19 nt, Artificial Sequence
  Spike (S) protein siRNA 5′ RNA antisense
uugaaugagucuaauucag

SEQ ID NO: 905 - RNA, 22 nt, Artificial Sequence
  Spike (S) protein siRNA 5′ RNA antisense
uugaaugagucuaauucagguu

SEQ ID NO: 906 - RNA, 25 nt, Artificial Sequence
  Spike (S) protein siRNA 5′ RNA antisense
uugaaugagucuaauucagguugca

SEQ ID NO: 907 - RNA, 25 nt, Artificial Sequence
  Spike (S) protein siRNA 5′ RNA antisense
uccuugaaugagucuaauucagguu

SEQ ID NO: 908 - RNA, 20 nt, Artificial Sequence
  Spike (S) protein siRNA 5′ RNA antisense
cucauugaggcggucaauuu

SEQ ID NO: 909 - RNA, 19 nt, Artificial Sequence
  Spike (S) protein siRNA 5′ RNA antisense
cucauugaggcggucaauu

SEQ ID NO: 910 - RNA, 21 nt, Artificial Sequence
  Spike (S) protein siRNA 5′ RNA antisense
agcauggaaccaaguaacauu

SEQ ID NO: 911 - RNA, 21 nt, Artificial Sequence
  Spike (S) protein siRNA 5′ RNA antisense
ccauccaacuuuuguuguuuu

SEQ ID NO: 912 - RNA, 21 nt, Artificial Sequence
  Spike (S) protein siRNA 5′ RNA antisense
uccauccaacuuuuguuguuu

SEQ ID NO: 913 - RNA, 21 nt, Artificial Sequence
  Spike (S) protein siRNA 5′ RNA antisense
uuccauccaacuuuuguuguu

SEQ ID NO: 914 - RNA, 21 nt, Artificial Sequence
  Spike (S) protein siRNA 5′ RNA antisense
cgugugcuuagaauauauuuu

SEQ ID NO: 915 - RNA, 21 nt, Artificial Sequence
  Spike (S) protein siRNA 5′ RNA antisense
ugguuccauuuucauuauauu

SEQ ID NO: 916 - RNA, 21 nt, Artificial Sequence
  Spike (S) protein siRNA 5′ RNA antisense
agcaucuguaaugguuccauu

SEQ ID NO: 917 - RNA, 21 nt, Artificial Sequence
  Spike (S) protein siRNA 5′ RNA antisense
ucuacagcaucuguaaugguu

SEQ ID NO: 918 - RNA, 21 nt, Artificial Sequence
  Spike (S) protein siRNA 5′ RNA antisense
ucagcaacacaguugcugauu

SEQ ID NO: 919 - RNA, 21 nt, Artificial Sequence
  Spike (S) protein siRNA 5′ RNA antisense
aucaucugguaauuuauaauu

SEQ ID NO: 920 - RNA, 21 nt, Artificial Sequence
  Spike (S) protein siRNA 5′ RNA antisense
accaaccuuagaaucaagauu

SEQ ID NO: 921 - RNA, 21 nt, Artificial Sequence
  Spike (S) protein siRNA 5′ RNA antisense
ugguugguaaccaacaccauu

SEQ ID NO: 922 - RNA, 21 nt, Artificial Sequence
  Spike (S) protein siRNA 5′ RNA antisense
accugguuagaaguauuuguu

SEQ ID NO: 923 - RNA, 21 nt, Artificial Sequence
  Spike (S) protein siRNA 5′ RNA antisense
gucacacucauaugaguuguu

SEQ ID NO: 924 - RNA, 21 nt, Artificial Sequence
  Spike (S) protein siRNA 5′ RNA antisense
uaguguaggcaaugauggauu

SEQ ID NO: 925 - RNA, 21 nt, Artificial Sequence
  Spike (S) protein siRNA 5′ RNA antisense
aaugaccucuugcuugguuuu

SEQ ID NO: 926 - RNA, 21 nt, Artificial Sequence
  Spike (S) protein siRNA 5′ RNA antisense
aucugcaagugucacuuuguu

SEQ ID NO: 927 - RNA, 21 nt, Artificial Sequence
  Spike (S) protein siRNA 5′ RNA antisense
cagcaucugcaagugucacuu

SEQ ID NO: 928 - RNA, 21 nt, Artificial Sequence
  Spike (S) protein siRNA 5′ RNA antisense
aaggcaaucaccauauuguuu

SEQ ID NO: 929 - RNA, 21 nt, Artificial Sequence
  Spike (S) protein siRNA 5′ RNA antisense
aagguccaaccagaagugauu

SEQ ID NO: 930 - RNA, 21 nt, Artificial Sequence
  Spike (S) protein siRNA 5′ RNA antisense
uugguucucauagagaacauu

SEQ ID NO: 931 - RNA, 21 nt, Artificial Sequence
  Spike (S) protein siRNA 5′ RNA antisense
uugaccacaucuugaaguuuu

SEQ ID NO: 932 - RNA, 21 nt, Artificial Sequence
  Spike (S) protein siRNA 5′ RNA antisense
guugaccacaucuugaaguuu

SEQ ID NO: 933 - RNA, 21 nt, Artificial Sequence
  Spike (S) protein siRNA 5′ RNA antisense
gguugaccacaucuugaaguu

SEQ ID NO: 934 - RNA, 21 nt, Artificial Sequence
  Spike (S) protein siRNA 5′ RNA antisense
uugcacuucagccucaacuuu

SEQ ID NO: 935 - RNA, 21 nt, Artificial Sequence
  Spike (S) protein siRNA 5′ RNA antisense
ccugugaucaaccuaucaauu

SEQ ID NO: 936 - RNA, 21 nt, Artificial Sequence
  Spike (S) protein siRNA 5′ RNA antisense
uuucugcagcucuaauuaauu

SEQ ID NO: 937 - RNA, 21 nt, Artificial Sequence
  Spike (S) protein siRNA 5′ RNA antisense
gagcaguugugaaguucuuuu

SEQ ID NO: 938 - RNA, 21 nt, Artificial Sequence
  Spike (S) protein siRNA 5′ RNA antisense
ggagcaguugugaaguucuuu

SEQ ID NO: 939 - RNA, 21 nt, Artificial Sequence
  Spike (S) protein siRNA 5′ RNA antisense
uguugucuguaguaaugauuu

SEQ ID NO: 940 - RNA, 21 nt, Artificial Sequence
  Spike (S) protein siRNA 5′ RNA antisense
ccuccuugaaugagucuaauu

SEQ ID NO: 941 - RNA, 21 nt, Artificial Sequence
  Spike (S) protein siRNA 5′ RNA antisense
aucuggugauguaugauucuu

SEQ ID NO: 942 - RNA, 21 nt, Artificial Sequence
  Spike (S) protein siRNA 5′ RNA antisense
aacaucuggugauguaugauu

SEQ ID NO: 943 - RNA, 21 nt, Artificial Sequence
  Spike (S) protein siRNA 5′ RNA antisense
cauugaggcggucaauuucuu

SEQ ID NO: 944 - RNA, 21 nt, Artificial Sequence
  Spike (S) protein siRNA 5′ RNA antisense
accucauugaggcggucaauu

SEQ ID NO: 945 - RNA, 21 nt, Artificial Sequence
  Spike (S) protein siRNA 5′ RNA antisense
agaucgaugagagauucauuu

SEQ ID NO: 946 - RNA, 21 nt, Artificial Sequence
  Spike (S) protein siRNA 5′ RNA antisense
acugcucauacuuuccaaguu

SEQ ID NO: 947 - RNA, 21 nt, Artificial Sequence
  Spike (S) protein siRNA 5′ RNA antisense
uucugguuguaagauuaacac

SEQ ID NO: 948 - RNA, 21 nt, Artificial Sequence
  Spike (S) protein siRNA 5′ RNA antisense
uuaguagcguuauuaacaaua

SEQ ID NO: 949 - RNA, 21 nt, Artificial Sequence
  Spike (S) protein siRNA 5′ RNA antisense
uucacagacuuuaauaacaac

SEQ ID NO: 950 - RNA, 21 nt, Artificial Sequence
  Spike (S) protein siRNA 5′ RNA antisense
uuccauccaacuuuuguuguu

SEQ ID NO: 951 - RNA, 21 nt, Artificial Sequence
  Spike (S) protein siRNA 5′ RNA antisense
uuuccauccaacuuuuguugu

SEQ ID NO: 952 - RNA, 21 nt, Artificial Sequence
  Spike (S) protein siRNA 5′ RNA antisense
uucucuuccuguuccaagcau

SEQ ID NO: 953 - RNA, 21 nt, Artificial Sequence
  Spike (S) protein siRNA 5′ RNA antisense
uugucugacuucaucaccucu

SEQ ID NO: 954 - RNA, 21 nt, Artificial Sequence
  Spike (S) protein siRNA 5′ RNA antisense
uuccaagcuauaacgcagccu

SEQ ID NO: 955 - RNA, 21 nt, Artificial Sequence
  Spike (S) protein siRNA 5′ RNA antisense
uuaccaccaaccuuagaauca

SEQ ID NO: 956 - RNA, 21 nt, Artificial Sequence
  Spike (S) protein siRNA 5′ RNA antisense
uucaacaccauuacaaggugu

SEQ ID NO: 957 - RNA, 21 nt, Artificial Sequence
  Spike (S) protein siRNA 5′ RNA antisense
uugguaaccaacaccauuagu

SEQ ID NO: 958 - RNA, 21 nt, Artificial Sequence
  Spike (S) protein siRNA 5′ RNA antisense
uuguuccugguguuauaacac

SEQ ID NO: 959 - RNA, 21 nt, Artificial Sequence
  Spike (S) protein siRNA 5′ RNA antisense
uucugugcaguuaacauccug

SEQ ID NO: 960 - RNA, 21 nt, Artificial Sequence
  Spike (S) protein siRNA 5′ RNA antisense
uugaucugcaugaauagcaac

SEQ ID NO: 961 - RNA, 21 nt, Artificial Sequence
  Spike (S) protein siRNA 5′ RNA antisense
uugacuagcuacacuacgugc

SEQ ID NO: 962 - RNA, 21 nt, Artificial Sequence
  Spike (S) protein siRNA 5′ RNA antisense
uuucugcaccaagugacauag

SEQ ID NO: 963 - RNA, 21 nt, Artificial Sequence
  Spike (S) protein siRNA 5′ RNA antisense
uucugugguaacacuaauagu

SEQ ID NO: 964 - RNA, 21 nt, Artificial Sequence
  Spike (S) protein siRNA 5′ RNA antisense
uuucugugguaacacuaauag

SEQ ID NO: 965 - RNA, 21 nt, Artificial Sequence
  Spike (S) protein siRNA 5′ RNA antisense
uugcugcauucaguugaauca

SEQ ID NO: 966 - RNA, 21 nt, Artificial Sequence
  Spike (S) protein siRNA 5′ RNA antisense
uucaacagcuauuccaguuaa

SEQ ID NO: 967 - RNA, 21 nt, Artificial Sequence
  Spike (S) protein siRNA 5′ RNA antisense
uugcuugguuuugauggaucu

SEQ ID NO: 968 - RNA, 21 nt, Artificial Sequence
  Spike (S) protein siRNA 5′ RNA antisense
uugguucucauagagaacauu

SEQ ID NO: 969 - RNA, 21 nt, Artificial Sequence
  Spike (S) protein siRNA 5′ RNA antisense
uuccaagugcacuugcugugg

SEQ ID NO: 970 - RNA, 21 nt, Artificial Sequence
  Spike (S) protein siRNA 5′ RNA antisense
uugaccacaucuugaaguuuu

SEQ ID NO: 971 - RNA, 21 nt, Artificial Sequence
  Spike (S) protein siRNA 5′ RNA antisense
uugguugaccacaucuugaag

SEQ ID NO: 972 - RNA, 21 nt, Artificial Sequence
  Spike (S) protein siRNA 5′ RNA antisense
uuggagcuaaguuguuuaaca

SEQ ID NO: 973 - RNA, 21 nt, Artificial Sequence
  Spike (S) protein siRNA 5′ RNA antisense
uugcacuucagccucaacuuu

SEQ ID NO: 974 - RNA, 21 nt, Artificial Sequence
  Spike (S) protein siRNA 5′ RNA antisense
uugagucacauaugucugcaa

SEQ ID NO: 975 - RNA, 21 nt, Artificial Sequence
  Spike (S) protein siRNA 5′ RNA antisense
uuucugcagcucuaauuaauu

SEQ ID NO: 976 - RNA, 21 nt, Artificial Sequence
  Spike (S) protein siRNA 5′ RNA antisense
uuguccaaguacacacucuga

SEQ ID NO: 977 - RNA, 21 nt, Artificial Sequence
  Spike (S) protein siRNA 5′ RNA antisense
uuggagaucgaugagagauuc

SEQ ID NO: 978 - RNA, 21 nt, Artificial Sequence
  Spike (S) protein siRNA 5′ RNA antisense
uucuuggagaucgaugagaga

SEQ ID NO: 979 - RNA, 21 nt, Artificial Sequence
  Spike (S) protein siRNA 5′ RNA antisense
uuugcagcaggauccacaaga

SEQ ID NO: 980 - RNA, 21 nt, Artificial Sequence
  Spike (S) protein siRNA 5′ RNA antisense
uuugacuccuuugagcacugg

SEQ ID NO: 981 - RNA, 19 nt, Artificial Sequence
  Spike (S) protein siRNA 5′ RNA antisense
uaaguagggacugggucuu

SEQ ID NO: 982 - RNA, 19 nt, Artificial Sequence
  Spike (S) protein siRNA 5′ RNA antisense
uuaguagcguuauuaacaa

SEQ ID NO: 983 - RNA, 21 nt, Artificial Sequence
  Spike (S) protein siRNA 5′ RNA antisense
uuaguagcguuauuaacaaua

SEQ ID NO: 984 - RNA, 25 nt, Artificial Sequence
  Spike (S) protein siRNA 5′ RNA antisense
uuaguagcguuauuaacaauaagua

SEQ ID NO: 985 - RNA, 19 nt, Artificial Sequence
  Spike (S) protein siRNA 5′ RNA antisense
uucaacaccauuacaaggu

SEQ ID NO: 986 - RNA, 19 nt, Artificial Sequence
  Spike (S) protein siRNA 5′ RNA antisense
ggccugauagauuucaguu

SEQ ID NO: 987 - RNA, 21 nt, Artificial Sequence
  Spike (S) protein siRNA 5′ RNA antisense
uucaacaccauuacaaggugu

SEQ ID NO: 988 - RNA, 25 nt, Artificial Sequence
  Spike (S) protein siRNA 5′ RNA antisense
uucaacaccauuacaaggugugcua

SEQ ID NO: 989 - RNA, 25 nt, Artificial Sequence
  Spike (S) protein siRNA 5′ RNA antisense
ggugugcuaccggccugauagauuu

SEQ ID NO: 990 - RNA, 25 nt, Artificial Sequence
  Spike (S) protein siRNA 5′ RNA antisense
gcaaccucauugaggcggucaauuu

SEQ ID NO: 991 - RNA, 21 nt, Artificial Sequence
  Spike (S) protein siRNA 5′ RNA antisense
gcgugugcuuagaauauauuu

SEQ ID NO: 992 - RNA, 21 nt, Artificial Sequence
  Spike (S) protein siRNA 5′ RNA antisense
aggagacacuccauaacacuu

SEQ ID NO: 993 - RNA, 21 nt, Artificial Sequence
  Spike (S) protein siRNA 5′ RNA antisense
cacgccaaguaggaguaaguu

SEQ ID NO: 994 - RNA, 21 nt, Artificial Sequence
  Spike (S) protein siRNA 5′ RNA antisense
agcaucugcaagugucacuuu

SEQ ID NO: 995 - RNA, 21 nt, Artificial Sequence
  Spike (S) protein siRNA 5′ RNA antisense
caccaaggcaaucaccauauu

SEQ ID NO: 996 - RNA, 21 nt, Artificial Sequence
  Spike (S) protein siRNA 5′ RNA antisense
cugcagcucuaauuaauuguu

SEQ ID NO: 997 - RNA, 21 nt, Artificial Sequence
  Spike (S) protein siRNA 5′ RNA antisense
aggagcaguugugaaguucuu

SEQ ID NO: 998 - RNA, 21 nt, Artificial Sequence
  Spike (S) protein siRNA 5′ RNA antisense
auugaggcggucaauuucuuu

SEQ ID NO: 999 - RNA, 21 nt, Artificial Sequence
  Spike (S) protein siRNA 5′ RNA antisense
uugcuggugcauguagaaguu

SEQ ID NO: 1000 - RNA, 21 nt, Artificial Sequence
  Spike (S) protein siRNA 5′ RNA antisense
uuccugguguuauaacacuga

SEQ ID NO: 1001 - RNA, 21 nt, Artificial Sequence
  Spike (S) protein siRNA 5′ RNA antisense
uucugcaccaagugacauagu

SEQ ID NO: 1002 - RNA, 21 nt, Artificial Sequence
  Spike (S) protein siRNA 5′ RNA antisense
uucagccucaacuuugucaag

SEQ ID NO: 1003 - RNA, 21 nt, Artificial Sequence
  Spike (S) protein siRNA 5′ RNA antisense
uucugcagcucuaauuaauug

SEQ ID NO: 1004 - RNA, 21 nt, Artificial Sequence
  Spike (S) protein siRNA 5′ RNA antisense
uugcagcaggauccacaagaa

SEQ ID NO: 1005 - RNA, 21 nt, Artificial Sequence
  Spike (S) protein siRNA 5′ RNA antisense
gugcuaccggccugauagauu

SEQ ID NO: 1006 - RNA, 22 nt, Artificial Sequence
  Spike (S) protein siRNA 5′ RNA antisense
gugcuaccggccugauagauuu

SEQ ID NO: 1007 - RNA, 22 nt, Artificial Sequence
  Spike (S) protein siRNA 5′ RNA antisense
ugugcuaccggccugauagauu

SEQ ID NO: 1008 - RNA, 25 nt, Artificial Sequence
  Spike (S) protein siRNA 5′ RNA antisense
aggugugcuaccggccugauagauu

SEQ ID NO: 1009 - RNA, 25 nt, Artificial Sequence
  Spike (S) protein siRNA 5′ RNA antisense
ggcaaccucauugaggcggucaauu

SEQ ID NO: 1010 - RNA, 21 nt, Artificial Sequence
  Spike (S) protein siRNA 5′ RNA antisense
ccggccugauagauuucaguu

SEQ ID NO: 1011 - RNA, 21 nt, Artificial Sequence
  Spike (S) protein siRNA 5′ RNA antisense
ugcuaccggccugauagauuu

SEQ ID NO: 1012 - RNA, 21 nt, Artificial Sequence
  Spike (S) protein siRNA 5′ RNA antisense
ggcaggagcaguugugaaguu

SEQ ID NO: 1013 - RNA, 21 nt, Artificial Sequence
  Spike (S) protein siRNA 5′ RNA antisense
uugaggcggucaauuucuuuu

SEQ ID NO: 1014 - RNA, 21 nt, Artificial Sequence
  Spike (S) protein siRNA 5′ RNA antisense
uugaggcggucaauuucuuuu

SEQ ID NO: 1015 - RNA, 19 nt, Artificial Sequence
  Spike (S) protein siRNA 5′ RNA antisense
cuaccggccugauagauuu

SEQ ID NO: 1016 - RNA, 19 nt, Artificial Sequence
  Spike (S) protein siRNA 5′ RNA antisense
gcuaccggccugauagauu

SEQ ID NO: 1017 - RNA, 25 nt, Artificial Sequence
  Spike (S) protein siRNA 5′ RNA antisense
gcuaccggccugauagauuucaguu